SEQUENCE LISTING (497 sequences)

SEQ ID NO: 1 - protein, 25 aa, Homo sapiens
  MOD_RES (18)..(18): Phosphorylated-Tyr
FMQTFVLAPEGSVANKFYVHNDIFR

SEQ ID NO: 2 - protein, 28 aa, Homo sapiens
  MOD_RES (20)..(20): Phosphorylated-Tyr
NSSYVHGGVDASGKPQEAVYGQNDIHHK

SEQ ID NO: 3 - protein, 17 aa, Homo sapiens
  MOD_RES (10)..(10): Phosphorylated-Tyr
MSGDPDVLEYYKNDHSK

SEQ ID NO: 4 - protein, 21 aa, Homo sapiens
  MOD_RES (11)..(11): Phosphorylated-Tyr
SESELLFLPDYLVLSNCETGR

SEQ ID NO: 5 - protein, 21 aa, Homo sapiens
  MOD_RES (14)..(14): Phosphorylated-Tyr
FFANPVSREDEESYIEMEEHR

SEQ ID NO: 6 - protein, 21 aa, Homo sapiens
  MOD_RES (3)..(3): Phosphorylated-Tyr
LEYEEHKKEYEDAENTSTQSK

SEQ ID NO: 7 - protein, 21 aa, Homo sapiens
  MOD_RES (10)..(10): Phosphorylated-Tyr
LEYEEHKKEYEDAENTSTQSK

SEQ ID NO: 8 - protein, 11 aa, Homo sapiens
  MOD_RES (1)..(1): Phosphorylated-Tyr
YLQHHHFHQER

SEQ ID NO: 9 - protein, 20 aa, Homo sapiens
  MOD_RES (13)..(13): Phosphorylated-Tyr
LVHGLVVKDPSIYDTLESVR

SEQ ID NO: 10 - protein, 10 aa, Homo sapiens
  MOD_RES (6)..(6): Phosphorylated-Tyr
YGMLLYQNYR

SEQ ID NO: 11 - protein, 10 aa, Homo sapiens
  MOD_RES (4)..(4): Phosphorylated-Tyr
YDSYESCDSR

SEQ ID NO: 12 - protein, 25 aa, Homo sapiens
  MOD_RES (4)..(4): Phosphorylated-Tyr
RGGYSTLPNPQKSEWIYDTPVSPGK

SEQ ID NO: 13 - protein, 30 aa, Homo sapiens
  MOD_RES (24)..(24): Phosphorylated-Tyr
NTPLTSFAEESRPHALPSSSSTFYNPPSGR

SEQ ID NO: 14 - protein, 16 aa, Homo sapiens
  MOD_RES (11)..(11): Phosphorylated-Tyr
ESPVSAGNDAYVDLDR

SEQ ID NO: 15 - protein, 22 aa, Homo sapiens
  MOD_RES (20)..(20): Phosphorylated-Tyr
SAVGFDYKGEVEKHTSQKDYSR

SEQ ID NO: 16 - protein, 18 aa, Homo sapiens
  MOD_RES (5)..(5): Phosphorylated-Tyr
AALGYDYKGETEKHESQR

SEQ ID NO: 17 - protein, 22 aa, Homo sapiens
  MOD_RES (7)..(7): Phosphorylated-Tyr
AALGYDYKGETEKHESQRDYAK

SEQ ID NO: 18 - protein, 22 aa, Homo sapiens
  MOD_RES (20)..(20): Phosphorylated-Tyr
AALGYDYKGETEKHESQRDYAK

SEQ ID NO: 19 - protein, 16 aa, Homo sapiens
  MOD_RES (12)..(12): Phosphorylated-Tyr
GPSTLTAPNGHYILSR

SEQ ID NO: 20 - protein, 41 aa, Homo sapiens
  MOD_RES (39)..(39): Phosphorylated-Tyr
VDPNGYMMMSPSGGCSPDIGGGPSSSSSSSNAVPSGTSYGK

SEQ ID NO: 21 - protein, 21 aa, Homo sapiens
  MOD_RES (3)..(3): Phosphorylated-Tyr
APYTCGGDSDQYVLMSSPVGR

SEQ ID NO: 22 - protein, 15 aa, Homo sapiens
  MOD_RES (6)..(6): Phosphorylated-Tyr
SPLSDYMNLDFSSPK

SEQ ID NO: 23 - protein, 12 aa, Homo sapiens
  MOD_RES (5)..(5): Phosphorylated-Tyr
SMSGYLSGFQAR

SEQ ID NO: 24 - protein, 18 aa, Homo sapiens
  MOD_RES (11)..(11): Phosphorylated-Tyr
QFDNLKPSGGYITGDQAR

SEQ ID NO: 25 - protein, 15 aa, Homo sapiens
  MOD_RES (14)..(14): Phosphorylated-Tyr
ETYNTQQLALEQLYK

SEQ ID NO: 26 - protein, 25 aa, Homo sapiens
  MOD_RES (20)..(20): Phosphorylated-Tyr
ETQTHENMSQLSEEEQNKDYQDCSK

SEQ ID NO: 27 - protein, 16 aa, Homo sapiens
  MOD_RES (14)..(14): Phosphorylated-Tyr
LPGSYDSTSSDSLYPR

SEQ ID NO: 28 - protein, 15 aa, Homo sapiens
  MOD_RES (3)..(3): Phosphorylated-Tyr
NIYDILPWRQEDLGR

SEQ ID NO: 29 - protein, 27 aa, Homo sapiens
  MOD_RES (19)..(19): Phosphorylated-Tyr
GGMDYAYIPPPGLQPEPGYGYAPNQGR

SEQ ID NO: 30 - protein, 27 aa, Homo sapiens
  MOD_RES (21)..(21): Phosphorylated-Tyr
GGMDYAYIPPPGLQPEPGYGYAPNQGR

SEQ ID NO: 31 - protein, 33 aa, Homo sapiens
  MOD_RES (1)..(1): Phosphorylated-Tyr
YYEGYYAAGPGYGGRNDSDPTYGQQGHPNTWKR

SEQ ID NO: 32 - protein, 25 aa, Homo sapiens
  MOD_RES (5)..(5): Phosphorylated-Tyr
REPGYTPPGAGNQNPPGMYPVTGPK

SEQ ID NO: 33 - protein, 13 aa, Homo sapiens
  MOD_RES (1)..(1): Phosphorylated-Tyr
YADITVTSSKALR

SEQ ID NO: 34 - protein, 14 aa, Homo sapiens
  MOD_RES (8)..(8): Phosphorylated-Tyr
TAEAQLAYELQGAR

SEQ ID NO: 35 - protein, 9 aa, Homo sapiens
  MOD_RES (1)..(1): Phosphorylated-Tyr
YNVRYELAR

SEQ ID NO: 36 - protein, 9 aa, Homo sapiens
  MOD_RES (5)..(5): Phosphorylated-Tyr
YNVRYELAR

SEQ ID NO: 37 - protein, 15 aa, Homo sapiens
  MOD_RES (2)..(2): Phosphorylated-Tyr
WYQFRVAAVNVHGTR

SEQ ID NO: 38 - protein, 14 aa, Homo sapiens
  MOD_RES (6)..(6): Phosphorylated-Tyr
CKPGFYNLKEKNPR

SEQ ID NO: 39 - protein, 31 aa, Homo sapiens
  MOD_RES (7)..(7): Phosphorylated-Tyr
AEAAAPYTVLAQSAPREDGYSDASGFGYCFR

SEQ ID NO: 40 - protein, 31 aa, Homo sapiens; identical to SEQ ID NO: 39
  MOD_RES (20)..(20): Phosphorylated-Tyr

SEQ ID NO: 41 - protein, 31 aa, Homo sapiens; identical to SEQ ID NO: 39
  MOD_RES (28)..(28): Phosphorylated-Tyr

SEQ ID NO: 42 - protein, 21 aa, Homo sapiens
  MOD_RES (13)..(13): Phosphorylated-Tyr
LTESPCALVASQYGWSGNMER

SEQ ID NO: 43 - protein, 19 aa, Homo sapiens
  MOD_RES (4)..(4): Phosphorylated-Tyr
AQAYQTGKDISTNYYASQK

SEQ ID NO: 44 - protein, 19 aa, Homo sapiens
  MOD_RES (14)..(14): Phosphorylated-Tyr
AQAYQTGKDISTNYYASQK

SEQ ID NO: 45 - protein, 14 aa, Homo sapiens
  MOD_RES (6)..(6): Phosphorylated-Tyr
HGDEIYIAPSGVQK

SEQ ID NO: 46 - protein, 9 aa, Homo sapiens
  MOD_RES (7)..(7): Phosphorylated-Tyr
IKEQEDYIR

SEQ ID NO: 47 - protein, 19 aa, Homo sapiens
  MOD_RES (5)..(5): Phosphorylated-Tyr
GTPLYGQPSWWGDDEVDEK

SEQ ID NO: 48 - protein, 23 aa, Homo sapiens
  MOD_RES (15)..(15): Phosphorylated-Tyr
KMIDKVFGVDDNQDYNRPVINEK

SEQ ID NO: 49 - protein, 13 aa, Homo sapiens
  MOD_RES (9)..(9): Phosphorylated-Tyr
AVGASFPLYEPAK

SEQ ID NO: 50 - protein, 13 aa, Homo sapiens
  MOD_RES (7)..(7): Phosphorylated-Tyr
VNSMVAYKIPVND

SEQ ID NO: 51 - protein, 13 aa, Homo sapiens
  MOD_RES (7)..(7): Phosphorylated-Tyr
VSGVDGYETEGIR

SEQ ID NO: 52 - protein, 25 aa, Homo sapiens
  MOD_RES (14)..(14): Phosphorylated-Tyr
GSTEGSESYEEDPYLVVNPNYLLED

SEQ ID NO: 53 - protein, 11 aa, Homo sapiens
  MOD_RES (10)..(10): Phosphorylated-Tyr
CVLNEGMPIYR

SEQ ID NO: 54 - protein, 10 aa, Homo sapiens
  MOD_RES (5)..(5): Phosphorylated-Tyr
VQVEYKGETK

SEQ ID NO: 55 - protein, 16 aa, Homo sapiens
  MOD_RES (8)..(8): Phosphorylated-Tyr
MVQEAEKYKAEDEKQR

SEQ ID NO: 56 - protein, 20 aa, Homo sapiens
  MOD_RES (8)..(8): Phosphorylated-Tyr
NAVEEYVYEFRDKLCGPYEK

SEQ ID NO: 57 - protein, 18 aa, Homo sapiens
  MOD_RES (10)..(10): Phosphorylated-Tyr
LLTETEDWLYEEGEDQAK

SEQ ID NO: 58 - protein, 11 aa, Homo sapiens
  MOD_RES (5)..(5): Phosphorylated-Tyr
ENLSYDLVPLK

SEQ ID NO: 59 - protein, 22 aa, Homo sapiens
  MOD_RES (12)..(12): Phosphorylated-Tyr
AAAIGIDLGTTYSCVGVFQHGK

SEQ ID NO: 60 - protein, 18 aa, Homo sapiens
  MOD_RES (9)..(9): Phosphorylated-Tyr
EICPGGMGYTVSGVHRRR

SEQ ID NO: 61 - protein, 10 aa, Homo sapiens
  MOD_RES (4)..(4): Phosphorylated-Tyr
KESYSVYVYK

SEQ ID NO: 62 - protein, 22 aa, Homo sapiens
  MOD_RES (16)..(16): Phosphorylated-Tyr
FNSESESGSEASSPDYFGPPAK

SEQ ID NO: 63 - protein, 9 aa, Homo sapiens
  MOD_RES (5)..(5): Phosphorylated-Tyr
SNEDYVYVR

SEQ ID NO: 64 - protein, 9 aa, Homo sapiens
  MOD_RES (5)..(5): Phosphorylated-Tyr
VGYDYDVCR

SEQ ID NO: 65 - protein, 28 aa, Homo sapiens
  MOD_RES (17)..(17): Phosphorylated-Tyr
QHPPADSSVTMEDMNEYSNIEEFAEGSK

SEQ ID NO: 66 - protein, 15 aa, Homo sapiens
  MOD_RES (3)..(3): Phosphorylated-Tyr
NIYVLQELDNPGAKR

SEQ ID NO: 67 - protein, 22 aa, Homo sapiens
  MOD_RES (13)..(13): Phosphorylated-Tyr
LGSLVDEFKELVYPPDYNPEGK

SEQ ID NO: 68 - protein, 20 aa, Homo sapiens
  MOD_RES (16)..(16): Phosphorylated-Tyr
TVDLQDAEEAVELVQYAYFK

SEQ ID NO: 69 - protein, 13 aa, Homo sapiens
  MOD_RES (6)..(6): Phosphorylated-Tyr
SQLLQYVYNLVPR

SEQ ID NO: 70 - protein, 13 aa, Homo sapiens
  MOD_RES (11)..(11): Phosphorylated-Tyr
CSILAAANPAYGR

SEQ ID NO: 71 - protein, 14 aa, Homo sapiens
  MOD_RES (10)..(10): Phosphorylated-Tyr
EAWASKDATYTSAR

SEQ ID NO: 72 - protein, 14 aa, Homo sapiens
  MOD_RES (9)..(9): Phosphorylated-Tyr
QQLAPQYTYAQGGQ

SEQ ID NO: 73 - protein, 14 aa, Homo sapiens
  MOD_RES (7)..(7): Phosphorylated-Tyr
DNGNGTYSCSYVPR

SEQ ID NO: 74 - protein, 14 aa, Homo sapiens
  MOD_RES (11)..(11): Phosphorylated-Tyr
DNGNGTYSCSYVPR

SEQ ID NO: 75 - protein, 15 aa, Homo sapiens
  MOD_RES (7)..(7): Phosphorylated-Tyr
NRMDGTYACSYTPVK

SEQ ID NO: 76 - protein, 15 aa, Homo sapiens
  MOD_RES (11)..(11): Phosphorylated-Tyr
NRMDGTYACSYTPVK

SEQ ID NO: 77 - protein, 9 aa, Homo sapiens
  MOD_RES (7)..(7): Phosphorylated-Tyr
TPCEEVYVK

SEQ ID NO: 78 - protein, 19 aa, Homo sapiens
  MOD_RES (15)..(15): Phosphorylated-Tyr
SFHEDEVDRVPHNPYATLR

SEQ ID NO: 79 - protein, 12 aa, Homo sapiens
  MOD_RES (4)..(4): Phosphorylated-Tyr
DILYVFQGIDGK

SEQ ID NO: 80 - protein, 16 aa, Homo sapiens
  MOD_RES (7)..(7): Phosphorylated-Tyr
LKQEFHYIEEDLYRTK

SEQ ID NO: 81 - protein, 31 aa, Homo sapiens
  MOD_RES (21)..(21): Phosphorylated-Tyr
GGGGGGYGSGGSSYGSGGGSYGSGGGGGGGR

SEQ ID NO: 82 - protein, 11 aa, Homo sapiens
  MOD_RES (6)..(6): Phosphorylated-Tyr
FVSTTYSGVTR

SEQ ID NO: 83 - protein, 31 aa, Homo sapiens
  MOD_RES (20)..(20): Phosphorylated-Tyr
MCGSYYGNYYGDHGYGCCGYEGLGYGYGSLR

SEQ ID NO: 84 - protein, 31 aa, Homo sapiens; identical to SEQ ID NO: 83
  MOD_RES (25)..(25): Phosphorylated-Tyr

SEQ ID NO: 85 - protein, 31 aa, Homo sapiens; identical to SEQ ID NO: 83
  MOD_RES (9)..(9): Phosphorylated-Tyr

SEQ ID NO: 86 - protein, 14 aa, Homo sapiens
  MOD_RES (4)..(4): Phosphorylated-Tyr
AQSYGGYKEPAAPV

SEQ ID NO: 87 - protein, 10 aa, Homo sapiens
  MOD_RES (3)..(3): Phosphorylated-Tyr
KPYCNAHYPK

SEQ ID NO: 88 - protein, 10 aa, Homo sapiens
  MOD_RES (8)..(8): Phosphorylated-Tyr
KPYCNAHYPK

SEQ ID NO: 89 - protein, 35 aa, Homo sapiens
  MOD_RES (21)..(21): Phosphorylated-Tyr
EGICAIGGTSEQSSVGTQHSYSEEEKYAFVNWINK

SEQ ID NO: 90 - protein, 12 aa, Homo sapiens
  MOD_RES (2)..(2): Phosphorylated-Tyr
AYYHLLEQVAPK

SEQ ID NO: 91 - protein, 22 aa, Homo sapiens
  MOD_RES (1)..(1): Phosphorylated-Tyr
YPALHKPENQDIDWGALEGETR

SEQ ID NO: 92 - protein, 19 aa, Homo sapiens
  MOD_RES (10)..(10): Phosphorylated-Tyr
GTKEDPRADYACIAENKPT

SEQ ID NO: 93 - protein, 24 aa, Homo sapiens
  MOD_RES (9)..(9): Phosphorylated-Tyr
LGIQAEPLYRVVSNTIEPLTLFHK

SEQ ID NO: 94 - protein, 7 aa, Homo sapiens
  MOD_RES (5)..(5): Phosphorylated-Tyr
AEGFYQK

SEQ ID NO: 95 - protein, 30 aa, Homo sapiens
  MOD_RES (5)..(5): Phosphorylated-Tyr
LCSQYGTPVFSAPGHALHPGEPALGEAEER

SEQ ID NO: 96 - protein, 15 aa, Homo sapiens
  MOD_RES (8)..(8): Phosphorylated-Tyr
SIEPGSDYYELSDTR

SEQ ID NO: 97 - protein, 12 aa, Homo sapiens
  MOD_RES (3)..(3): Phosphorylated-Tyr
TDYIPLLDVDEK

SEQ ID NO: 98 - protein, 14 aa, Homo sapiens
  MOD_RES (5)..(5): Phosphorylated-Tyr
LISWYDNEFGYSNR

SEQ ID NO: 99 - protein, 26 aa, Homo sapiens
  MOD_RES (11)..(11): Phosphorylated-Tyr
LISWYDNEFGYSNRVVDLMAHMASKE

SEQ ID NO: 100 - protein, 16 aa, Homo sapiens
  MOD_RES (7)..(7): Phosphorylated-Tyr
GGVSGLYDFGPVGCAL

SEQ ID NO: 101 - protein, 10 aa, Homo sapiens
  MOD_RES (3)..(3): Phosphorylated-Tyr
SCYDLSCHAR

SEQ ID NO: 102 - protein, 23 aa, Homo sapiens
  MOD_RES (3)..(3): Phosphorylated-Tyr
SAYDALPSTTIVSMACCASGSTR

SEQ ID NO: 103 - protein, 28 aa, Homo sapiens
  MOD_RES (23)..(23): Phosphorylated-Tyr
EAATQAQQTLGSTIDKATGILLYGLASR

SEQ ID NO: 104 - protein, 18 aa, Homo sapiens
  MOD_RES (13)..(13): Phosphorylated-Tyr
GFGHIGIAVPDVYSACKR

SEQ ID NO: 105 - protein, 9 aa, Homo sapiens
  MOD_RES (7)..(7): Phosphorylated-Tyr
NLNHVSYGR

SEQ ID NO: 106 - protein, 13 aa, Homo sapiens
  MOD_RES (9)..(9): Phosphorylated-Tyr
DIVHSGLAYTMER

SEQ ID NO: 107 - protein, 14 aa, Homo sapiens
  MOD_RES (3)..(3): Phosphorylated-Tyr
LHYGDLTDSTCLVK

SEQ ID NO: 108 - protein, 11 aa, Homo sapiens
  MOD_RES (6)..(6): Phosphorylated-Tyr
TLNNLYAEAEK

SEQ ID NO: 109 - protein, 9 aa, Homo sapiens
  MOD_RES (3)..(3): Phosphorylated-Tyr
HIYLLPSGR

SEQ ID NO: 110 - protein, 15 aa, Homo sapiens
  MOD_RES (4)..(4): Phosphorylated-Tyr
NLDYVATSIHEAVTK

SEQ ID NO: 111 - protein, 17 aa, Homo sapiens
  MOD_RES (6)..(6): Phosphorylated-Tyr
NLDKEYLPIGGLAEFCK

SEQ ID NO: 112 - protein, 17 aa, Homo sapiens
  MOD_RES (10)..(10): Phosphorylated-Tyr
GDVVNQDDLYQALASGK

SEQ ID NO: 113 - protein, 19 aa, Homo sapiens
  MOD_RES (8)..(8): Phosphorylated-Tyr
AFLASPEYVNLPINGNGKQ

SEQ ID NO: 114 - protein, 16 aa, Homo sapiens
  MOD_RES (9)..(9): Phosphorylated-Tyr
FQDGDLTLYQSNTILR

SEQ ID NO: 115 - protein, 9 aa, Homo sapiens
  MOD_RES (5)..(5): Phosphorylated-Tyr
FVDLYGAQK

SEQ ID NO: 116 - protein, 19 aa, Homo sapiens
  MOD_RES (16)..(16): Phosphorylated-Tyr
LHISPSNMTNQNTPEYMEK

SEQ ID NO: 117 - protein, 12 aa, Homo sapiens
  MOD_RES (10)..(10): Phosphorylated-Tyr
SIRPDNMSEYSK

SEQ ID NO: 118 - protein, 16 aa, Homo sapiens
  MOD_RES (10)..(10): Phosphorylated-Tyr
TLPFTTGLIYDSVMLK

SEQ ID NO: 119 - protein, 12 aa, Homo sapiens
  MOD_RES (8)..(8): Phosphorylated-Tyr
TNHHSCLYSAVK

SEQ ID NO: 120 - protein, 24 aa, Homo sapiens
  MOD_RES (7)..(7): Phosphorylated-Tyr
SLTKSNYQSRLHDLLYIEEIAQYK

SEQ ID NO: 121 - protein, 24 aa, Homo sapiens
  MOD_RES (16)..(16): Phosphorylated-Tyr
SLTKSNYQSRLHDLLYIEEIAQYK

SEQ ID NO: 122 - protein, 9 aa, Homo sapiens
  MOD_RES (4)..(4): Phosphorylated-Tyr
ATQYGIYER

SEQ ID NO: 123 - protein, 9 aa, Homo sapiens
  MOD_RES (7)..(7): Phosphorylated-Tyr
ATQYGIYER

SEQ ID NO: 124 - protein, 25 aa, Homo sapiens
  MOD_RES (19)..(19): Phosphorylated-Tyr
GTHMQHAYDFYKPDMLSEYPIVDGK

SEQ ID NO: 125 - protein, 13 aa, Homo sapiens
  MOD_RES (6)..(6): Phosphorylated-Tyr
NNDLCYWVPELVR

SEQ ID NO: 126 - protein, 18 aa, Homo sapiens
  MOD_RES (16)..(16): Phosphorylated-Tyr
TSSAQVEGGVHSLHSYEK

SEQ ID NO: 127 - protein, 31 aa, Homo sapiens
  MOD_RES (7)..(7): Phosphorylated-Tyr
GQQRMKYYELIVNGSYTPQTVPTGGKALSEK

SEQ ID NO: 128 - protein, 31 aa, Homo sapiens; identical to SEQ ID NO: 127
  MOD_RES (8)..(8): Phosphorylated-Tyr

SEQ ID NO: 129 - protein, 31 aa, Homo sapiens; identical to SEQ ID NO: 127
  MOD_RES (16)..(16): Phosphorylated-Tyr

SEQ ID NO: 130 - protein, 18 aa, Homo sapiens
  MOD_RES (3)..(3): Phosphorylated-Tyr
HNYECLVYVQLPFMEDLR

SEQ ID NO: 131 - protein, 15 aa, Homo sapiens
  MOD_RES (11)..(11): Phosphorylated-Tyr
QTGEGVGPQEYTLLK

SEQ ID NO: 132 - protein, 8 aa, Homo sapiens
  MOD_RES (3)..(3): Phosphorylated-Tyr
FRYLMGER

SEQ ID NO: 133 - protein, 14 aa, Homo sapiens
  MOD_RES (7)..(7): Phosphorylated-Tyr
IVADKDYSVTANSK

SEQ ID NO: 134 - protein, 15 aa, Homo sapiens
  MOD_RES (5)..(5): Phosphorylated-Tyr
VNKIYYPDFIVPDPK

SEQ ID NO: 135 - protein, 15 aa, Homo sapiens
  MOD_RES (6)..(6): Phosphorylated-Tyr
VNKIYYPDFIVPDPK

SEQ ID NO: 136 - protein, 27 aa, Homo sapiens
  MOD_RES (6)..(6): Phosphorylated-Tyr
QKCPLYEANGQAVPKEKDEMVEQEFNR

SEQ ID NO: 137 - protein, 19 aa, Homo sapiens
  MOD_RES (1)..(1): Phosphorylated-Tyr
YLRSVQLPDGGWGLHIEDK

SEQ ID NO: 138 - protein, 13 aa, Homo sapiens
  MOD_RES (1)..(1): Phosphorylated-Tyr
YQVPPDCPPLVQK

SEQ ID NO: 139 - protein, 16 aa, Homo sapiens
  MOD_RES (12)..(12): Phosphorylated-Tyr
AGAGSATLSMAYAGAR

SEQ ID NO: 140 - protein, 13 aa, Homo sapiens
  MOD_RES (2)..(2): Phosphorylated-Tyr
GYLGPEQLPDCLK

SEQ ID NO: 141 - protein, 24 aa, Homo sapiens
  MOD_RES (16)..(16): Phosphorylated-Tyr
STTTIGLVQALGAHLYQNVFACVR

SEQ ID NO: 142 - protein, 16 aa, Homo sapiens
  MOD_RES (7)..(7): Phosphorylated-Tyr
IIHEDGYSEDECKQYK

SEQ ID NO: 143 - protein, 12 aa, Homo sapiens
  MOD_RES (6)..(6): Phosphorylated-Tyr
SKIEDYFPEFAR

SEQ ID NO: 144 - protein, 17 aa, Homo sapiens
  MOD_RES (4)..(4): Phosphorylated-Tyr
HYCYPHFTCAVDTENIR

SEQ ID NO: 145 - protein, 31 aa, Homo sapiens
  MOD_RES (15)..(15): Phosphorylated-Tyr
SSWVMTCAYAPSGNYVACGGLDNICSIYNLK

SEQ ID NO: 146 - protein, 17 aa, Homo sapiens
  MOD_RES (2)..(2): Phosphorylated-Tyr
GYTSDSEVYTDHGRPGK

SEQ ID NO: 147 - protein, 17 aa, Homo sapiens
  MOD_RES (9)..(9): Phosphorylated-Tyr
GYTSDSEVYTDHGRPGK

SEQ ID NO: 148 - protein, 19 aa, Homo sapiens
  MOD_RES (3)..(3): Phosphorylated-Tyr
SPYLYPLYGLGELPQGFAR

SEQ ID NO: 149 - protein, 19 aa, Homo sapiens
  MOD_RES (5)..(5): Phosphorylated-Tyr
SPYLYPLYGLGELPQGFAR

SEQ ID NO: 150 - protein, 19 aa, Homo sapiens
  MOD_RES (8)..(8): Phosphorylated-Tyr
SPYLYPLYGLGELPQGFAR

SEQ ID NO: 151 - protein, 25 aa, Homo sapiens
  MOD_RES (5)..(5): Phosphorylated-Tyr
GGKIYKVPSTEAEALASSLMGLFEK

SEQ ID NO: 152 - protein, 22 aa, Homo sapiens
  MOD_RES (11)..(11): Phosphorylated-Tyr
YGKSPYLYPLYGLGELPQGFAR

SEQ ID NO: 153 - protein, 28 aa, Homo sapiens
  MOD_RES (8)..(8): Phosphorylated-Tyr
DRQAFSMYEPGSALKPFGGPPGDELTTR

SEQ ID NO: 154 - protein, 16 aa, Homo sapiens
  MOD_RES (7)..(7): Phosphorylated-Tyr
GHLAKIYAMHWATDSK

SEQ ID NO: 155 - protein, 12 aa, Homo sapiens
  MOD_RES (3)..(3): Phosphorylated-Tyr
ALYNIGNVYHAK

SEQ ID NO: 156 - protein, 12 aa, Homo sapiens
  MOD_RES (8)..(8): Phosphorylated-Tyr
QGVEDAFYTLVR

SEQ ID NO: 157 - protein, 12 aa, Homo sapiens
  MOD_RES (11)..(11): Phosphorylated-Tyr
IAAENELNQSYK

SEQ ID NO: 158 - protein, 17 aa, Homo sapiens
  MOD_RES (3)..(3): Phosphorylated-Tyr
IFYPETTDIYDRKNMPR

SEQ ID NO: 159 - protein, 17 aa, Homo sapiens
  MOD_RES (10)..(10): Phosphorylated-Tyr
IFYPETTDIYDRKNMPR

SEQ ID NO: 160 - protein, 24 aa, Homo sapiens
  MOD_RES (20)..(20): Phosphorylated-Tyr
LFEGENEHLSSMNNYLSETYQEFR

SEQ ID NO: 161 - protein, 23 aa, Homo sapiens
  MOD_RES (14)..(14): Phosphorylated-Tyr
TLEQTGHVSSENKYQDILNEIAK

SEQ ID NO: 162 - protein, 31 aa, Homo sapiens
  MOD_RES (28)..(28): Phosphorylated-Tyr
TLETLLLPTANISDVDPAHAQHYQDVLYHAK

SEQ ID NO: 163 - protein, 23 aa, Homo sapiens
  MOD_RES (17)..(17): Phosphorylated-Tyr
SSTSNANDIIPECADKYYDALVK

SEQ ID NO: 164 - protein, 23 aa, Homo sapiens
  MOD_RES (18)..(18): Phosphorylated-Tyr
SSTSNANDIIPECADKYYDALVK

SEQ ID NO: 165 - protein, 11 aa, Homo sapiens
  MOD_RES (4)..(4): Phosphorylated-Tyr
KYDYYYNTDSK

SEQ ID NO: 166 - protein, 11 aa, Homo sapiens
  MOD_RES (5)..(5): Phosphorylated-Tyr
KYDYYYNTDSK

SEQ ID NO: 167 - protein, 11 aa, Homo sapiens
  MOD_RES (6)..(6): Phosphorylated-Tyr
KYDYYYNTDSK

SEQ ID NO: 168 - protein, 20 aa, Homo sapiens
  MOD_RES (5)..(5): Phosphorylated-Tyr
ARDDYKTLVGSENPPLTVIR

SEQ ID NO: 169 - protein, 9 aa, Homo sapiens
  MOD_RES (3)..(3): Phosphorylated-Tyr
KIYDVEQTR

SEQ ID NO: 170 - protein, 26 aa, Homo sapiens
  MOD_RES (23)..(23): Phosphorylated-Tyr
TTVTVPNDGGPIEAVSTIETVPYWTR

SEQ ID NO: 171 - protein, 16 aa, Homo sapiens
  MOD_RES (12)..(12): Phosphorylated-Tyr
ETAVDGELVVLYDVKR

SEQ ID NO: 172 - protein, 19 aa, Homo sapiens
  MOD_RES (9)..(9): Phosphorylated-Tyr
KSEEDGKEYHFISTEEMTR

SEQ ID NO: 173 - protein, 22 aa, Homo sapiens
  MOD_RES (6)..(6): Phosphorylated-Tyr
SQYAHYFDLSLVNNGVDETLKK

SEQ ID NO: 174 - protein, 10 aa, Homo sapiens
  MOD_RES (4)..(4): Phosphorylated-Tyr
AYEYVECPIR

SEQ ID NO: 175 - protein, 19 aa, Homo sapiens
  MOD_RES (8)..(8): Phosphorylated-Tyr
VVLDDKDYFLFRDGDILGK

SEQ ID NO: 176 - protein, 12 aa, Homo sapiens
  MOD_RES (6)..(6): Phosphorylated-Tyr
GPTSYYYMLPMK

SEQ ID NO: 177 - protein, 10 aa, Homo sapiens
  MOD_RES (5)..(5): Phosphorylated-Tyr
TTTIYEEDKR

SEQ ID NO: 178 - protein, 14 aa, Homo sapiens
  MOD_RES (3)..(3): Phosphorylated-Tyr
LTYLLQNSLGGSAK

SEQ ID NO: 179 - protein, 22 aa, Homo sapiens
  MOD_RES (20)..(20): Phosphorylated-Tyr
TLGKDHPAVAATLNNLAVLYGK

SEQ ID NO: 180 - protein, 24 aa, Homo sapiens
  MOD_RES (21)..(21): Phosphorylated-Tyr
QLSNLALLCQNQGKAEEVEYYYRR

SEQ ID NO: 181 - protein, 24 aa, Homo sapiens
  MOD_RES (22)..(22): Phosphorylated-Tyr
QLSNLALLCQNQGKAEEVEYYYRR

SEQ ID NO: 182 - protein, 13 aa, Homo sapiens
  MOD_RES (5)..(5): Phosphorylated-Tyr
DSAPYGEYGSWYK

SEQ ID NO: 183 - protein, 19 aa, Homo sapiens
  MOD_RES (5)..(5): Phosphorylated-Tyr
DQNKYKDAANLLNDALAIR

SEQ ID NO: 184 - protein, 22 aa, Homo sapiens
  MOD_RES (20)..(20): Phosphorylated-Tyr
TLGKDHPAVAATLNNLAVLYGK

SEQ ID NO: 185 - protein, 24 aa, Homo sapiens
  MOD_RES (20)..(20): Phosphorylated-Tyr
QLNNLALLCQNQGKYEEVEYYYQR

SEQ ID NO: 186 - protein, 12 aa, Homo sapiens
  MOD_RES (8)..(8): Phosphorylated-Tyr
GYENEDNYSNIR

SEQ ID NO: 187 - protein, 10 aa, Homo sapiens
  MOD_RES (1)..(1): Phosphorylated-Tyr
YSEYAEEFSK

SEQ ID NO: 188 - protein, 35 aa, Homo sapiens
  MOD_RES (15)..(15): Phosphorylated-Tyr
TIEGSSPADNRYSEYAEEFSKSEPAVVSLEYGVAR

SEQ ID NO: 189 - protein, 35 aa, Homo sapiens; identical to SEQ ID NO: 188
  MOD_RES (31)..(31): Phosphorylated-Tyr

SEQ ID NO: 190 - protein, 17 aa, Homo sapiens
  MOD_RES (14)..(14): Phosphorylated-Tyr
EPCLHPLEPDEVEYEPR

SEQ ID NO: 191 - protein, 13 aa, Homo sapiens
  MOD_RES (8)..(8): Phosphorylated-Tyr
MELGRGAYGVVEK

SEQ ID NO: 192 - protein, 14 aa, Homo sapiens
  MOD_RES (10)..(10): Phosphorylated-Tyr
KNIPEGSHQYELLK

SEQ ID NO: 193 - protein, 34 aa, Homo sapiens
  MOD_RES (22)..(22): Phosphorylated-Tyr
GPPPPVGPAGSGYSGGLALAEYDQPYGGFLDILR

SEQ ID NO: 194 - protein, 11 aa, Homo sapiens
  MOD_RES (6)..(6): Phosphorylated-Tyr
VGAGTYGDVYK

SEQ ID NO: 195 - protein, 14 aa, Homo sapiens
  MOD_RES (11)..(11): Phosphorylated-Tyr
VIGNGSFGVVYQAR

SEQ ID NO: 196 - protein, 14 aa, Homo sapiens
  MOD_RES (11)..(11): Phosphorylated-Tyr
VIGNGSFGVVYQAK

SEQ ID NO: 197 - protein, 14 aa, Homo sapiens
  MOD_RES (8)..(8): Phosphorylated-Tyr
SGGSSQVYFMTLGR

SEQ ID NO: 198 - protein, 11 aa, Homo sapiens
  MOD_RES (6)..(6): Phosphorylated-Tyr
NIATYYGAFIK

SEQ ID NO: 199 - protein, 11 aa, Homo sapiens
  MOD_RES (6)..(6): Phosphorylated-Tyr
VGSGTYGDVYK

SEQ ID NO: 200 - protein, 13 aa, Homo sapiens
  MOD_RES (10)..(10): Phosphorylated-Tyr
VGSGTYGDVYKAR

SEQ ID NO: 201 - protein, 26 aa, Homo sapiens
  MOD_RES (22)..(22): Phosphorylated-Tyr
ETEPHHELPDSDGFLDSSEEIYYTAR

SEQ ID NO: 202 - protein, 23 aa, Homo sapiens
  MOD_RES (9)..(9): Phosphorylated-Tyr
SNLDLQLEYGQGHQGGYFLGANK

SEQ ID NO: 203 - protein, 23 aa, Homo sapiens
  MOD_RES (17)..(17): Phosphorylated-Tyr
SNLDLQLEYGQGHQGGYFLGANK

SEQ ID NO: 204 - protein, 14 aa, Homo sapiens
  MOD_RES (6)..(6): Phosphorylated-Tyr
VAMDDYLKNVMLER

SEQ ID NO: 205 - protein, 8 aa, Homo sapiens
  MOD_RES (3)..(3): Phosphorylated-Tyr
QYYLKQSR

SEQ ID NO: 206 - protein, 7 aa, Homo sapiens
  MOD_RES (3)..(3): Phosphorylated-Tyr
LLYDSPK

SEQ ID NO: 207 - protein, 19 aa, Homo sapiens
  MOD_RES (16)..(16): Phosphorylated-Tyr
SPSSYDNEIVMMNHVYKER

SEQ ID NO: 208 - protein, 18 aa, Homo sapiens
  MOD_RES (1)..(1): Phosphorylated-Tyr
YGAFKESVVINYTEQLLR

SEQ ID NO: 209 - protein, 24 aa, Homo sapiens
  MOD_RES (4)..(4): Phosphorylated-Tyr
AQSYPDNHQEFSDYDNPIFEKFGK

SEQ ID NO: 210 - protein, 24 aa, Homo sapiens
  MOD_RES (14)..(14): Phosphorylated-Tyr
AQSYPDNHQEFSDYDNPIFEKFGK

SEQ ID NO: 211 - protein, 12 aa, Homo sapiens
  MOD_RES (10)..(10): Phosphorylated-Tyr
YLGSASQGGYLK

SEQ ID NO: 212 - protein, 21 aa, Homo sapiens
  MOD_RES (14)..(14): Phosphorylated-Tyr
NLLNHPWIMQDYNYPVEWQSK

SEQ ID NO: 213 - protein, 16 aa, Homo sapiens
  MOD_RES (15)..(15): Phosphorylated-Tyr
QGWTTVGDMEGCGHYR

SEQ ID NO: 214 - protein, 14 aa, Homo sapiens
  MOD_RES (7)..(7): Phosphorylated-Tyr
VLGTGAYGKVFLVR

SEQ ID NO: 215 - protein, 11 aa, Homo sapiens
  MOD_RES (7)..(7): Phosphorylated-Tyr
IPQDGDYEFLK

SEQ ID NO: 216 - protein, 11 aa, Homo sapiens
  MOD_RES (10)..(10): Phosphorylated-Tyr
LGEGSYGSVYK

SEQ ID NO: 217 - protein, 19 aa, Homo sapiens
  MOD_RES (11)..(11): Phosphorylated-Tyr
LHAVVTKEPIYIITEFMAK

SEQ ID NO: 218 - protein, 12 aa, Homo sapiens
  MOD_RES (8)..(8): Phosphorylated-Tyr
LATGCAQYDPTK

SEQ ID NO: 219 - protein, 17 aa, Homo sapiens
  MOD_RES (5)..(5): Phosphorylated-Tyr
RNEEYCLLDSSEIHWWR

SEQ ID NO: 220 - protein, 12 aa, Homo sapiens
  MOD_RES (5)..(5): Phosphorylated-Tyr
TAGTYTVSVFTK

SEQ ID NO: 221 - protein, 11 aa, Homo sapiens
  MOD_RES (8)..(8): Phosphorylated-Tyr
LTADAHHYLCK

SEQ ID NO: 222 - protein, 12 aa, Homo sapiens
  MOD_RES (9)..(9): Phosphorylated-Tyr
KAGNQTGLYVLR

SEQ ID NO: 223 - protein, 12 aa, Homo sapiens
  MOD_RES (10)..(10): Phosphorylated-Tyr
RCSPKDFNKYFL

SEQ ID NO: 224 - protein, 19 aa, Homo sapiens
  MOD_RES (13)..(13): Phosphorylated-Tyr
LGAGQFGEVWMGYYNGHTK

SEQ ID NO: 225 - protein, 19 aa, Homo sapiens
  MOD_RES (14)..(14): Phosphorylated-Tyr
LGAGQFGEVWMGYYNGHTK

SEQ ID NO: 226 - protein, 19 aa, Homo sapiens
  MOD_RES (13)..(13): Phosphorylated-Tyr
FPIKWTAPEAINYGTFTIK

SEQ ID NO: 227 - protein, 7 aa, Homo sapiens
  MOD_RES (2)..(2): Phosphorylated-Tyr
LYAVVTR

SEQ ID NO: 228 - protein, 20 aa, Homo sapiens
  MOD_RES (12)..(12): Phosphorylated-Tyr
LYAVVTREEPIYIITEYMAK

SEQ ID NO: 229 - protein, 14 aa, Homo sapiens
  MOD_RES (13)..(13): Phosphorylated-Tyr
TNADVMTALSQGYR

SEQ ID NO: 230 - protein, 31 aa, Homo sapiens
  MOD_RES (20)..(20): Phosphorylated-Tyr
EKAEERPTFDYLQSVLDDFYTATEGQYQQQP

SEQ ID NO: 231 - protein, 11 aa, Homo sapiens
  MOD_RES (7)..(7): Phosphorylated-Tyr
VVEATAYGLIK

SEQ ID NO: 232 - protein, 18 aa, Homo sapiens
  MOD_RES (17)..(17): Phosphorylated-Tyr
GTEVTPETFTAGSQGSYR

SEQ ID NO: 233 - protein, 12 aa, Homo sapiens
  MOD_RES (9)..(9): Phosphorylated-Tyr
MLILIKEDYFPR

SEQ ID NO: 234 - protein, 19 aa, Homo sapiens
  MOD_RES (12)..(12): Phosphorylated-Tyr
GYGIALQHGSPYRDLFSQR

SEQ ID NO: 235 - protein, 14 aa, Homo sapiens
  MOD_RES (9)..(9): Phosphorylated-Tyr
AEPEPAEEYTEQSE

SEQ ID NO: 236 - protein, 24 aa, Homo sapiens
  MOD_RES (8)..(8): Phosphorylated-Tyr
TLKQQNQYQASYNQSFSSQPHQVE

SEQ ID NO: 237 - protein, 17 aa, Homo sapiens
  MOD_RES (4)..(4): Phosphorylated-Tyr
LVVYSLVLAAGLPLNAL

SEQ ID NO: 238 - protein, 14 aa, Homo sapiens
  MOD_RES (13)..(13): Phosphorylated-Tyr
VVAGVANALAHKYH

SEQ ID NO: 239 - protein, 10 aa, Homo sapiens
  MOD_RES (5)..(5): Phosphorylated-Tyr
LLVVYPWTQR

SEQ ID NO: 240 - protein, 26 aa, Homo sapiens
  MOD_RES (5)..(5): Phosphorylated-Tyr
KGGSYSQAASSDSAQGSDVSLTACKV

SEQ ID NO: 241 - protein, 22 aa, Homo sapiens
  MOD_RES (4)..(4): Phosphorylated-Tyr
GGSYSQAACSDSAQGSDVSLTA

SEQ ID NO: 242 - protein, 7 aa, Homo sapiens
  MOD_RES (5)..(5): Phosphorylated-Tyr
NMAKYMK

SEQ ID NO: 243 - protein, 11 aa, Homo sapiens
  MOD_RES (10)..(10): Phosphorylated-Tyr
NKAEEAMATYR

SEQ ID NO: 244 - protein, 19 aa, Homo sapiens
  MOD_RES (13)..(13): Phosphorylated-Tyr
LEGMLSQSVSSQYNMAGVR

SEQ ID NO: 245 - protein, 16 aa, Homo sapiens
  MOD_RES (8)..(8): Phosphorylated-Tyr
NSGQPPNYEMLKEEHE

SEQ ID NO: 246 - protein, 21 aa, Homo sapiens
  MOD_RES (9)..(9): Phosphorylated-Tyr
NLEDLVTEYHGNFSAWSGVSK

SEQ ID NO: 247 - protein, 13 aa, Homo sapiens
  MOD_RES (10)..(10): Phosphorylated-Tyr
GLAESLQPDYSER

SEQ ID NO: 248 - protein, 31 aa, Homo sapiens
  MOD_RES (19)..(19): Phosphorylated-Tyr
GGALGEGPGASPCNQHSPYWAPPCYTLKPET

SEQ ID NO: 249 - protein, 14 aa, Homo sapiens
  MOD_RES (3)..(3): Phosphorylated-Tyr
SPYDISNTDYFFPR

SEQ ID NO: 250 - protein, 14 aa, Homo sapiens
  MOD_RES (10)..(10): Phosphorylated-Tyr
SPYDISNTDYFFPR

SEQ ID NO: 251 - protein, 35 aa, Homo sapiens
  MOD_RES (33)..(33): Phosphorylated-Tyr
TSGGGETGPGPGLGPQGGGLPALPPSLVPLVVYSK

SEQ ID NO: 252 - protein, 10 aa, Homo sapiens
  MOD_RES (6)..(6): Phosphorylated-Tyr
LIEEAYGLDK

SEQ ID NO: 253 - protein, 18 aa, Homo sapiens
  MOD_RES (13)..(13): Phosphorylated-Tyr
AMTHVINQGMAMYWGTSR

SEQ ID NO: 254 - protein, 16 aa, Homo sapiens
  MOD_RES (11)..(11): Phosphorylated-Tyr
LHSLVKPSVDYVCQLK

SEQ ID NO: 255 - protein, 10 aa, Homo sapiens
  MOD_RES (7)..(7): Phosphorylated-Tyr
NPLQGYYQVR

SEQ ID NO: 256 - protein, 24 aa, Homo sapiens
  MOD_RES (16)..(16): Phosphorylated-Tyr
LAEGFPLPLLKRVQLYDLGLQIHK

SEQ ID NO: 257 - protein, 16 aa, Homo sapiens
  MOD_RES (12)..(12): Phosphorylated-Tyr
SPHDEDPQAVTYAEVK

SEQ ID NO: 258 - protein, 17 aa, Homo sapiens
  MOD_RES (9)..(9): Phosphorylated-Tyr
NQGAHDPDYENITLAFK

SEQ ID NO: 259 - protein, 32 aa, Homo sapiens
  MOD_RES (22)..(22): Phosphorylated-Tyr
RGGDGYDGGYGGFDDYGGYNNYGYGNDGFDDR

SEQ ID NO: 260 - protein, 20 aa, Homo sapiens
  MOD_RES (8)..(8): Phosphorylated-Tyr
GGGGSGGYYGQGGMSGGGWR

SEQ ID NO: 261 - protein, 20 aa, Homo sapiens
  MOD_RES (9)..(9): Phosphorylated-Tyr
GGGGSGGYYGQGGMSGGGWR

SEQ ID NO: 262 - protein, 21 aa, Homo sapiens
  MOD_RES (18)..(18): Phosphorylated-Tyr
GGGGPGYGNQGGGYGGGYDNY

SEQ ID NO: 263 - protein, 35 aa, Homo sapiens
  MOD_RES (35)..(35): Phosphorylated-Tyr
GNFGGSRNMGGPYGGGNYGPGGSGGSGGYGGRSRY

SEQ ID NO: 264 - protein, 32 aa, Homo sapiens
  MOD_RES (23)..(23): Phosphorylated-Tyr
MRPGAYSTGYGGYEEYSGLSDGYGFTTDLFGR

SEQ ID NO: 265 - protein, 13 aa, Homo sapiens
  MOD_RES (4)..(4): Phosphorylated-Tyr
DLSYCLSGMYDHR

SEQ ID NO: 266 - protein, 20 aa, Homo sapiens
  MOD_RES (3)..(3): Phosphorylated-Tyr
DVYLSPRDDGYSTKDSYSSR

SEQ ID NO: 267 - protein, 15 aa, Homo sapiens
  MOD_RES (4)..(4): Phosphorylated-Tyr
YDDYSSSRDGYGGSR

SEQ ID NO: 268 - protein, 29 aa, Homo sapiens
  MOD_RES (10)..(10): Phosphorylated-Tyr
GAYGGGYGGYDDYNGYNDGYGFGSDRFGR

SEQ ID NO: 269 - protein, 29 aa, Homo sapiens
  MOD_RES (16)..(16): Phosphorylated-Tyr
GAYGGGYGGYDDYNGYNDGYGFGSDRFGR

SEQ ID NO: 270 - protein, 29 aa, Homo sapiens
  MOD_RES (20)..(20): Phosphorylated-Tyr
GAYGGGYGGYDDYGGYNDGYGFGSDRFGR

SEQ ID NO: 271 - protein, 14 aa, Homo sapiens
  MOD_RES (4)..(4): Phosphorylated-Tyr
STAYEDYYYHPPPR

SEQ ID NO: 272 - protein, 29 aa, Homo sapiens
  MOD_RES (24)..(24): Phosphorylated-Tyr
NQSQGYNQWQQGQFWGQKPWSQHYHQGYY

SEQ ID NO: 273 - protein, 29 aa, Homo sapiens
  MOD_RES (29)..(29): Phosphorylated-Tyr
NQSQGYNQWQQGQFWGQKPWSQHYHQGYY

SEQ ID NO: 274 - protein, 16 aa, Homo sapiens
  MOD_RES (4)..(4): Phosphorylated-Tyr
MYSYPARVPPPPPIAR

SEQ ID NO: 275 - protein, 9 aa, Homo sapiens
  MOD_RES (3)..(3): Phosphorylated-Tyr
RDYDDMSPR

SEQ ID NO: 276 - protein, 10 aa, Homo sapiens
  MOD_RES (7)..(7): Phosphorylated-Tyr
GGDLMAYDRR

SEQ ID NO: 277 - protein, 23 aa, Homo sapiens
  MOD_RES (16)..(16): Phosphorylated-Tyr
IITITGTQDQIQNAQYLLQNSVK

SEQ ID NO: 278 - protein, 16 aa, Homo sapiens
  MOD_RES (3)..(3): Phosphorylated-Tyr
SGYAFVDYPDQNWAIR

SEQ ID NO: 279 - protein, 29 aa, Homo sapiens
  MOD_RES (20)..(20): Phosphorylated-Tyr
QAQVATGGGPGAPPGSQPDYSAAWAEYYR

SEQ ID NO: 280 - protein, 13 aa, Homo sapiens
  MOD_RES (12)..(12): Phosphorylated-Tyr
LCQIFSDLNATYR

SEQ ID NO: 281 - protein, 18 aa, Homo sapiens
  MOD_RES (6)..(6): Phosphorylated-Tyr
SQESGYYDRMDYEDDRLR

SEQ ID NO: 282 - protein, 18 aa, Homo sapiens
  MOD_RES (7)..(7): Phosphorylated-Tyr
SQESGYYDRMDYEDDRLR

SEQ ID NO: 283 - protein, 12 aa, Homo sapiens
  MOD_RES (10)..(10): Phosphorylated-Tyr
NTHCSSLPHYQK

SEQ ID NO: 284 - protein, 24 aa, Homo sapiens
  MOD_RES (14)..(14): Phosphorylated-Tyr
KLPDGMDQLPEKWYCSNNPDPQFR

SEQ ID NO: 285 - protein, 20 aa, Homo sapiens
  MOD_RES (7)..(7): Phosphorylated-Tyr
DGVNKDYEETELISTTANYR

SEQ ID NO: 286 - protein, 26 aa, Homo sapiens
  MOD_RES (7)..(7): Phosphorylated-Tyr
ISPDRVYINYYDMNAANVGWNNSTFA

SEQ ID NO: 287 - protein, 26 aa, Homo sapiens
  MOD_RES (10)..(10): Phosphorylated-Tyr
ISPDRVYINYYDMNAANVGWNNSTFA

SEQ ID NO: 288 - protein, 21 aa, Homo sapiens
  MOD_RES (4)..(4): Phosphorylated-Tyr
KGNYWTLDPACEDMFEKGNYR

SEQ ID NO: 289 - protein, 14 aa, Homo sapiens
  MOD_RES (12)..(12): Phosphorylated-Tyr
GLAGPAASYGPYTR

SEQ ID NO: 290 - protein, 16 aa, Homo sapiens
  MOD_RES (6)..(6): Phosphorylated-Tyr
DRTGHYLCNACGLYHK

SEQ ID NO: 291 - protein, 16 aa, Homo sapiens
  MOD_RES (14)..(14): Phosphorylated-Tyr
NASGDPVCNACGLYYK

SEQ ID NO: 292 - protein, 30 aa, Homo sapiens
  MOD_RES (8)..(8): Phosphorylated-Tyr
HSPVFQVYPLLNCMEGIPGGSPYAGWAYGK

SEQ ID NO: 293 - protein, 16 aa, Homo sapiens
  MOD_RES (6)..(6): Phosphorylated-Tyr
DGTGHYLCNACGLYHK

SEQ ID NO: 294 - protein, 16 aa, Homo sapiens
  MOD_RES (14)..(14): Phosphorylated-Tyr
DGTGHYLCNACGLYHK

SEQ ID NO: 295 - protein, 14 aa, Homo sapiens
  MOD_RES (5)..(5): Phosphorylated-Tyr
GEFTYYEIQDNTGK

SEQ ID NO: 296 - protein, 15 aa, Homo sapiens
  MOD_RES (7)..(7): Phosphorylated-Tyr
GLSDTPYDSSASYEK

SEQ ID NO: 297 - protein, 21 aa, Homo sapiens
  MOD_RES (9)..(9): Phosphorylated-Tyr
SSAQLQTNYPSSDNSLYTNAK

SEQ ID NO: 298 - protein, 15 aa, Homo sapiens
  MOD_RES (10)..(10): Phosphorylated-Tyr
AQILDKATEYIQYMR

SEQ ID NO: 299 - protein, 31 aa, Homo sapiens
  MOD_RES (30)..(30): Phosphorylated-Tyr
WRQCLQFAMKRLLPSVWSESEDGAGSPPPYR

SEQ ID NO: 300 - protein, 25 aa, Homo sapiens
  MOD_RES (17)..(17): Phosphorylated-Tyr
FGPYVGEQRSNLKDPSYGWEVHLPR

SEQ ID NO: 301 - protein, 26 aa, Homo sapiens
  MOD_RES (18)..(18): Phosphorylated-Tyr
FSPPEPGDTPTDEPDALYVACQGQPK

SEQ ID NO: 302 - protein, 18 aa, Homo sapiens
  MOD_RES (4)..(4): Phosphorylated-Tyr
VGDYVYFENSSSNPYLIR

SEQ ID NO: 303 - protein, 18 aa, Homo sapiens
  MOD_RES (6)..(6): Phosphorylated-Tyr
VGDYVYFENSSSNPYLIR

SEQ ID NO: 304 - protein, 18 aa, Homo sapiens
  MOD_RES (4)..(4): Phosphorylated-Tyr
VGDYVYFENSSSNPYLVR

SEQ ID NO: 305 - protein, 16 aa, Homo sapiens
  MOD_RES (3)..(3): Phosphorylated-Tyr
SGYAFVDCPDEHWAMK

SEQ ID NO: 306 - protein, 12 aa, Homo sapiens
  MOD_RES (8)..(8): Phosphorylated-Tyr
FIEDLNMYEASK

SEQ ID NO: 307 - protein, 22 aa, Homo sapiens
  MOD_RES (7)..(7): Phosphorylated-Tyr
CAECTNYDLCTVCYHGDKHHLR

SEQ ID NO: 308 - protein, 25 aa, Homo sapiens
  MOD_RES (17)..(17): Phosphorylated-Tyr
VTEIQDWSASSPHSAAYVLWDNGAK

SEQ ID NO: 309 - protein, 23 aa, Homo sapiens
  MOD_RES (4)..(4): Phosphorylated-Tyr
MQAYSNPGYSSFPSPTGLEPSCK

SEQ ID NO: 310 - protein, 23 aa, Homo sapiens
  MOD_RES (9)..(9): Phosphorylated-Tyr
MQAYSNPGYSSFPSPTGLEPSCK

SEQ ID NO: 311 - protein, 24 aa, Homo sapiens
  MOD_RES (9)..(9): Phosphorylated-Tyr
SPYTLDSLYWSVKPASSSFGSEAK

SEQ ID NO: 312 - protein, 25 aa, Homo sapiens
  MOD_RES (15)..(15): Phosphorylated-Tyr
DDDEYIKFLDLFLSYILERDLPYSR

SEQ ID NO: 313 - protein, 25 aa, Homo sapiens
  MOD_RES (23)..(23): Phosphorylated-Tyr
DDDEYIKFLDLFLSYILERDLPYSR

SEQ ID NO: 314 - protein, 18 aa, Homo sapiens
  MOD_RES (11)..(11): Phosphorylated-Tyr
QKLKDLEQMLYKATVNAR

SEQ ID NO: 315 - protein, 19 aa, Homo sapiens
  MOD_RES (2)..(2): Phosphorylated-Tyr
LYAHVYGNGQSEKPDENEK

SEQ ID NO: 316 - protein, 17 aa, Homo sapiens
  MOD_RES (7)..(7): Phosphorylated-Tyr
RFLLPEYLPYAGIFHER

SEQ ID NO: 317 - protein, 13 aa, Homo sapiens
  MOD_RES (6)..(6): Phosphorylated-Tyr
IAAYAYSALSQIR

SEQ ID NO: 318 - protein, 14 aa, Homo sapiens
  MOD_RES (8)..(8): Phosphorylated-Tyr
IQELGDLYTPAPGR

SEQ ID NO: 319 - protein, 13 aa, Homo sapiens
  MOD_RES (10)..(10): Phosphorylated-Tyr
AAVFEEIDTYERR

SEQ ID NO: 320 - protein, 17 aa, Homo sapiens
  MOD_RES (15)..(15): Phosphorylated-Tyr
YRHDENILESEPIVYRR

SEQ ID NO: 321 - protein, 16 aa, Homo sapiens
  MOD_RES (9)..(9): Phosphorylated-Tyr
DEEDEDESYQSALANK

SEQ ID NO: 322 - protein, 11 aa, Homo sapiens
  MOD_RES (4)..(4): Phosphorylated-Tyr
IKSYDYEAWAK

SEQ ID NO: 323 - protein, 11 aa, Homo sapiens
  MOD_RES (6)..(6): Phosphorylated-Tyr
IKSYDYEAWAK

SEQ ID NO: 324 - protein, 23 aa, Homo sapiens
  MOD_RES (10)..(10): Phosphorylated-Tyr
SISDAPAPAYHDPLYLEDQVSHR

SEQ ID NO: 325 - protein, 23 aa, Homo sapiens
  MOD_RES (15)..(15): Phosphorylated-Tyr
SISDAPAPAYHDPLYLEDQVSHR

SEQ ID NO: 326 - protein, 9 aa, Homo sapiens
  MOD_RES (1)..(1): Phosphorylated-Tyr
YIMRYNNYK

SEQ ID NO: 327 - protein, 9 aa, Homo sapiens
  MOD_RES (5)..(5): Phosphorylated-Tyr
YIMRYNNYK

SEQ ID NO: 328 - protein, 10 aa, Homo sapiens
  MOD_RES (4)..(4): Phosphorylated-Tyr
IEHYFSPVSK

SEQ ID NO: 329 - protein, 25 aa, Homo sapiens
  MOD_RES (8)..(8): Phosphorylated-Tyr
YSYFEPRYILVVPMNKEKYEGYLRR

SEQ ID NO: 330 - protein, 25 aa, Homo sapiens
  MOD_RES (19)..(19): Phosphorylated-Tyr
YSYFEPRYILVVPMNKEKYEGYLRR

SEQ ID NO: 331 - protein, 25 aa, Homo sapiens
  MOD_RES (22)..(22): Phosphorylated-Tyr
YSYFEPRYILVVPMNKEKYEGYLRR

SEQ ID NO: 332 - protein, 20 aa, Homo sapiens
  MOD_RES (16)..(16): Phosphorylated-Tyr
REPQEQPNLLQQLLQYTVSK

SEQ ID NO: 333 - protein, 18 aa, Homo sapiens
  MOD_RES (15)..(15): Phosphorylated-Tyr
PICSFIGLTLANNTYVQK

SEQ ID NO: 334 - protein, 16 aa, Homo sapiens
  MOD_RES (5)..(5): Phosphorylated-Tyr
LEHAYKPVQFEGSLGK

SEQ ID NO: 335 - protein, 11 aa, Homo sapiens
  MOD_RES (5)..(5): Phosphorylated-Tyr
ICSMYDNLRGK

SEQ ID NO: 336 - protein, 14 aa, Homo sapiens
  MOD_RES (8)..(8): Phosphorylated-Tyr
AADQFDIYSSQQSK

SEQ ID NO: 337 - protein, 15 aa, Homo sapiens
  MOD_RES (1)..(1): Phosphorylated-Tyr
YDIDDENEIDENDVK

SEQ ID NO: 338 - protein, 7 aa, Homo sapiens
  MOD_RES (6)..(6): Phosphorylated-Tyr
SNTRAYY

SEQ ID NO: 339 - protein, 23 aa, Homo sapiens
  MOD_RES (3)..(3): Phosphorylated-Tyr
KLYECKECGKSYYSSGSFLNHKR

SEQ ID NO: 340 - protein, 23 aa, Homo sapiens
  MOD_RES (12)..(12): Phosphorylated-Tyr
KLYECKECGKSYYSSGSFLNHKR

SEQ ID NO: 341 - protein, 23 aa, Homo sapiens
  MOD_RES (13)..(13): Phosphorylated-Tyr
KLYECKECGKSYYSSGSFLNHKR

SEQ ID NO: 342 - protein, 21 aa, Homo sapiens
  MOD_RES (6)..(6): Phosphorylated-Tyr
RQSGLYDSQNPPTVNNCAQDR

SEQ ID NO: 343 - protein, 29 aa, Homo sapiens
  MOD_RES (19)..(19): Phosphorylated-Tyr
LISDKIGSLGLGTGEDDDYVDDFNSTSHR

SEQ ID NO: 344 - protein, 12 aa, Homo sapiens
  MOD_RES (6)..(6): Phosphorylated-Tyr
FEDGGYVVCNTR

SEQ ID NO: 345 - protein, 26 aa, Homo sapiens
  MOD_RES (14)..(14): Phosphorylated-Tyr
VLCTRPSGNSAEEYYENVPCKAERPR

SEQ ID NO: 346 - protein, 21 aa, Homo sapiens
  MOD_RES (10)..(10): Phosphorylated-Tyr
ESLGGTETEYSLLHMPSTDPR

SEQ ID NO: 347 - protein, 13 aa, Homo sapiens
  MOD_RES (6)..(6): Phosphorylated-Tyr
SPEDEYELLMPHR

SEQ ID NO: 348 - protein, 26 aa, Homo sapiens
  MOD_RES (14)..(14): Phosphorylated-Tyr
MSSTPIQDNVDQTYSEELCYTLINHR

SEQ ID NO: 349 - protein, 26 aa, Homo sapiens
  MOD_RES (20)..(20): Phosphorylated-Tyr
MSSTPIQDNVDQTYSEELCYTLINHR

SEQ ID NO: 350 - protein, 26 aa, Homo sapiens
  MOD_RES (20)..(20): Phosphorylated-Tyr
VVFAVSDLEEGTYQKLLPLYFPRSLK

SEQ ID NO: 351 - protein, 21 aa, Homo sapiens
  MOD_RES (15)..(15): Phosphorylated-Tyr
MVQCNEGAYFSDDIYKDTEER

SEQ ID NO: 352 - protein, 19 aa, Homo sapiens
  MOD_RES (16)..(16): Phosphorylated-Tyr
LMDNIDQAQLDWELIYIGR

SEQ ID NO: 353 - protein, 14 aa, Homo sapiens
  MOD_RES (9)..(9): Phosphorylated-Tyr
SVTRPAFLYNPLNK

SEQ ID NO: 354 - protein, 9 aa, Homo sapiens
  MOD_RES (4)..(4): Phosphorylated-Tyr
NALYESQLK

SEQ ID NO: 355 - protein, 11 aa, Homo sapiens
  MOD_RES (5)..(5): Phosphorylated-Tyr
IEAGYIQTGDR

SEQ ID NO: 356 - protein, 35 aa, Homo sapiens
  MOD_RES (11)..(11): Phosphorylated-Tyr
DKPSVEPVEEYDYEDLKESSNSVSNHQLSGFDQAR

SEQ ID NO: 357 - protein, 35 aa, Homo sapiens; identical to SEQ ID NO: 356
  MOD_RES (13)..(13): Phosphorylated-Tyr

SEQ ID NO: 358 - protein, 11 aa, Homo sapiens
  MOD_RES (2)..(2): Phosphorylated-Tyr
FYHEELNAPIR

SEQ ID NO: 359 - protein, 11 aa, Homo sapiens
  MOD_RES (7)..(7): Phosphorylated-Tyr
ATDDYHYEKFK

SEQ ID NO: 360 - protein, 21 aa, Homo sapiens
  MOD_RES (1)..(1): Phosphorylated-Tyr
YLDEADRDKERYMKELEQYQK

SEQ ID NO: 361 - protein, 21 aa, Homo sapiens
  MOD_RES (12)..(12): Phosphorylated-Tyr
YLDEADRDKERYMKELEQYQK

SEQ ID NO: 362 - protein, 21 aa, Homo sapiens
  MOD_RES (19)..(19): Phosphorylated-Tyr
YLDEADRDKERYMKELEQYQK

SEQ ID NO: 363 - protein, 11 aa, Homo sapiens
  MOD_RES (8)..(8): Phosphorylated-Tyr
VPNGSNPYTLK

SEQ ID NO: 364 - protein, 10 aa, Homo sapiens
  MOD_RES (8)..(8): Phosphorylated-Tyr
SRDEGYQYHR

SEQ ID NO: 365 - protein, 14 aa, Homo sapiens
  MOD_RES (6)..(6): Phosphorylated-Tyr
LSEVIYEPFQLLSK

SEQ ID NO: 366 - protein, 39 aa, Homo sapiens
  MOD_RES (4)..(4): Phosphorylated-Tyr
EDLYLEPQVGHDPAGPAASPVLADGLSVSQAPAILPVSK

SEQ ID NO: 367 - protein, 12 aa, Homo sapiens
  MOD_RES (10)..(10): Phosphorylated-Tyr
EAQSIQYDPYSK

SEQ ID NO: 368 - protein, 13 aa, Homo sapiens
  MOD_RES (11)..(11): Phosphorylated-Tyr
QAIELSPDNQYVK

SEQ ID NO: 369 - protein, 12 aa, Homo sapiens
  MOD_RES (10)..(10): Phosphorylated-Tyr
AVTLNPDNSYIK

SEQ ID NO: 370 - protein, 11 aa, Homo sapiens
  MOD_RES (7)..(7): Phosphorylated-Tyr
MNMGNIYLKQR

SEQ ID NO: 371 - protein, 16 aa, Homo sapiens
  MOD_RES (4)..(4): Phosphorylated-Tyr
SGNYQKALDTYKDTHR

SEQ ID NO: 372 - protein, 16 aa, Homo sapiens
  MOD_RES (11)..(11): Phosphorylated-Tyr
SGNYQKALDTYKDTHR

SEQ ID NO: 373 - protein, 32 aa, Homo sapiens
  MOD_RES (5)..(5): Phosphorylated-Tyr
SESDYSDGDNDSINSTSNSNDTINCSSESSSR

SEQ ID NO: 374 - protein, 15 aa, Homo sapiens
  MOD_RES (13)..(13): Phosphorylated-Tyr
KKDFPCDQSINPYLK

SEQ ID NO: 375 - protein, 13 aa, Homo sapiens
  MOD_RES (4)..(4): Phosphorylated-Tyr
EAEYFELPELVRR

SEQ ID NO: 376 - protein, 19 aa, Homo sapiens
  MOD_RES (16)..(16): Phosphorylated-Tyr
KMLRHAEASAIVEYAYNDK

SEQ ID NO: 377 - protein, 8 aa, Homo sapiens
  MOD_RES (5)..(5): Phosphorylated-Tyr
IDVSYEYR

SEQ ID NO: 378 - protein, 25 aa, Homo sapiens
  MOD_RES (8)..(8): Phosphorylated-Tyr
TDLHNEGYILELDCCSSLDHPTDQK

SEQ ID NO: 379 - protein, 12 aa, Homo sapiens
  MOD_RES (3)..(3): Phosphorylated-Tyr
AIYQVYNALQEK

SEQ ID NO: 380 - protein, 31 aa, Homo sapiens
  MOD_RES (15)..(15): Phosphorylated-Tyr
STLGDAEASDPPPPYSDFHPNNQESTLSHSR

SEQ ID NO: 381 - protein, 36 aa, Homo sapiens
  MOD_RES (15)..(15): Phosphorylated-Tyr
SVFMPRPQAVGSSNYASTSAGLKYPGSGADLPPPQR

SEQ ID NO: 382 - protein, 13 aa, Homo sapiens
  MOD_RES (5)..(5): Phosphorylated-Tyr
EIADYLAAGKDER

SEQ ID NO: 383 - protein, 15 aa, Homo sapiens
  MOD_RES (6)..(6): Phosphorylated-Tyr
APDPAYIYDEFLQQR

SEQ ID NO: 384 - protein, 9 aa, Homo sapiens
  MOD_RES (6)..(6): Phosphorylated-Tyr
GQSVKYVYK

SEQ ID NO: 385 - protein, 10 aa, Homo sapiens
  MOD_RES (2)..(2): Phosphorylated-Tyr
TYQASSAAFR

SEQ ID NO: 386 - protein, 28 aa, Homo sapiens
  MOD_RES (9)..(9): Phosphorylated-Tyr
SHNVAAGSYEAPLPPGSFHGDFAYGTYR

SEQ ID NO: 387 - protein, 28 aa, Homo sapiens
  MOD_RES (24)..(24): Phosphorylated-Tyr
SHNVAAGSYEAPLPPGSFHGDFAYGTYR

SEQ ID NO: 388 - protein, 11 aa, Homo sapiens
  MOD_RES (1)..(1): Phosphorylated-Tyr
YGPLPGPAVPR

SEQ ID NO: 389 - protein, 29 aa, Homo sapiens
  MOD_RES (26)..(26): Phosphorylated-Tyr
HGAVCHTGAPDATLHTVHPDSVSSSYSSR

SEQ ID NO: 390 - protein, 21 aa, Homo sapiens
  MOD_RES (20)..(20): Phosphorylated-Tyr
QIDSSPVGGETDETTVSQNYR

SEQ ID NO: 391 - protein, 20 aa, Homo sapiens
  MOD_RES (11)..(11): Phosphorylated-Tyr
ANHSSHQEDTYGALDFTLSR

SEQ ID NO: 392 - protein, 28 aa, Homo sapiens
  MOD_RES (9)..(9): Phosphorylated-Tyr
TLENPVNVYNPSHSDSLASQQSVASHPR

SEQ ID NO: 393 - protein, 7 aa, Homo sapiens
  MOD_RES (2)..(2): Phosphorylated-Tyr
FYQQVTK

SEQ ID NO: 394 - protein, 16 aa, Homo sapiens
  MOD_RES (1)..(1): Phosphorylated-Tyr
YRPYDGAASAYAQNYR

SEQ ID NO: 395 - protein, 16 aa, Homo sapiens
  MOD_RES (4)..(4): Phosphorylated-Tyr
YRPYDGAASAYAQNYR

SEQ ID NO: 396 - protein, 16 aa, Homo sapiens
  MOD_RES (11)..(11): Phosphorylated-Tyr
YRPYDGAASAYAQNYR

SEQ ID NO: 397 - protein, 22 aa, Homo sapiens
  MOD_RES (18)..(18): Phosphorylated-Tyr
HIVIDGSNVAMVHGLQHYFSSR

SEQ ID NO: 398 - protein, 15 aa, Homo sapiens
  MOD_RES (8)..(8): Phosphorylated-Tyr
THTGEKPYECLECGK

SEQ ID NO: 399 - protein, 37 aa, Homo sapiens
  MOD_RES (17)..(17): Phosphorylated-Tyr
NAIPTKIQKGFRSIVVYLTALDTNGDYIAVGSSIGML

SEQ ID NO: 400 - protein, 13 aa, Homo sapiens
  MOD_RES (8)..(8): Phosphorylated-Tyr
CLLTSAIYALQGR

SEQ ID NO: 401 - protein, 12 aa, Homo sapiens
  MOD_RES (7)..(7): Phosphorylated-Tyr
NNGIVSYDLLVK

SEQ ID NO: 402 - protein, 24 aa, Homo sapiens
  MOD_RES (16)..(16): Phosphorylated-Tyr
NGPSLTEALENAGIFYEAQYKEVK

SEQ ID NO: 403 - protein, 36 aa, Homo sapiens
  MOD_RES (20)..(20): Phosphorylated-Tyr
NGPSLTEALENAGIFYEAQYKEVKVVANAYKTFANR

SEQ ID NO: 404 - protein, 14 aa, Homo sapiens
  MOD_RES (6)..(6): Phosphorylated-Tyr
SQEPIYSEEASGPR

SEQ ID NO: 405 - protein, 12 aa, Homo sapiens
  MOD_RES (11)..(11): Phosphorylated-Tyr
QQQQQQQEQLYK

SEQ ID NO: 406 - protein, 12 aa, Homo sapiens
  MOD_RES (7)..(7): Phosphorylated-Tyr
RDPSLPYLEQYR

SEQ ID NO: 407 - protein, 25 aa, Homo sapiens
  MOD_RES (12)..(12): Phosphorylated-Tyr
AGATASKEPPLYYGVCPVYEDVPAR

SEQ ID NO: 408 - protein, 25 aa, Homo sapiens
  MOD_RES (13)..(13): Phosphorylated-Tyr
AGATASKEPPLYYGVCPVYEDVPAR

SEQ ID NO: 409 - protein, 25 aa, Homo sapiens
  MOD_RES (19)..(19): Phosphorylated-Tyr
AGATASKEPPLYYGVCPVYEDVPAR

SEQ ID NO: 410 - protein, 20 aa, Homo sapiens
  MOD_RES (7)..(7): Phosphorylated-Tyr
KLPTSPYPCVFCCEEETQQR

SEQ ID NO: 411 - protein, 13 aa, Homo sapiens
  MOD_RES (3)..(3): Phosphorylated-Tyr
QDYCSICLQQYKR

SEQ ID NO: 412 - protein, 20 aa, Homo sapiens
  MOD_RES (13)..(13): Phosphorylated-Tyr
GSQIDSHSSNSNYHDSWETR

SEQ ID NO: 413 - protein, 16 aa, Homo sapiens
  MOD_RES (8)..(8): Phosphorylated-Tyr
AKPEPPKYGIVQEFFR

SEQ ID NO: 414 - protein, 23 aa, Homo sapiens
  MOD_RES (10)..(10): Phosphorylated-Tyr
GGMNDDEDFYDEDMGDGGGGSYR

SEQ ID NO: 415 - protein, 12 aa, Homo sapiens
  MOD_RES (1)..(1): Phosphorylated-Tyr
YMVRRYFNKPSR

SEQ ID NO: 416 - protein, 12 aa, Homo sapiens
  MOD_RES (6)..(6): Phosphorylated-Tyr
YMVRRYFNKPSR

SEQ ID NO: 417 - protein, 33 aa, Homo sapiens
  MOD_RES (4)..(4): Phosphorylated-Tyr
WRSYKGELFDKTPVLLTDYPVDLDEIAYFTWAS

SEQ ID NO: 418 - protein, 33 aa, Homo sapiens; identical to SEQ ID NO: 417
  MOD_RES (19)..(19): Phosphorylated-Tyr

SEQ ID NO: 419 - protein, 15 aa, Homo sapiens
  MOD_RES (13)..(13): Phosphorylated-Tyr
NLIAFSEDGSDPYVR

SEQ ID NO: 420 - protein, 14 aa, Homo sapiens
  MOD_RES (12)..(12): Phosphorylated-Tyr
NQQLEAIAKEHYER

SEQ ID NO: 421 - protein, 15 aa, Homo sapiens
  MOD_RES (15)..(15): Phosphorylated-Tyr
TYGSMKDDSWKDGCY

SEQ ID NO: 422 - protein, 24 aa, Homo sapiens
  MOD_RES (9)..(9): Phosphorylated-Tyr
SSSLEMTPYNTPQLSPATTPANKK

SEQ ID NO: 423 - protein, 28 aa, Homo sapiens
  MOD_RES (11)..(11): Phosphorylated-Tyr
TSPSDGAMANYESTEVMGDGESAHDSPR

SEQ ID NO: 424 - protein, 20 aa, Homo sapiens
  MOD_RES (19)..(19): Phosphorylated-Tyr
PDIMIILLSKLMEEGDMFYK

SEQ ID NO: 425 - protein, 19 aa, Homo sapiens
  MOD_RES (5)..(5): Phosphorylated-Tyr
PSSAYRGGVRYSQTPQIGR

SEQ ID NO: 426 - protein, 13 aa, Homo sapiens
  MOD_RES (6)..(6): Phosphorylated-Tyr
SQSASYYPVCHSK

SEQ ID NO: 427 - protein, 26 aa, Homo sapiens
  MOD_RES (14)..(14): Phosphorylated-Tyr
TNNAQNGHLLEDDYYSPHGMLANGSR

SEQ ID NO: 428 - protein, 26 aa, Homo sapiens
  MOD_RES (15)..(15): Phosphorylated-Tyr
TNNAQNGHLLEDDYYSPHGMLANGSR

SEQ ID NO: 429 - protein, 12 aa, Homo sapiens
  MOD_RES (6)..(6): Phosphorylated-Tyr
QEVPMYTGPESR

SEQ ID NO: 430 - protein, 16 aa, Homo sapiens
  MOD_RES (12)..(12): Phosphorylated-Tyr
STSQLVNLQPDYINPR

SEQ ID NO: 431 - protein, 13 aa, Homo sapiens
  MOD_RES (3)..(3): Phosphorylated-Tyr
DIYSTVIDIHTLR

SEQ ID NO: 432 - protein, 28 aa, Homo sapiens
  MOD_RES (25)..(25): Phosphorylated-Tyr
TAADELEAFLGGGAPGGRHPGGGDYEEL

SEQ ID NO: 433 - protein, 23 aa, Homo sapiens
  MOD_RES (10)..(10): Phosphorylated-Tyr
CDTIDTREEYEMKDPTNGYYNVR

SEQ ID NO: 434 - protein, 23 aa, Homo sapiens
  MOD_RES (19)..(19): Phosphorylated-Tyr
CDTIDTREEYEMKDPTNGYYNVR

SEQ ID NO: 435 - protein, 23 aa, Homo sapiens
  MOD_RES (20)..(20): Phosphorylated-Tyr
CDTIDTREEYEMKDPTNGYYNVR

SEQ ID NO: 436 - protein, 17 aa, Homo sapiens
  MOD_RES (12)..(12): Phosphorylated-Tyr
TPYEAYDPIGKYATATR

SEQ ID NO: 437 - protein, 22 aa, Homo sapiens
  MOD_RES (9)..(9): Phosphorylated-Tyr
SVLAAATEYFTPLLSGQFSESR

SEQ ID NO: 438 - protein, 16 aa, Homo sapiens
  MOD_RES (11)..(11): Phosphorylated-Tyr
HHEGGTPYAEYGGWYK

SEQ ID NO: 439 - protein, 15 aa, Homo sapiens
  MOD_RES (13)..(13): Phosphorylated-Tyr
ETSTLQVEQNGDYGR

SEQ ID NO: 440 - protein, 14 aa, Homo sapiens
  MOD_RES (6)..(6): Phosphorylated-Tyr
EYEVMYSSSCETTR

SEQ ID NO: 441 - protein, 17 aa, Homo sapiens
  MOD_RES (13)..(13): Phosphorylated-Tyr
LIIVSNPVDILTYVAWK

SEQ ID NO: 442 - protein, 34 aa, Homo sapiens
  MOD_RES (20)..(20): Phosphorylated-Tyr
AEPWLSQPASGSAYATPGAYGDIRPSAASWVGSR

SEQ ID NO: 443 - protein, 34 aa, Homo sapiens; identical to SEQ ID NO: 442
  MOD_RES (14)..(14): Phosphorylated-Tyr

SEQ ID NO: 444 - protein, 42 aa, Homo sapiens
  MOD_RES (40)..(40): Phosphorylated-Tyr
ALPAAAATAGCAGLEATYSNVGLAALPGVSLAASPVVAEYAR

SEQ ID NO: 445 - protein, 24 aa, Homo sapiens
  MOD_RES (22)..(22): Phosphorylated-Tyr
SPQEPQQGKTEVTPAAQVDVLYSR

SEQ ID NO: 446 - protein, 14 aa, Homo sapiens
  MOD_RES (4)..(4): Phosphorylated-Tyr
LKSYSMPISIEFQR

SEQ ID NO: 447 - protein, 19 aa, Homo sapiens
  MOD_RES (15)..(15): Phosphorylated-Tyr
CPCCPDKCCCPEALYAAGK

SEQ ID NO: 448 - protein, 22 aa, Homo sapiens
  MOD_RES (10)..(10): Phosphorylated-Tyr
AATSGVPSIYAPSTYAHLSPAK

SEQ ID NO: 449 - protein, 22 aa, Homo sapiens
  MOD_RES (15)..(15): Phosphorylated-Tyr
AATSGVPSIYAPSTYAHLSPAK

SEQ ID NO: 450 - protein, 12 aa, Homo sapiens
  MOD_RES (7)..(7): Phosphorylated-Tyr
VGCFDPYSDDPR

SEQ ID NO: 451 - protein, 12 aa, Homo sapiens
  MOD_RES (8)..(8): Phosphorylated-Tyr
STTELDDYSTNK

SEQ ID NO: 452 - protein, 12 aa, Homo sapiens
  MOD_RES (6)..(6): Phosphorylated-Tyr
LFQKIYGENGSK

SEQ ID NO: 453 - protein, 8 aa, Homo sapiens
  MOD_RES (4)..(4): Phosphorylated-Tyr
GGQYENFR

SEQ ID NO: 454 - protein, 13 aa, Homo sapiens
  MOD_RES (3)..(3): Phosphorylated-Tyr
VQYTETEPYHNYR

SEQ ID NO: 455 - protein, 13 aa, Homo sapiens
  MOD_RES (9)..(9): Phosphorylated-Tyr
VQYTETEPYHNYR

SEQ ID NO: 456 - protein, 15 aa, Homo sapiens
  MOD_RES (11)..(11): Phosphorylated-Tyr
ANGSVSLQDMYGQEK

SEQ ID NO: 457 - protein, 13 aa, Homo sapiens
  MOD_RES (8)..(8): Phosphorylated-Tyr
DGTVWQLYEWQQR

SEQ ID NO: 458 - protein, 21 aa, Homo sapiens
  MOD_RES (19)..(19): Phosphorylated-Tyr
NGMLPASYGPGEQNGTGGYQR

SEQ ID NO: 459 - protein, 18 aa, Homo sapiens
  MOD_RES (13)..(13): Phosphorylated-Tyr
QGPGQSLSFPENYQTLPK

SEQ ID NO: 460 - protein, 33 aa, Homo sapiens
  MOD_RES (23)..(23): Phosphorylated-Tyr
SQEVSSTSNQENENGSGSEEVCYTVINHIPHQR

SEQ ID NO: 461 - protein, 18 aa, Homo sapiens
  MOD_RES (10)..(10): Phosphorylated-Tyr
SSLSSNDDGYENIDSLTR

SEQ ID NO: 462 - protein, 10 aa, Homo sapiens
  MOD_RES (1)..(1): Phosphorylated-Tyr
YQPYKVIKQK

SEQ ID NO: 463 - protein, 16 aa, Homo sapiens
  MOD_RES (11)..(11): Phosphorylated-Tyr
RTQTCTEGGDYCLIPR

SEQ ID NO: 464-465 - skipped (2 entries)

SEQ ID NO: 466 - protein, 27 aa, Homo sapiens
  MOD_RES (1)..(1): Phosphorylated-Tyr
YDIGGKYSHLPYNKYSVLLPLVAKEGK

SEQ ID NO: 467 - protein, 27 aa, Homo sapiens
  MOD_RES (7)..(7): Phosphorylated-Tyr
YDIGGKYSHLPYNKYSVLLPLVAKEGK

SEQ ID NO: 468 - protein, 27 aa, Homo sapiens
  MOD_RES (12)..(12): Phosphorylated-Tyr
YDIGGKYSHLPYNKYSVLLPLVAKEGK

SEQ ID NO: 469 - protein, 27 aa, Homo sapiens
  MOD_RES (15)..(15): Phosphorylated-Tyr
YDIGGKYSHLPYNKYSVLLPLVAKEGK

SEQ ID NO: 470 - protein, 14 aa, Homo sapiens
  MOD_RES (5)..(5): Phosphorylated-Tyr
ELVPYDIFTEKYMK

SEQ ID NO: 471 - protein, 15 aa, Homo sapiens
  MOD_RES (1)..(1): Phosphorylated-Tyr
YLLQNTALEIFMANR

SEQ ID NO: 472 - protein, 16 aa, Homo sapiens
  MOD_RES (5)..(5): Phosphorylated-Tyr
LIASYCNVGDIEGASK

SEQ ID NO: 473 - protein, 30 aa, Homo sapiens
  MOD_RES (12)..(12): Phosphorylated-Tyr
TKSEFNFSSKTYQEFNYYLTSMVGCLWTSK

SEQ ID NO: 474 - protein, 30 aa, Homo sapiens; identical to SEQ ID NO: 473
  MOD_RES (17)..(17): Phosphorylated-Tyr

SEQ ID NO: 475 - protein, 30 aa, Homo sapiens; identical to SEQ ID NO: 473
  MOD_RES (18)..(18): Phosphorylated-Tyr

SEQ ID NO: 476 - protein, 15 aa, Homo sapiens
  MOD_RES (5)..(5): Phosphorylated-Tyr
SDKQYAENYTRPSSR

SEQ ID NO: 477 - protein, 12 aa, Homo sapiens
  MOD_RES (8)..(8): Phosphorylated-Tyr
LAEQVSSYNESK

SEQ ID NO: 478 - protein, 13 aa, Homo sapiens
  MOD_RES (10)..(10): Phosphorylated-Tyr
NVESTNSNAYTQR

SEQ ID NO: 479 - protein, 16 aa, Homo sapiens
  MOD_RES (9)..(9): Phosphorylated-Tyr
VPNSNPPEYEFFWGLR

SEQ ID NO: 480 - protein, 14 aa, Homo sapiens
  MOD_RES (1)..(1): Phosphorylated-Tyr
YANNSNYKNDVMIR

SEQ ID NO: 481 - protein, 10 aa, Homo sapiens
  MOD_RES (4)..(4): Phosphorylated-Tyr
KYDYDSSSVR

SEQ ID NO: 482 - protein, 15 aa, Homo sapiens
  MOD_RES (5)..(5): Phosphorylated-Tyr
GQSIYSTSFPQPAPR

SEQ ID NO: 483 - protein, 12 aa, Homo sapiens
  MOD_RES (11)..(11): Phosphorylated-Tyr
LTPTHAASPVYR

SEQ ID NO: 484 - protein, 35 aa, Homo sapiens
  MOD_RES (10)..(10): Phosphorylated-Tyr
SGISTNHADYSSSPAGSPGAQVSLYNSPSVASPAR

SEQ ID NO: 485 - protein, 35 aa, Homo sapiens; identical to SEQ ID NO: 484
  MOD_RES (25)..(25): Phosphorylated-Tyr

SEQ ID NO: 486 - protein, 21 aa, Homo sapiens
  MOD_RES (9)..(9): Phosphorylated-Tyr
VLLPDLEFYVNLGDWPLEHRK

SEQ ID NO: 487 - protein, 12 aa, Homo sapiens
  MOD_RES (9)..(9): Phosphorylated-Tyr
LIHTGEKPYKCL

SEQ ID NO: 488 - protein, 19 aa, Homo sapiens
  MOD_RES (17)..(17): Phosphorylated-Tyr
DKMVLECLLNLMQRDPYWK

SEQ ID NO: 489 - protein, 9 aa, Homo sapiens
  MOD_RES (2)..(2): Phosphorylated-Tyr
VYQETSEMR

SEQ ID NO: 490 - protein, 20 aa, Homo sapiens
  MOD_RES (3)..(3): Phosphorylated-Tyr
HAYHPACLGPSYPTRATRKR

SEQ ID NO: 491 - protein, 20 aa, Homo sapiens
  MOD_RES (12)..(12): Phosphorylated-Tyr
HAYHPACLGPSYPTRATRKR

SEQ ID NO: 492 - protein, 12 aa, Homo sapiens
  MOD_RES (8)..(8): Phosphorylated-Tyr
QSNNVLVYDLLK

SEQ ID NO: 493 - protein, 20 aa, Homo sapiens
  MOD_RES (18)..(18): Phosphorylated-Tyr
NASLASSNNDLQVAEEQYQR

SEQ ID NO: 494 - protein, 14 aa, Homo sapiens
  MOD_RES (3)..(3): Phosphorylated-Tyr
QNYDEMSPAGQISK

SEQ ID NO: 495 - protein, 20 aa, Homo sapiens
  MOD_RES (6)..(6): Phosphorylated-Tyr
DMSGHYQNALYLGDVSERVR

SEQ ID NO: 496 - protein, 27 aa, Homo sapiens
  MOD_RES (17)..(17): Phosphorylated-Tyr
NVPEIAVYPAFEAPPQYVLPTYEMAVK

SEQ ID NO: 497 - protein, 14 aa, Homo sapiens
  MOD_RES (11)..(11): Phosphorylated-Tyr
LEPQIASASEYAHR

(19) United States
(12) Patent Application Publication
(10) Pub. No.: US 2010/0009463 A1
(43) Pub. Date: Jan. 14, 2010

(54) Reagents for the detection of protein phosphorylation in signaling pathways

(76) Inventors: Peter Hornbeck, Magnolia, MA (US); Valerie Goss, Seabrook, NH (US); Kimberly Lee, Seattle, WA (US); Ting-Lei Gu, Woburn, MA (US); Albrecht Moritz, Salem, MA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(21) Appl. No.: 12/309,313

(22) PCT Filed: Jul. 13, 2007
(86) PCT No.: PCT/US07/73542

§ 371 Date: Jan. 13, 2009

Related U.S. Application Data
(60) Provisional application No. 60/830,548, filed on Jul. 13, 2006.

(51) Int. Cl. G01N 33/566; C07K 16/18; C07K 14/47; C12N 5/16; C12N 5/18

(52) U.S. Cl. 436/501; 530/387.9; 530/350; 435/331

(57) ABSTRACT

The invention discloses novel phosphorylation sites identified in signal transduction proteins and pathways, and provides phosphorylation-site specific antibodies and heavy-isotope labeled peptides (AQUA peptides) for the selective detection and quantification of these phosphorylated sites/proteins, as well as methods of using the reagents for such purpose. Among the phosphorylation sites identified are sites occurring in the following protein types: adaptor/scaffold proteins, adhesion/extracellular matrix protein, apoptosis proteins, calcium binding proteins, cell cycle regulation proteins, chaperone proteins, chromatin, DNA binding/repair/replication proteins, cytoskeletal proteins, endoplasmic reticulum or golgi proteins, enzyme proteins, G/regulator proteins, inhibitor proteins, motor/contractile proteins, phosphatase, protease, Ser/Thr protein kinases, protein kinase (Tyr)s, receptor/channel/cell surface proteins, RNA binding proteins, transcriptional regulators, tumor suppressor proteins, ubiquitan conjugating system proteins and proteins of unknown function.

### RELATED APPLICATIONS

Pursuant to 35 U.S.C. §119(e) this application claims the benefit of, and priority to, provisional application U.S. Ser. No. 60/830,548, filed Jul. 13, 2006, the disclosure of which is incorporated herein, in its entirety, by reference.

### TECHNICAL FIELD

The invention relates generally to a variety of moieties and tools for the detection of protein phosphorylation. Moreover, the invention relates to the use of the same for diagnostic and therapeutic purposes.

### BACKGROUND

The activation of proteins by post-translational modification is an important cellular mechanism for regulating most aspects of biological organization and control, including growth, development, homeostasis, and cellular communication. Cellular signal transduction pathways involve protein kinases, protein phosphatases, and phosphoprotein-interacting domain (e.g., SH2, PTB, WW, FHA, 14-3-3) containing cellular proteins to provide multidimensional, dynamic and reversible regulation of many biological activities. See e.g., Sawyer et al., Med Chem. 1(3): 293-319 (2005).

Protein phosphorylation on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 520 different protein kinases, making them the most abundant class of enzymes known. See Hunter, Nature 411: 355-65 (2001). Most kinases phosphorylate many different substrate proteins, at distinct tyrosine, serine, and/or threonine residues. Indeed, it is estimated that one-third of all proteins encoded by the human genome are phosphorylated, and many are phosphorylated at multiple sites by different kinases. See Graves et al., Pharmacol. Ther. 82: 111-21 (1999).

Many of these phosphorylation sites regulate critical biological processes and may prove to be important for diagnostic or therapeutic modalities useful in the treatment and management of many pathological conditions and diseases, including inter alia cancer, developmental disorders, as as inflammatory, immune, metabolic and bone diseases.

For example, of the more than 100 dominant oncogenes identified to date, 46 are protein kinases. See Hunter, supra. Understanding which proteins are modified by these kinases will greatly expand our understanding of the molecular mechanisms underlying oncogenic transformation. Therefore, the identification of, and ability to detect, phosphorylation sites on a wide variety of cellular proteins is crucially important to understanding the key signaling proteins and pathways implicated in the progression of many disease states.

Understanding reversible protein phosphorylation and its role in the operation and interrelationship between cellular components and functions provides the opportunity to gain a finer appreciation of cellular regulation. In spite of the importance of protein modification, phosphorylation is not yet well understood due to the extraordinary complexity of signaling pathways, and the slow development of the technology necessary to unravel it.

In many instances, such knowledge is likely to provide valuable tools useful to evaluate, and possibly to manipulate target pathways, ultimately altering the functional status of a given cell for a variety of purposes.

The importance of protein kinase-regulated signal transduction pathways is underscored by a number of drugs designed to treat various cancer types by the inhibition of target protein kinases at the apex or intermediary levels of pathways implicated in cancer development. See Stern et al., Expert Opin. Ther. Targets 9(4):851-60 (2005).

Leukemia, a disease in which a number of underlying signal transduction events have been elucidated, has become a disease model for phosphoproteomic research and development efforts. As such, it represent a paradigm leading the way for many other programs seeking to address many classes of diseases (See, Harrison's Principles of Internal Medicine, McGraw-Hill, New York, N.Y.)

Depending on the cell type involved and the rate by which the disease progresses leukemia can be defined as acute or chronic myelogenous leukemia (AML or CML), or acute and chronic lymphocytic leukemia (ALL or CLL).

Most varieties of leukemia are generally characterized by genetic alterations e.g., chromosomal translocations, deletions or point mutations resulting in the constitutive activation of protein kinase genes, and their products, particularly tyrosine kinases. The most well known alteration is the oncogenic role of the chimeric BCR-Abl gene. See Nowell, Science 132: 1497 (1960)). The resulting BCR-Abl kinase protein is constitutively active and elicits characteristic signaling pathways that have been shown to drive the proliferation and survival of CML cells (see Daley, Science 247: 824-830 (1990); Raitano et al., Biochim. Biophys. Acta. December 9; 1333(3): F201-16 (1997)).

The recent success of Imanitib (also known as ST1571 or Gleevec®), the first molecularly targeted compound designed to specifically inhibit the tyrosine kinase activity of BCR-Abl, provided critical confirmation of the central role of BCR-Abl signaling in the progression of CML (see Schindler et al., Science 289: 1938-1942 (2000); Nardi et al., Curr. Opin. Hematol. 11: 35-43 (2003)).

The success of Gleevec® now serves as a paradigm for the development of targeted drugs designed to block the activity of other tyrosine kinases known to be involved in many diseased including leukemias and other malignancies (see, e.g., Sawyers, Curr. Opin. Genet. Dev. February; 12(1): 111-5 (2002); Druker, Adv. Cancer Res. 91:1-30 (2004)). For example, recent studies have demonstrated that mutations in the FLT3 gene occur in one third of adult patients with AML. FLT3 (Fms-like tyrosine kinase 3) is a member of the class III receptor tyrosine kinase (RTK) family including FMS, platelet-derived growth factor receptor (PDGFR) and c-KIT (see Rosnet et al., Crit. Rev. Oncog. 4: 595-613 (1993). In 20-27% of patients with AML, an internal tandem duplication in the juxta-membrane region of FLT3 can be detected (see Yokota et al., Leukemia 11: 1605-1609 (1997)). Another 7% of patients have mutations within the active loop of the second kinase domain, predominantly substitutions of aspartate residue 835 (D835), while additional mutations have been described (see Yamamoto et al., Blood 97: 2434-2439 (2001); Abu-Duhier et al., Br. J. Haematol. 113: 983-988 (2001)). Expression of mutated FLT3 receptors results in constitutive tyrosine phosphorylation of FLT3, and subsequent phosphorylation and activation of downstream molecules such as STAT5, Akt and MAPK, resulting in factor-independent growth of hematopoietic cell lines.

Altogether, FLT3 is the single most common activated gene in AML known to date. This evidence has triggered an intensive search for FLT3 inhibitors for clinical use leading to at least four compounds in advanced stages of clinical development, including: PKC412 (by Novartis), CEP-701 (by Cephalon), MLN518 (by Millenium Pharmaceuticals), and SU5614 (by Sugen/Pfizer) (see Stone et al., Blood (in press)(2004); Smith et al., Blood 103: 3669-3676 (2004); Clark et al., Blood 104: 2867-2872 (2004); and Spiekerman et al., Blood 101: 1494-1504 (2003)).

There is also evidence indicating that kinases such as FLT3, c-KIT and Abl are implicated in some cases of ALL (see Cools et al., Cancer Res. 64: 6385-6389 (2004); Hu, Nat. Genet. 36: 453-461 (2004); and Graux et al., Nat. Genet. 36: 1084-1089 (2004)). In contrast, very little is know regarding any causative role of protein kinases in CLL, except for a high correlation between high expression of the tyrosine kinase ZAP70 and the more aggressive form of the disease (see Rassenti et al., N. Eng. J. Med. 351. 893-901 (2004)).

Despite the identification of a few key molecules involved in progression of leukemia, the vast majority of signaling protein changes underlying this disease remains unknown. There is, therefore, relatively scarce information about kinase-driven signaling pathways and phosphorylation sites relevant to the different types of leukemia. This has hampered a complete and accurate understanding of how protein activation within signaling pathways is driving these complex cancers. Accordingly, there is a continuing and pressing need to unravel the molecular mechanisms of kinase-driven oncogenesis in leukemia by identifying the downstream signaling proteins mediating cellular transformation in this disease. Identifying particular phosphorylation sites on such signaling proteins and providing new reagents, such as phospho-specific antibodies and AQUA peptides, to detect and quantify them remains particularly important to advancing our understanding of the biology of this disease.

Presently, diagnosis of leukemia is made by tissue biopsy and detection of different cell surface markers. However, misdiagnosis can occur since some leukemia cases can be negative for certain markers, and because these markers may not indicate which genes or protein kinases may be deregulated. Although the genetic translocations and/or mutations characteristic of a particular form of leukemia can be sometimes detected, it is clear that other downstream effectors of constitutively active kinases having potential diagnostic, predictive, or therapeutic value, remain to be elucidated. Accordingly, identification of downstream signaling molecules and phosphorylation sites involved in different types of leukemia and development of new reagents to detect and quantify these sites and proteins may lead to improved diagnostic/prognostic markers, as well as novel drug targets, for the detection and treatment of this disease.

### SUMMARY OF THE INVENTION

Several novel protein phosphorylation sites have been identified in a variety of cell lines. Such novel phosphorylation sites (tyrosine), and their corresponding parent proteins are reported (see Table 1). The elucidation of these sites at long last provides the elements necessary to attain those much needed proteomics tools and modalities.

The invention discloses novel phosphorylation sites identified in signal transduction proteins and pathways underlying various disease states including for example human leukemias. The invention thus provides new reagents, including phosphorylation-site specific antibodies and AQUA peptides, for the selective detection and quantification of these phosphorylated sites/proteins. Also provided are methods of using the reagents of the invention for the detection and quantification of the disclosed phosphorylation sites.

### BRIEF DESCRIPTION OF THE DRAWINGS

FIG. 1—Is a diagram broadly depicting the immunoaffinity isolation and mass-spectrometric characterization methodology (IAP) employed to identify the novel phosphorylation sites disclosed herein.

FIG. 2—Is a table (corresponding to Table 1) enumerating the protein phosphorylation sites disclosed herein: Column A=the name of the parent protein; Column B=the SwissProt accession number for the protein (human sequence); Column C=the protein type/classification; Column D=the tyrosine residue (in the parent protein amino acid sequence) at which phosphorylation occurs within the phosphorylation site; Column E=the phosphorylation site sequence encompassing the phosphorylatable residue (residue at which phosphorylation occurs (and corresponding to the respective entry in Column D) appears in lowercase; Column F=the type of leukemia in which the phosphorylation site was discovered; and Column G=the cell type(s), tissue(s) and/or patient(s) in which the phosphorylation site was discovered.

FIG. 3—is an exemplary mass spectrograph depicting the detection of the tyrosine 237 phosphorylation site in GRASP (see Row 10 in FIG. 2/Table 1), as further described in Example 1 (red and blue indicate ions detected in MS/MS spectrum); Y* indicates the phosphorylated tyrosine (shown as lowercase “y” in FIG. 2).

FIG. 4—is an exemplary mass spectrograph depicting the detection of the tyrosine 96 phosphorylation site in GOT2 (see Row 112 in FIG. 2/Table 1), as further described in Example 1 (red and blue indicate ions detected in MS/MS spectrum); Y* indicates the phosphorylated tyrosine (shown as lowercase “y” in FIG. 2).

FIG. 5—is an exemplary mass spectrograph depicting the detection of the tyrosine 314 phosphorylation site in GAPDH (see Row 99 in FIG. 2/Table 1), as further described in Example 1 (red and blue indicate ions detected in MS/MS spectrum); Y* indicates the phosphorylated serine (shown as lowercase “y” in FIG. 2).

FIG. 6—is an exemplary mass spectrograph depicting the detection of the tyrosine 84 phosphorylation site in LDH-B (see Row 134 in FIG. 2/Table 1), as further described in Example 1 (red and blue indicate ions detected in MS/MS spectrum); Y* indicates the phosphorylated tyrosine (shown as lowercase “y” in FIG. 2)

FIG. 7—is an exemplary mass spectrograph depicting the detection of the tyrosine 1154 phosphorylation site in HGK (see Row 198 in FIG. 2/Table 1), as further described in Example 1 (red and blue indicate ions detected in MS/MS spectrum); Y* indicates the phosphorylated tyrosine (shown as lowercase “y” in FIG. 2).

FIG. 8—is an exemplary mass spectrograph depicting the detection of the tyrosine 38 phosphorylation site in MCEMP (see Row 259 in FIG. 2/Table 1), as further described in Example 1 (red and blue indicate ions detected in MS/MS spectrum); Y* indicates the phosphorylated tyrosine (shown as lowercase “y” in FIG. 2).

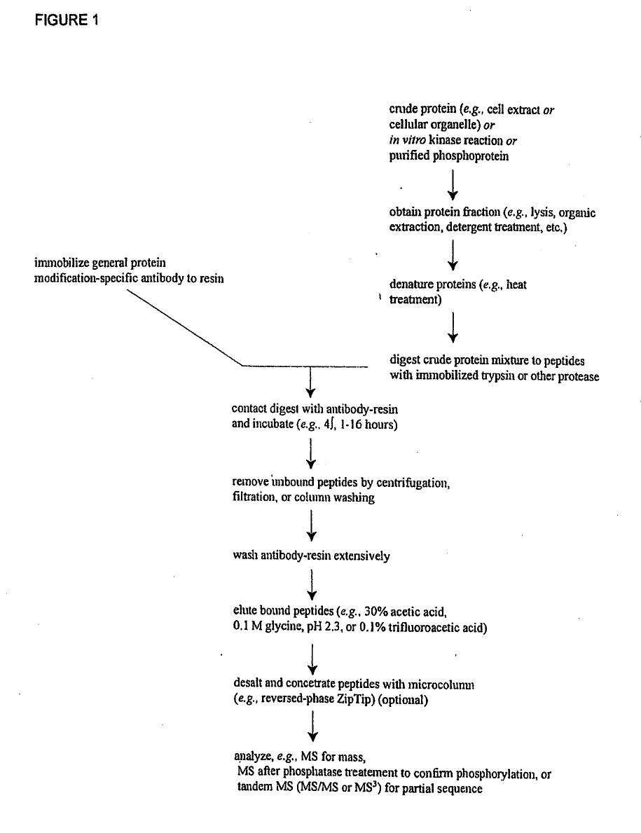

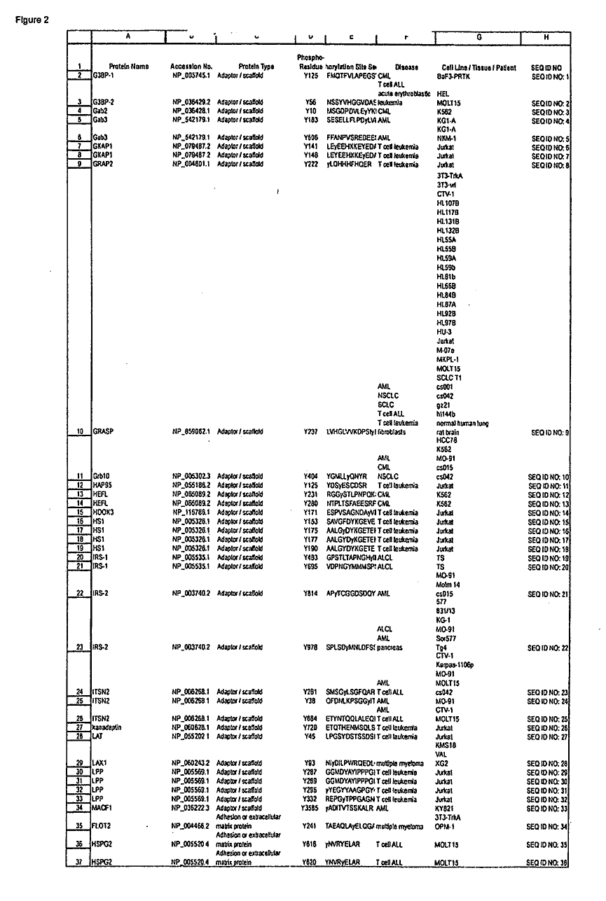

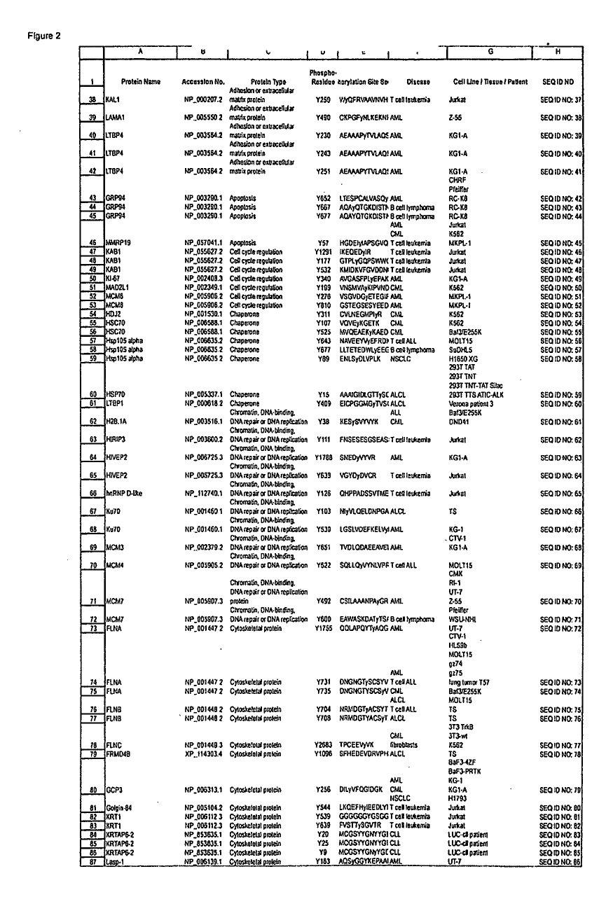

### DETAILED DESCRIPTION

Several novel protein phosphorylation sites have been identified in a variety of cell lines. Such novel phosphorylation sites (tyrosine), and their corresponding parent proteins are reported (see Table 1). The elucidation of these sites at long last provides the elements necessary to attain those much needed proteomics tools and modalities.

The disclosure of the phosphorylation sites provides the key to the production of new moieties, compositions and methods to specifically detect and/or to quantify these phosphorylated sites/proteins. Such moieties include for example reagents, such as phosphorylation site-specific antibodies and AQUA peptides (heavy-isotope labeled peptides). Such reagents are highly useful, inter alia, for studying signal transduction events underlying the progression of many diseases known or suspected to involve protein phosphorylation e.g., leukemia in a mammal. Accordingly, the invention provides novel reagents—phospho-specific antibodies and AQUA peptides—for the specific detection and/or quantification of a target signaling protein/polypeptide (e.g., a signaling protein/polypeptide implicated in leukemia) only when phosphorylated (or only when not phosphorylated) at a particular phosphorylation site disclosed herein. The invention also provides methods of detecting and/or quantifying one or more phosphorylated target signaling protein/polypeptide using the phosphorylation-site specific antibodies and AQUA peptides of the invention.

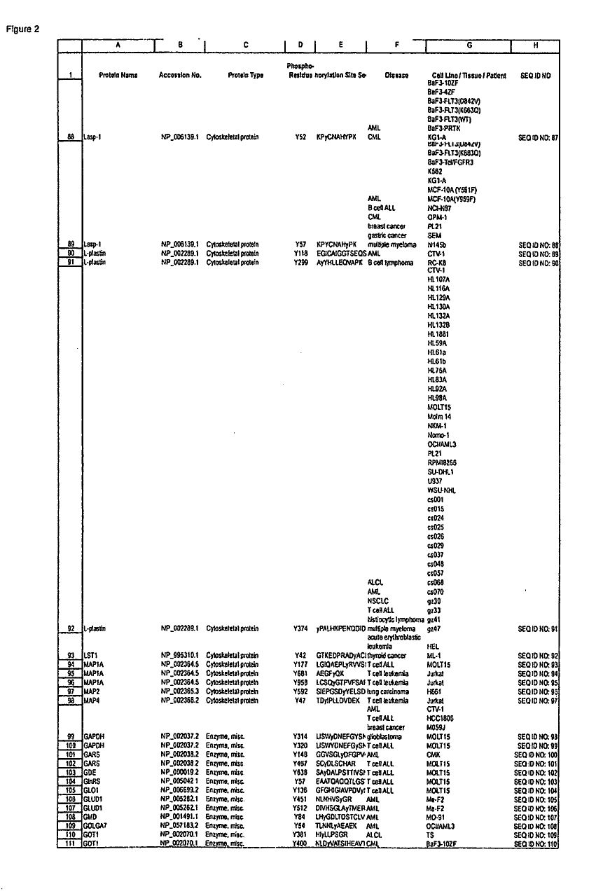

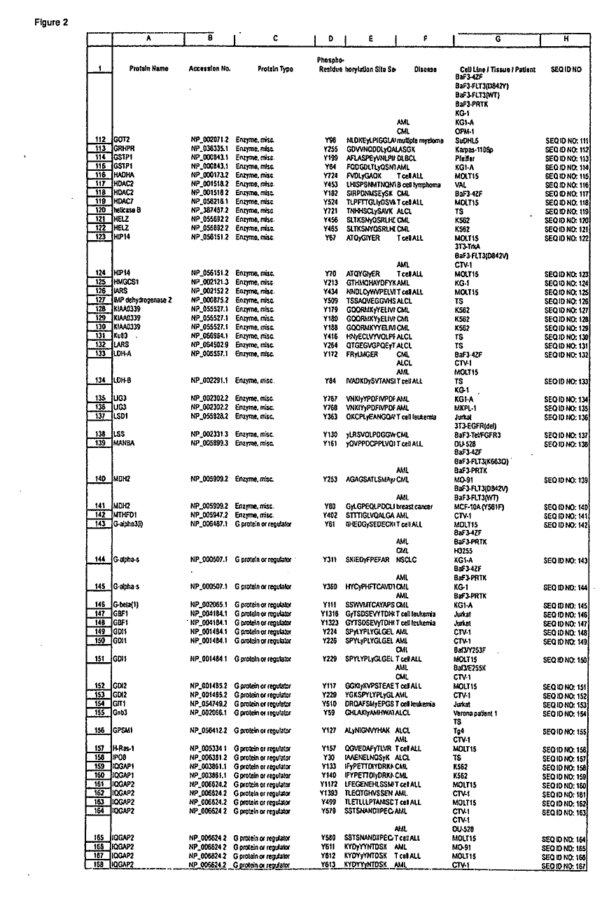

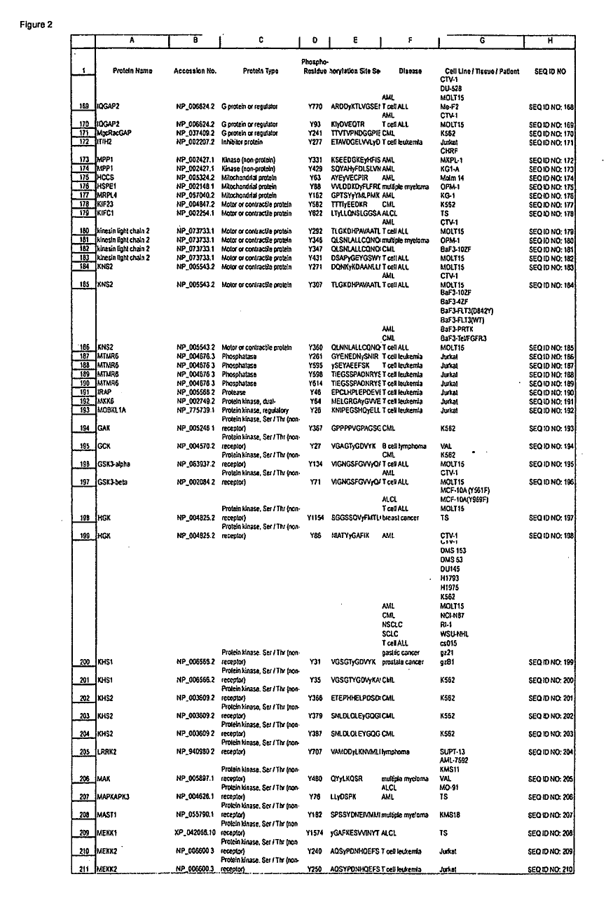

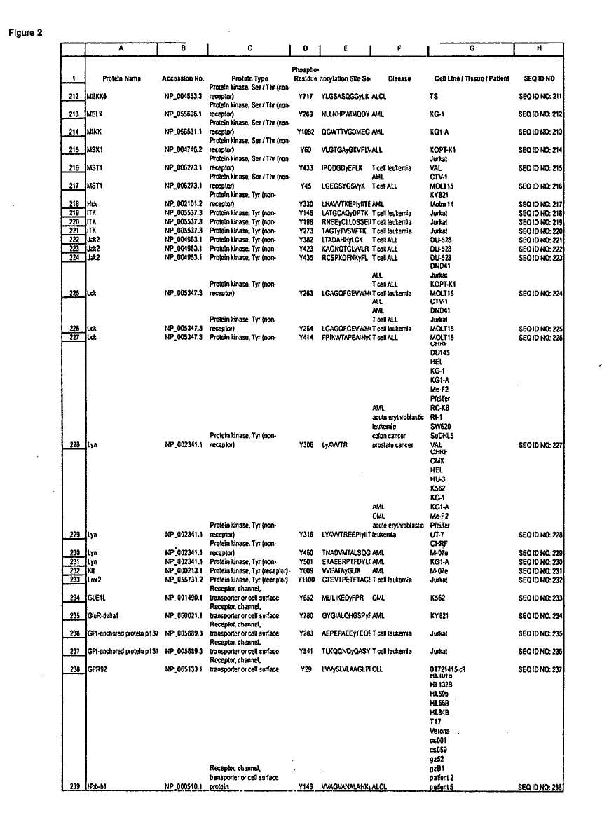

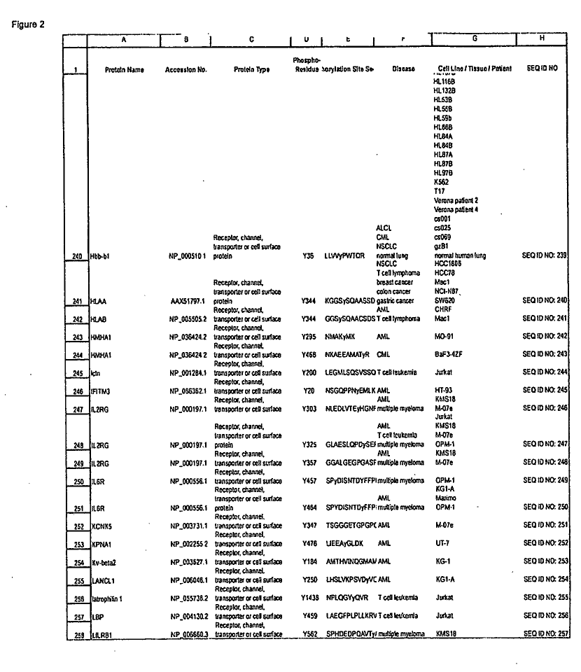

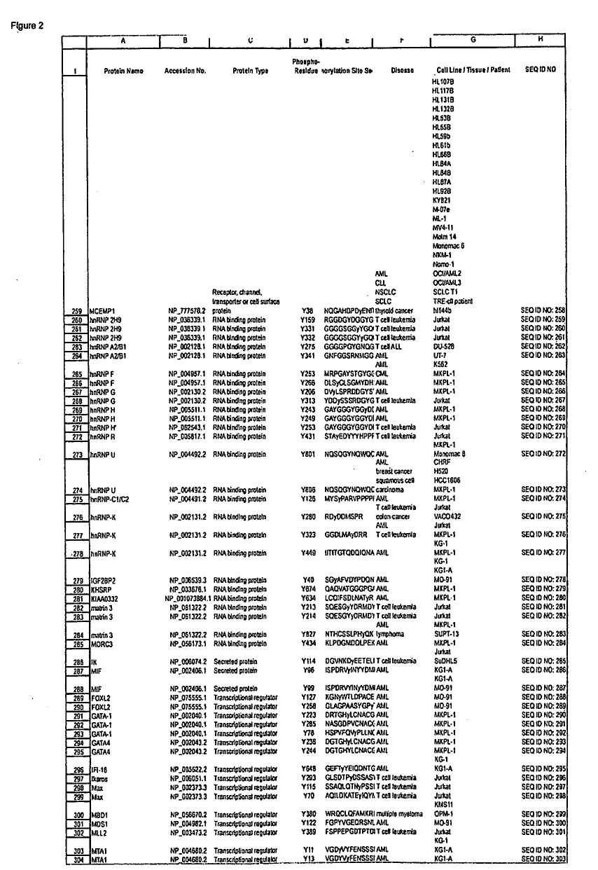

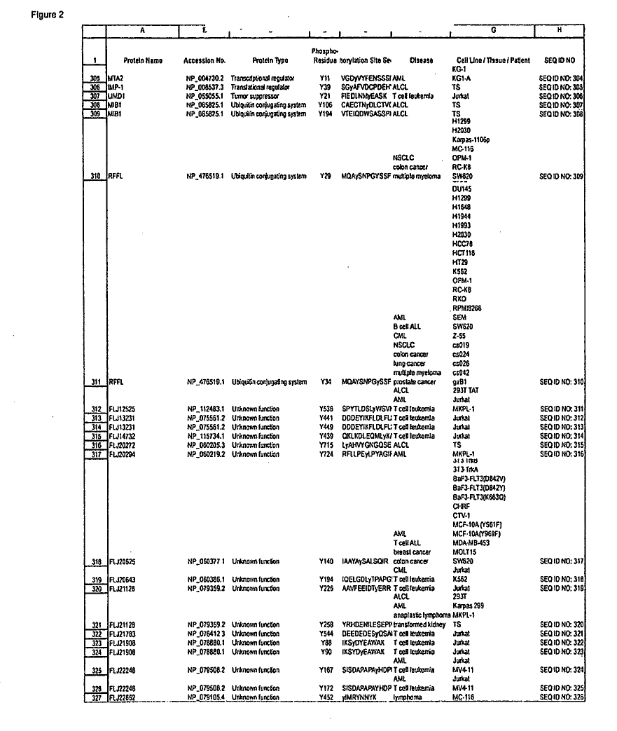

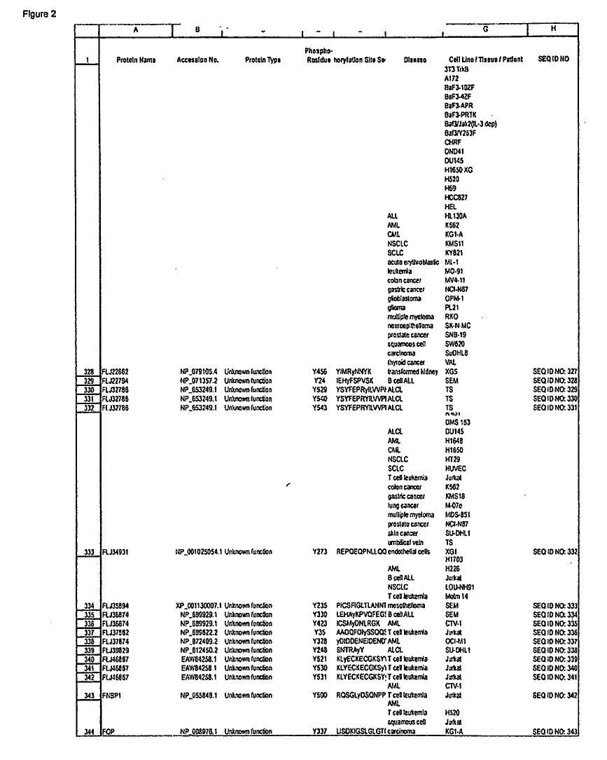

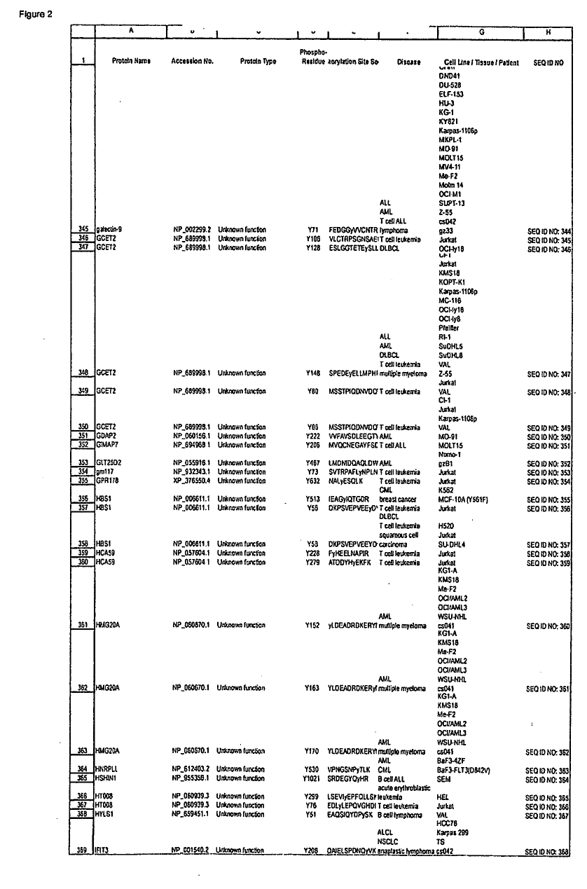

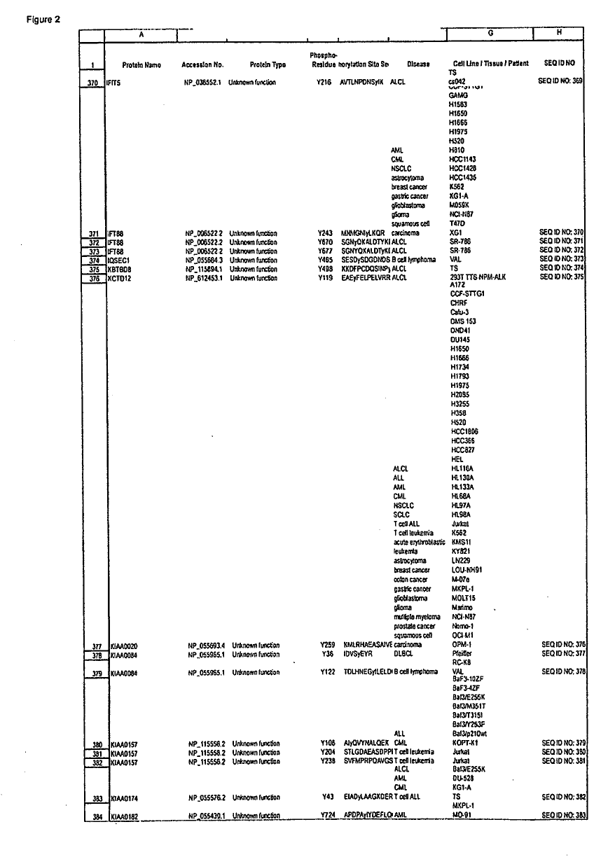

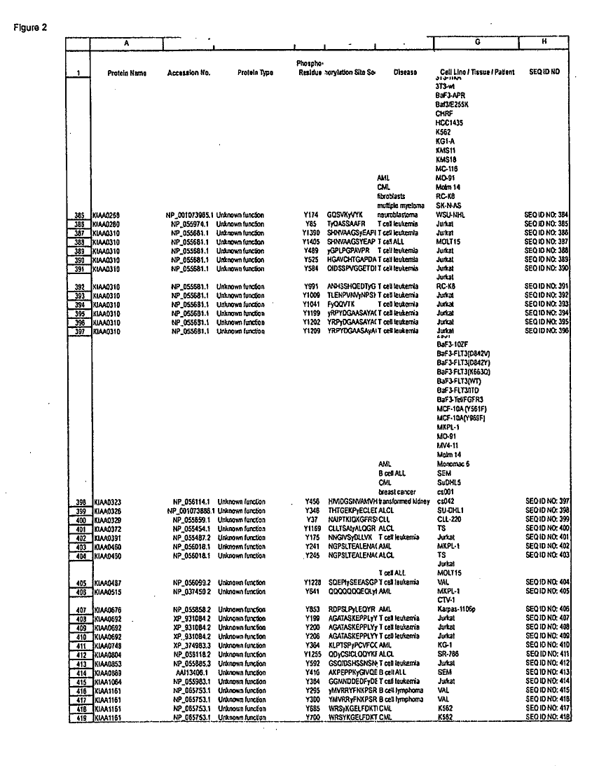

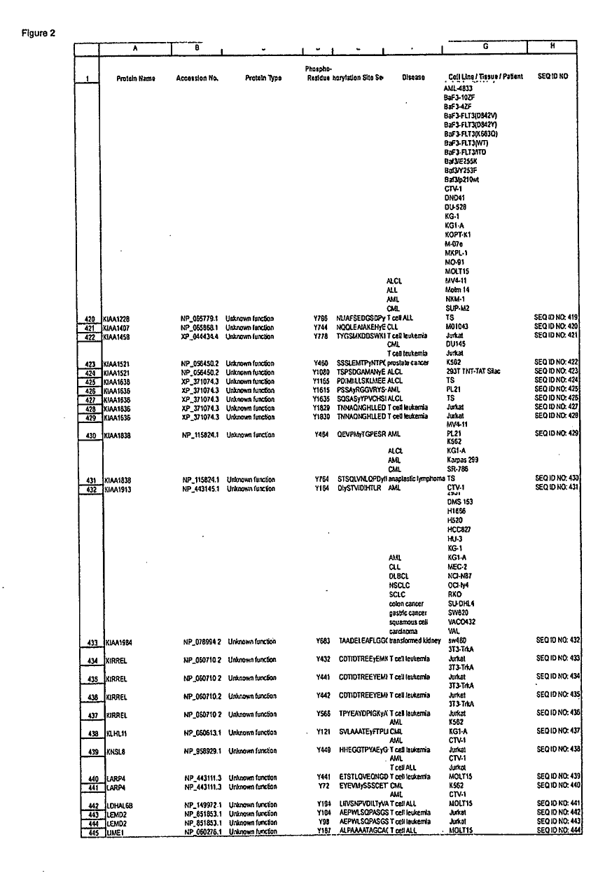

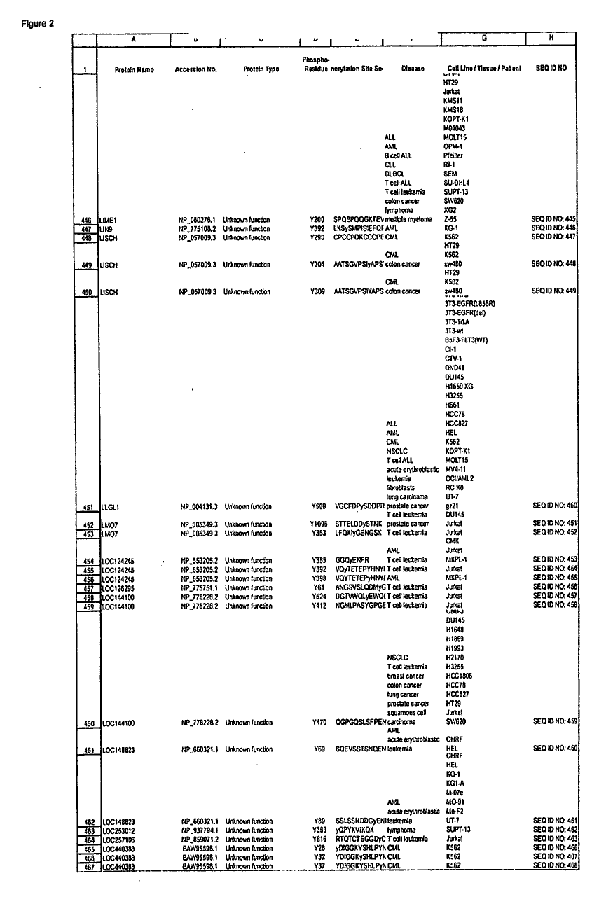

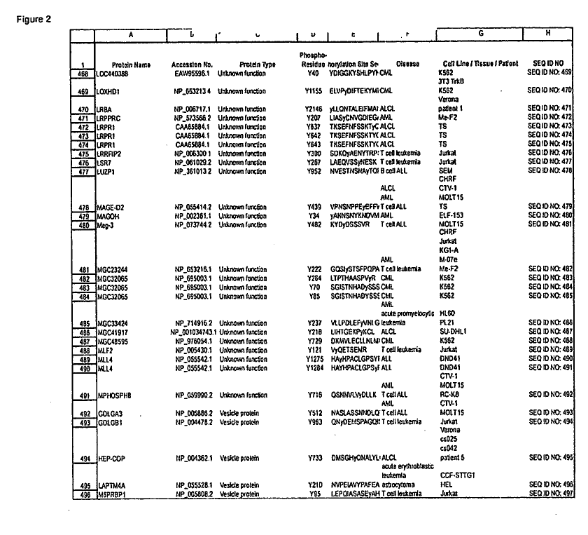

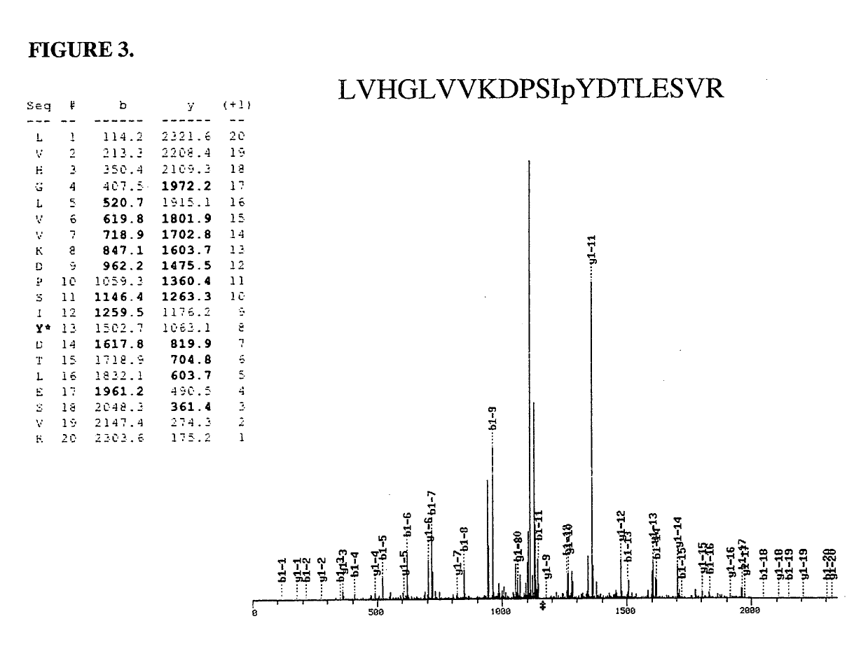

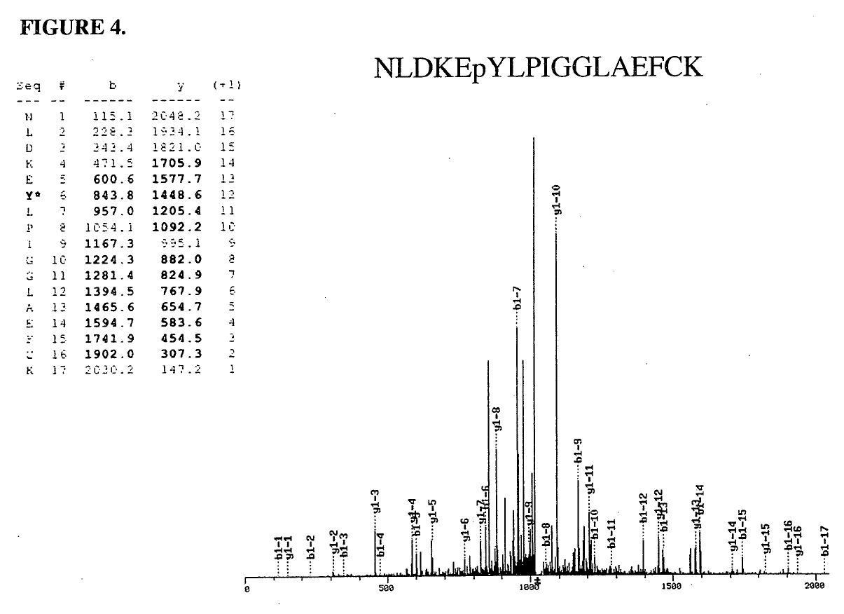

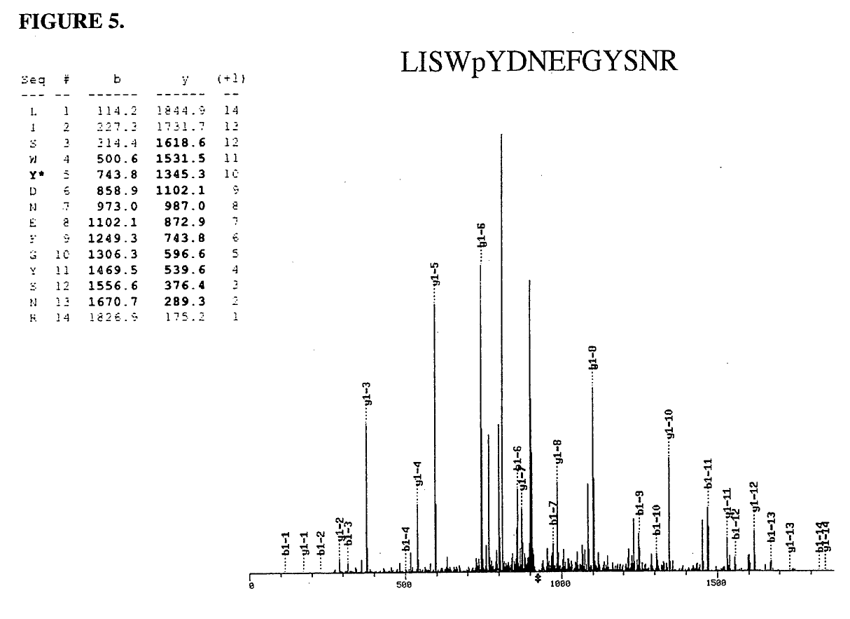

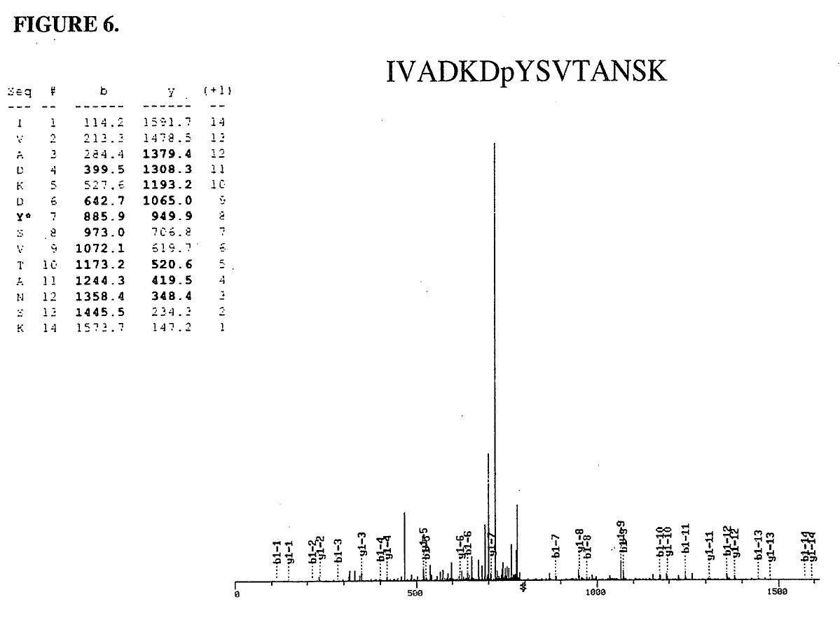

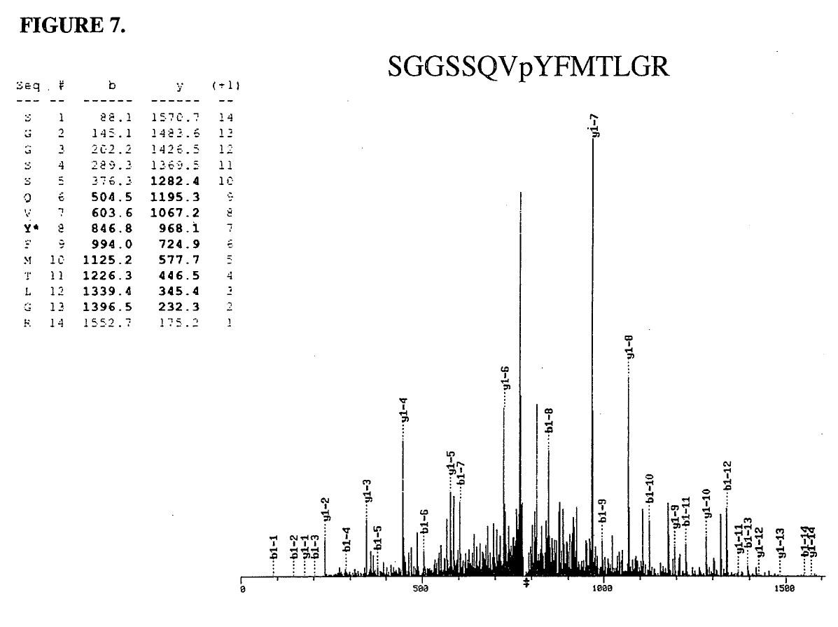

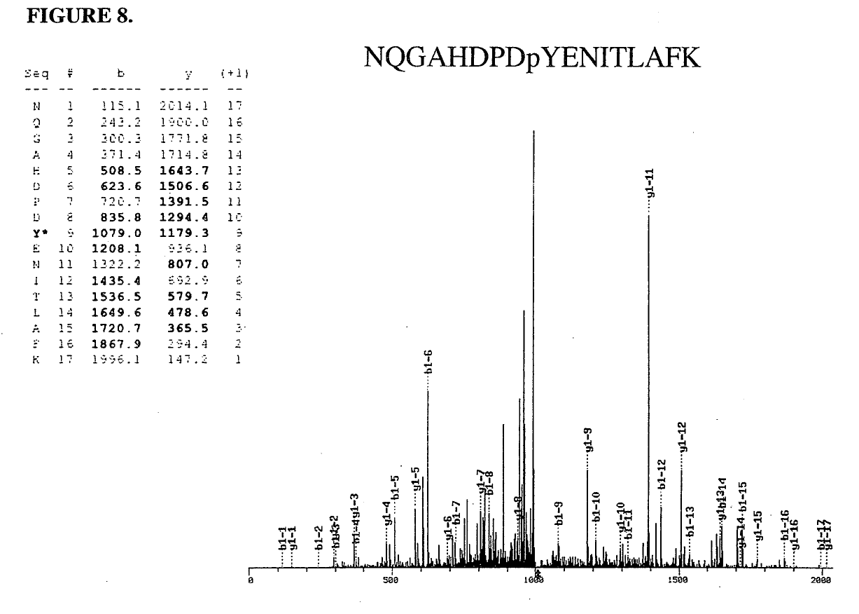

These phosphorylation sites correspond to numerous different parent proteins (the full sequences (human) of which are all publicly available in SwissProt database and their Accession numbers listed in Column B of Table 1/FIG. 2), each of which are have been linked to specific functions in the literature and thus may be organized into discrete protein type groups, for example adaptor/scaffold proteins, cytoskeletal proteins, protein kinases, and DNA binding proteins, etc. (see Column C of Table 1), the phosphorylation of which is relevant to signal transduction activity (e.g., underlying AML, CML, CLL, and ALL), as disclosed herein.

In part, the invention provides an isolated phosphorylation site-specific antibody that specifically binds a given target signaling protein/polypeptide only when phosphorylated (or not phosphorylated, respectively) at a particular tyrosine enumerated in Column D of Table 1/FIG. 2 comprised within the phosphorylatable peptide site sequence enumerated in corresponding Column E. In further part, the invention provides a heavy-isotope labeled peptide (AQUA peptide) for the detection and quantification of a given target signaling protein/polypeptide, the labeled peptide comprising a particular phosphorylatable peptide site/sequence enumerated in Column E of Table 1/FIG. 2 herein. For example, among the reagents provided by the invention is an isolated phosphorylation site-specific antibody that specifically binds the Gab2 adaptor/scaffold protein only when phosphorylated (or only when not phosphorylated) at tyrosine 10 (see Row 4 (and Columns D and E) of Table 1/FIG. 2). By way of further example, among the group of reagents provided by the invention is an AQUA peptide for the quantification of phosphorylated GRP94 apoptosis protein, the AQUA peptide comprising the phosphorylatable peptide sequence listed in Column E, Row 43, of Table 1/FIG. 2 (which encompasses the phosphorylatable tyrosine at position 652).

In one embodiment, the invention provides an isolated phosphorylation site-specific antibody that specifically binds a target signaling protein/polypeptide selected from Column A of Table 1 (Rows 2-464, 467496) only when phosphorylated at the tyrosine residue listed in corresponding Column D of Table 1, comprised within the phosphorylatable peptide sequence listed in corresponding Column E of Table 1 (SEQ ID NOs: 1-463, 466-498), wherein said antibody does not bind said signaling protein when not phosphorylated at said tyrosine. In another embodiment, the invention provides an isolated phosphorylation site-specific antibody that specifically binds a target signaling protein/polypeptide selected from Column A of Table 1 only when not phosphorylated at the tyrosine residue listed in corresponding Column D of Table 1, comprised within the peptide sequence listed in corresponding Column E of Table 1 (SEQ ID NOs: 1-463, 466-498), wherein said antibody does not bind said signaling protein when phosphorylated at said tyrosine. Such reagents enable the specific detection of phosphorylation (or non-phosphorylation) of a novel phosphorylatable site disclosed herein. The invention further provides immortalized cell lines producing such antibodies. In one embodiment, the immortalized cell line is a rabbit or mouse hybridoma.

In another embodiment, the invention provides a heavy-isotope labeled peptide (AQUA peptide) for the quantification of a target signaling protein/polypeptide selected from Column A of Table 1, said labeled peptide comprising the phosphorylatable peptide sequence listed in corresponding Column E of Table 1 (SEQ ID NOs: 1-463, 466-498), which sequence comprises the phosphorylatable tyrosine listed in corresponding Column D of Table 1. In certain embodiments, the phosphorylatable tyrosine within the labeled peptide is phosphorylated, while in other embodiments, the phosphorylatable residue within the labeled peptide is not phosphorylated.

Reagents (antibodies and AQUA peptides) provided by the invention may conveniently be grouped by the type of target signaling protein/polypeptide in which a given phosphorylation site (for which reagents are provided) occurs. The protein types for each respective protein (in which a phosphorylation site has been discovered) are provided in Column C of Table 1/FIG. 2, and include: adaptor/scaffold proteins, adhesion/extracellular matrix protein, apoptosis proteins, calcium binding proteins, cell cycle regulation proteins, chaperone proteins, chromatin, DNA binding/repair/replication proteins, cytoskeletal proteins, endoplasmic reticulum or golgi proteins, enzyme proteins, G/regulator proteins, inhibitor proteins, motor/contractile proteins, phosphatase, protease, Ser/Thr protein kinases, protein kinase (Tyr)s, receptor/channel/cell suface proteins, RNA binding proteins, transcriptional regulators, tumor suppressor proteins, ubiquitan conjugating system proteins and proteins of unknown function. Each of these distinct protein groups is a subset of target signaling protein/polypeptide phosphorylation sites disclosed herein, and reagents for their detection/quantification may be considered a subset of reagents provided by the invention.

Subsets of the phosphorylation sites (and their corresponding proteins) disclosed herein are those occurring on the following protein types/groups listed in Column C of Table 1/FIG. 2 adaptor/scaffold proteins, calcium binding proteins, chromatin or DNA binding/repair/replication proteins, cytoskeletal proteins, enzyme proteins, protein kinases (Tyr), protein kinases (Ser/Thr), receptor/channel/transporter/cell suface proteins, transcriptional regulators and translational regulators. Accordingly, among subsets of reagents provided by the invention are isolated antibodies and AQUA peptides useful for the detection and/or quantification of the foregoing protein/phosphorylation site subsets.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

In one subset of embodiments, there is provided:
(i) An isolated phosphorylation site-specific antibody that specifically binds an adaptor/scaffold protein selected from Column A, Rows 2-34, of Table 1 only when phosphorylated at the tyrosine listed in corresponding Column D, Rows 2-34, of Table 1, comprised within the phosphorylatable peptide sequence listed in corresponding Column E, Rows 2-34, of Table 1 (SEQ [D NOs: 1-33), wherein said antibody does not bind said protein when not phosphorylated at said tyrosine.
(ii) An equivalent antibody to (i) above that only binds the adaptor/scaffold protein when not phosphorylated at the disclosed site (and does not bind the protein when it is phosphorylated at the site).
(iii) A heavy-isotope labeled peptide (AQUA peptide) for the quantification of an adaptor/scaffold protein selected from Column A, Rows 2-34, said labeled peptide comprising the phosphorylatable peptide sequence listed in corresponding Column E, Rows 2-34, of Table 1 (SEQ ID NOs: 1-33), which sequence comprises the phosphorylatable tyrosine listed in corresponding Column D, Rows 2-34, of Table 1.

Among this subset of reagents, antibodies and AQUA peptides for the detection/quantification of the following adaptor/scaffold protein phosphorylation sites are: GRASP (Y237), Grb10 (Y404), IRS-1 (Y483), IRS-2 (Y978) and ITSN2 (Y261) (see SEQ ID NOs: 9, 10, 19, 22 and 23).

In a second subset of embodiments there is provided:
(i) An isolated phosphorylation site-specific antibody that specifically binds a cell cycle regulation protein selected from Column A, Rows 47-53, of Table 1 only when phosphorylated at the tyrosine listed in corresponding Column D, Rows 47-53, of Table 1, comprised within the phosphorylatable peptide sequence listed in corresponding Column E, Rows 47-53, of Table 1 (SEQ ID NOs: 46-52), wherein said antibody does not bind said protein when not phosphorylated at said tyrosine.
(ii) An equivalent antibody to (i) above that only binds the cell cycle regulation protein when not phosphorylated at the disclosed site (and does not bind the protein when it is phosphorylated at the site).
(iii) A heavy-isotope labeled peptide (AQUA peptide) for the quantification of a signaling protein that is a cell cycle regulation protein selected from Column A, Rows 47-53, said labeled peptide comprising the phosphorylatable peptide sequence listed in corresponding Column E, Rows 47-53, of Table 1 (SEQ ID NOs: 46-52), which sequence comprises the phosphorylatable tyrosine listed in corresponding Column D, Rows 47-53, of Table 1.

Among this subset of reagents, antibodies and AQUA peptides for the detection/quantification of the following cell cycle regulation protein phosphorylation sites are: K1-67 (Y340) and MAD2L1 (Y199) (see SEQ ID NOs: 49 and 50).

In another subset of embodiments there is provided:
(i) An isolated phosphorylation site-specific antibody that specifically binds a chaperone protein selected from Column A, Rows 54-61, of Table 1 only when phosphorylated at the tyrosine listed in corresponding Column D, Rows 54-61, of Table 1, comprised within the phosphorylatable peptide sequence listed in corresponding Column E, Rows 54-61, of Table 1 (SEQ ID NOs: 53-60), wherein said antibody does not bind said protein when not phosphorylated at said tyrosine.
(ii) An equivalent antibody to (i) above that only binds the chaperone protein when not phosphorylated at the disclosed site (and does not bind the protein when it is phosphorylated at the site).
(iii) A heavy-isotope labeled peptide (AQUA peptide) for the quantification of a signaling protein that is a chaperone protein selected from Column A, Rows 54-61, said labeled peptide comprising the phosphorylatable peptide sequence listed in corresponding Column E, Rows 54-61, of Table 1 (SEQ ID NOs: 53-60), which sequence comprises the phosphorylatable tyrosine listed in corresponding Column D, Rows 54-61, of Table 1.

Among this subset of reagents, antibodies and AQUA peptides for the detection/quantification of the following chaperone protein phosphorylation sites are: HSC70 (Y107) and HSP70 (Y15) (see SEQ ID NO's: 54 and 59).

In still another subset of embodiments there is provided:
(i) An isolated phosphorylation site-specific antibody that specifically binds a chromatin or DNA binding/repair/replication protein selected from Column A, Rows 62-72, of Table 1 only when phosphorylated at the tyrosine listed in corresponding Column D, Rows 62-72, of Table 1, comprised within the phosphorylatable peptide sequence listed in corresponding Column E, Rows 62-72, of Table 1 (SEQ ID NOs: 61-71), wherein said antibody does not bind said protein when not phosphorylated at said tyrosine.
(ii) An equivalent antibody to (i) above that only binds the chromatin or DNA binding/repair/replication protein when not phosphorylated at the disclosed site (and does not bind the protein when it is phosphorylated at the site).
(iii) A heavy-isotope labeled peptide (AQUA peptide) for the quantification of a signaling protein that is a chromatin or DNA binding/repair/replication protein selected from Column A, Rows 62-72, said labeled peptide comprising the phosphorylatable peptide sequence listed in corresponding Column E, Rows 62-72, of Table 1 (SEQ ID NOs: 61-71), which sequence comprises the phosphorylatable tyrosine listed in corresponding Column D, Rows 62-72, of Table 1.

Among this subset of reagents, antibodies and AQUA peptides for the detection/quantification of the following chromatin or DNA binding/repair/replication protein phosphorylation sites are: Ku70 (Y103), Ku70 (Y530) and MCM7(Y492) (see SEQ ID NOs: 66, 67 and 70).

In still another subset of embodiments there is provided:
(i) An isolated phosphorylation site-specific antibody that specifically binds a cytoskeletal protein selected from Column A, Rows 73-98, of Table 1 only when phosphorylated at the tyrosine listed in corresponding Column D, Rows 73-98, of Table 1, comprised within the phosphorylatable peptide sequence listed in corresponding Column E, Rows 73-98, of Table 1 (SEQ ID NOs: 72-97), wherein said antibody does not bind said protein when not phosphorylated at said tyrosine.
(ii) An equivalent antibody to (i) above that only binds the cytoskeletal protein when not phosphorylated at the disclosed site (and does not bind the protein when it is phosphorylated at the site).
(iii) A heavy-isotope labeled peptide (AQUA peptide) for the quantification of a signaling protein that is a cytoskeletal protein selected from Column A, Rows 73-98, said labeled peptide comprising the phosphorylatable peptide sequence listed in corresponding Column E, Rows 73-98, of Table 1 (SEQ ID NOs: 72-97), which sequence comprises the phosphorylatable tyrosine listed in corresponding Column D, Rows 73-98, of Table 1.

Among this subset of reagents, antibodies and AQUA peptides for the detection/quantification of the following cytoskeletal protein phosphorylation sites are: FLNA (Y735), GCP3 (Y256), LASP-1 (Y57) and L-plastin (Y734) (see SEQ ID NOs: 74, 79, 88 and 91).

In still another subset of embodiments there is provided:
(i) An isolated phosphorylation site-specific antibody that specifically binds an enzyme protein selected from Column A, Rows 99-142, of Table 1 only when phosphorylated at the tyrosine listed in corresponding Column D, Rows 99-142, of Table 1, comprised within the phosphorylatable peptide sequence listed in corresponding Column E, Rows 99-142 of Table 1 (SEQ ID NOs: 98-141), wherein said antibody does not bind said protein when not phosphorylated at said tyrosine.
(ii) An equivalent antibody to (i) above that only binds an enzyme protein when not phosphorylated at the disclosed site (and does not bind the protein when it is phosphorylated at the site).
(iii) A heavy-isotope labeled peptide (AQUA peptide) for the quantification of a signaling protein that is an enzyme protein selected from Column A, Rows 99-142, said labeled peptide comprising the phosphorylatable peptide sequence listed in corresponding Column E, Rows 99-142, of Table 1 (SEQ ID NOs: 98-141), which sequence comprises the phosphorylatable tyrosine listed in corresponding Column D, Rows 99-142, of Table 1.

Among this subset of reagents, antibodies and AQUA peptides for the detection/quantification of the following enzyme protein phosphorylation sites are: GAPDH (Y314), HDAC (Y458), HDAC (Y182), HIP14 (Y70), Ku80 (Y416) and LDH-B (Y84) (see SEQ ID NOs: 98, 116, 117, 123, 130 and 133).

In yet another subset of embodiments, there is provided:
(i) An isolated phosphorylation site-specific antibody that specifically binds a G protein or regulator protein selected from Column A, Rows 143-171, of Table 1 only when phosphorylated at the tyrosine listed in corresponding Column D, Rows 143-171, of Table 1, comprised within the phosphorylatable peptide sequence listed in corresponding Column E, Rows 143-171, of Table 1 (SEQ ID NOs: 142-170), wherein said antibody does not bind said protein when not phosphorylated at said tyrosine.
(ii) An equivalent antibody to (i) above that only binds the G protein or regulator protein when not phosphorylated at the disclosed site (and does not bind the protein when it is phosphorylated at the site).
(iii) A heavy-isotope labeled peptide (AQUA peptide) for the quantification of a signaling protein that is a G protein or regulator protein selected from Column A, Rows 143-171, said labeled peptide comprising the phosphorylatable peptide sequence listed in corresponding Column E, Rows 143-171, of Table 1 (SEQ ID NOs: 142-170), which sequence comprises the phosphorylatable tyrosine listed in corresponding Column D, Rows 143-171, of Table 1.

Among this subset of reagents, antibodies and AQUA peptides for the detection/quantification of the following G protein or regulator protein phosphorylation sites are: G-alpha-s (Y311), Gnb3 (Y59), H-Ras-1 (Y 157) and IQGAP2 (Y770) (see SEQ ID NOs: 143, 154, 156 and 168).

In yet another subset of embodiments, there is provided:
(i) An isolated phosphorylation site-specific antibody that specifically binds a protein kinase (Ser/Thr) selected from Column A, Rows 194-217, of Table 1 only when phosphorylated at the tyrosine listed in corresponding Column D, Rows 194-217, of Table 1, comprised within the phosphorylatable peptide sequence listed in corresponding Column E, Rows 194-217, of Table 1 (SEQ ID NOs: 193-216), wherein said antibody does not bind said protein when not phosphorylated at said tyrosine.
(ii) An equivalent antibody to (i) above that only binds the protein kinase (Ser/Thr) when not phosphorylated at the disclosed site (and does not bind the protein when it is phosphorylated at the site).
(iii) A heavy-isotope labeled peptide (AQUA peptide) for the quantification of a signaling protein that is a protein kinase (Ser/Thr) selected from Column A, Rows 194-217, said labeled peptide comprising the phosphorylatable peptide sequence listed in corresponding Column E, Rows 194-217, of Table 1 (SEQ ID NOs: 193-216), which sequence comprises the phosphorylatable tyrosine listed in corresponding Column D, Rows 194-217, of Table 1.

Among this subset of reagents, antibodies and AQUA peptides for the detection/quantification of the following protein kinase (Ser/Thr) phosphorylation sites are: GSK3-beta (Y71), HGK (Y1154) and KHS1 (Y31) (see SEQ ID NOs: 196, 197 and 199).

In yet another subset of embodiments, there is provided:
(i) An isolated phosphorylation site-specific antibody that specifically binds a protein kinase (Tyr) selected from Column A, Rows 218-233, of Table 1 only when phosphorylated at the tyrosine listed in corresponding Column D, Rows 218-233, of Table 1, comprised within the phosphorylatable peptide sequence listed in corresponding Column E, Rows 218-233, of Table 1 (SEQ ID NOs: 217-232), wherein said antibody does not bind said protein when not phosphorylated at said tyrosine.
(ii) An equivalent antibody to (i) above that only binds the protein kinase (Tyr) when not phosphorylated at the disclosed site (and does not bind the protein when it is phosphorylated at the site).
(iii) A heavy-isotope labeled peptide (AQUA peptide) for the quantification of a signaling protein that is a protein kinase (Tyr) selected from Column A, Rows 218-233, said labeled peptide comprising the phosphorylatable peptide sequence listed in corresponding Column E, Rows 218-233, of Table 1 (SEQ ID NOs: 217-232), which sequence comprises the phosphorylatable tyrosine listed in corresponding Column D, Rows 218-233, of Table 1.

Among this subset of reagents, antibodies and AQUA peptides for the detection/quantification of the following protein kinase (Tyr) phosphorylation sites are: Hck (Y330), Jak2 (Y423), Lck (Y414), Lyn (Y306) and Kit (Y609) (see SEQ ID NOs: 217, 222, 226, 227 and 231).

In still another subset of embodiments, there is provided:
(i) An isolated phosphorylation site-specific antibody that specifically binds a receptor/channel/transporter/cell surface protein selected from Column A, Rows 234-259, of Table 1 only when phosphorylated at the tyrosine listed in corresponding Column D, Rows 234-259, of Table 1, comprised within the phosphorylatable peptide sequence listed in corresponding Column E, Rows 234-259, of Table 1 (SEQ ID NOs: 233-258), wherein said antibody does not bind said protein when not phosphorylated at said tyrosine.
(ii) An equivalent antibody to (i) above that only binds the receptor/channel/transporter/cell surface protein when not phosphorylated at the disclosed site (and does not bind the protein when it is phosphorylated at the site).
(iii) A heavy-isotope labeled peptide (AQUA peptide) for the quantification of a signaling protein that is a receptor/channel/transporter/cell surface protein selected from Column A, Rows 234-259, said labeled peptide comprising the phosphorylatable peptide sequence listed in corresponding Column E, Rows 234-259, of Table 1 (SEQ ID NOs: 233-258), which sequence comprises the phosphorylatable tyrosine listed in corresponding Column D, Rows 234-259, of Table 1.

Among this subset of reagents, antibodies and AQUA peptides for the detection/quantification of the following a receptor/channel/transporter/cell surface protein phosphorylation sites are: IL2RG (Y325) and IL6R (Y464) (see SEQ ID NOs: 247 and 250).

In yet a further subset of embodiments, there is provided:
(i) An isolated phosphorylation site-specific antibody that specifically binds a protein selected from the group consisting of GATA-1 (Y223), GCET2 (Y347), LIME1 (Y200), LLGL1 (Y509), MAGE-D2 (Y439) and HEP-COP (Y733) (Column A, Rows 291, 348, 446, 451, 478 and 494 of Table 1) only when phosphorylated at the tyrosine listed in corresponding Column D of Table 1), said tyrosine comprised within the phosphorylatable peptide sequence listed in corresponding Column E of Table 1 (SEQ ID NOs: 290, 347, 445, 450, 479 and 495), wherein said antibody does not bind said protein when not phosphorylated at said tyrosine.
(ii) An equivalent antibody to (i) above that only binds a protein selected from the group consisting of GATA-1 (Y223), GCET2 (Y347), LIME1 (Y200), LLGL1 (Y509), MAGE-D2 (Y439) and HEP-COP (Y733) (Column A, Rows 291, 348, 446, 451, 478 and 494 of Table 1) when not phosphorylated at the disclosed site (and does not bind the protein when it is phosphorylated at the site).
(iii) A heavy-isotope labeled peptide (AQUA peptide) for the quantification of a protein selected from the group consisting of GATA-1 (Y223), GCET2 (Y347), LIME1 (Y200), LLGL1 (Y509), MAGE-D2 (Y439) and HEP-COP (Y733) (Column A, Rows 291, 348, 446, 451, 478 and 494 of Table 1), said labeled peptide comprising the phosphorylatable peptide sequence listed in corresponding Column E of Table 1 (SEQ ID NOs: 290, 347, 445, 450, 479 and 495), which sequence comprises the phosphorylatable tyrosine listed in corresponding Column D, Rows 291, 348, 446, 451, 478 and 494 of Table 1.

The invention also provides an immortalized cell line producing an antibody of the invention, for example, a cell line producing an antibody within any of the foregoing subsets of antibodies. In an embodiment, the immortalized cell line is a rabbit hybridoma or a mouse hybridoma.

In other embodiments, a heavy-isotope labeled peptide (AQUA peptide) of the invention (for example, an AQUA peptide within any of the foregoing subsets of AQUA peptides) comprises a disclosed site sequence wherein the phosphorylatable tyrosine is phosphorylated. In yet other embodiments, a heavy-isotope labeled peptide of the invention comprises a disclosed site sequence wherein the phosphorylatable tyrosine is not phosphorylated.

The foregoing subsets of reagents of the invention should not be construed as limiting the scope of the invention, which, as noted above, includes reagents for the detection and/or quantification of disclosed phosphorylation sites on any of the other protein type/group subsets (each a subset) listed in Column C of Table 1/FIG. 2.

Also provided by the invention are methods for detecting or quantifying a target signaling protein/polypeptide that is tyrosine phosphorylated, said method comprising the step of utilizing one or more of the above-described reagents of the invention to detect or quantify one or more target Signaling Protein(s)/Polypeptide(s) selected from Column A of Table 1 only when phosphorylated at the tyrosine listed in corresponding Column D of Table 1. In certain embodiments of the methods of the invention, the reagents comprise a subset of reagents as described above. The antibodies according to the invention maybe used in standard (e.g., ELISA or conventional cytometric assays). The invention thus, provides compositions and methods for the detection and/or quantitation of a given target signaling protein or polypeptide in a sample, by contacting the sample and a control sample with one or more antibody of the invention under conditions favoring the binding and thus formation of the complex of the antibody with the protein or peptide. The formation of the complex is then detected according to methods well established and known in the art.

Also provided by the invention is a method for obtaining a phosphorylation profile of a certain protein type or group, for example adaptor/scaffold proteins or cell cycle regulation proteins (Rows 2-34 and Rows 47-53, respectively, of Table 1), that is phosphorylated in a disease signaling pathway, said method comprising the step of utilizing one or more isolated antibody that specifically binds the protein group selected from Column A of Table 1 only when phosphorylated at the tyrosine listed in corresponding Column D, of Table 1, comprised within the phosphorylation site sequence listed in corresponding Column E, to detect the phosphorylation of one or more of said protein group, thereby obtaining a phosphorylation profile for said protein group.

The invention further contemplates compositions, foremost pharmaceutical compositions, containing onr or a more antibody according to the invention formulated together with a pharmaceutically acceptable carrier. One of skill will appreciate that in certain instances the composition of the invention may further comprise other pharmaceutically active moieties. The compounds according to the invention are optionally formulated in a pharmaceutically acceptable vehicle with any of the well-known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well any other pharmacologically active ingredient useful for the treatment of the symptom/condition being treated.

The invention also provides methods of treating a mammal comprising the step of administering such a mammal a therapeutically effective amount of a composition according to the invention. As used herein, by “treating” is meant reducing, preventing, and/or reversing the symptoms in the individual to which a compound of the invention has been administered, as compared to the symptoms of an individual not being treated according to the invention. A practitioner will appreciate that the compounds, compositions, and methods described herein are to be used in concomitance with continuous clinical evaluations by a skilled practitioner (physician or veterinarian) to determine subsequent therapy. Hence, following treatment the practitioners will evaluate any improvement in the treatment of the pulmonary inflammation according to standard methodologies. Such evaluation will aid and inform in evaluating whether to increase, reduce or continue a particular treatment dose, mode of administration, etc. The term “therapeutic composition” refers to any compounds administered to treat or prevent a disease. It will be understood that the subject to which a compound (e.g., an antibody) of the invention is administered need not suffer from a specific traumatic state. Indeed, the compounds (e.g., antibodies) of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

TABLE 1
Phosphorylation Sites

| | A | | | D | | |
|---|---|---|---|---|---|---|
| | Protein | B | C | Phospho- | E | H |
| 1 | Name | Accession No. | Protein Type | Residue | Phsophorylation Site Sequence | SEQ ID NO |
| 2 | G3BP-1 | NP_005745.1 | Adaptor/scaffold | Y125 | FMQTFVLAPEGSVANKFyVHNDIFR | SEQ ID NO: 1 |
| 3 | G3BP-2 | NP_036429.2 | Adaptor/scaffold | Y56 | NSSYVHGGVDASGKPQEAVyGQNDIHHK | SEQ ID NO: 2 |
| 4 | Gab2 | NP_036428.1 | Adaptor/scaffold | Y10 | MSGDPDVLEyYKNDHSK | SEQ ID NO: 3 |
| 5 | Gab3 | NP_542179.1 | Adaptor/scaffold | Y183 | SESELLFLPDyLVLSNCETGR | SEQ ID NO: 4 |
| 6 | Gab3 | NP_542179.1 | Adaptor/scaffold | Y506 | FFANPVSREDEESyIEMEEHR | SEQ ID NO: 5 |
| 7 | GKAP1 | NP_079487.2 | Adaptor/scaffold | Y141 | LEyEEHKKEYEDAENTSTQSK | SEQ ID NO: 6 |
| 8 | GKAP1 | NP_079487.2 | Adaptor/scaffold | Y148 | LEYEEHKKEyEDAENTSTQSK | SEQ ID NO: 7 |
| 9 | GRAP2 | NP_004801.1 | Adaptor/scaffold | Y222 | yLQHHHFHQER | SEQ ID NO: 8 |
| 10 | GRASP | NP_859062.1 | Adaptor/scaffold | Y237 | LVHGLVVKDPSIyDTLESVR | SEQ ID NO: 9 |
| 11 | Grb10 | NP_005302.3 | Adaptor/scaffold | Y404 | YGMLLyQNYR | SEQ ID NO: 10 |
| 12 | HAP95 | NP_055186.2 | Adaptor/scaffold | Y125 | YDSyESCDSR | SEQ ID NO: 11 |
| 13 | HEFL | NP_065089.2 | Adaptor/scaffold | Y231 | RGGySTLPNPQKSEWIYDTPVSPGK | SEQ ID NO: 12 |
| 14 | HEFL | NP_065089.2 | Adaptor/scaffold | Y280 | NTPLTSFAEESRPHALPSSSSTFyNPPSGR | SEQ ID NO: 13 |
| 15 | HOOK3 | NP_115786.1 | Adaptor/scaffold | Y171 | ESPVSAGNDAyVDLDR | SEQ ID NO: 14 |
| 16 | HS1 | NP_005326.1 | Adaptor/scaffold | Y153 | SAVGFDYKGEVEKHTSQKDySR | SEQ ID NO: 15 |
| 17 | HS1 | NP_005326.1 | Adaptor/scaffold | Y175 | AALGyDYKGETEKHESQR | SEQ ID NO: 16 |
| 18 | HS1 | NP_005326.1 | Adaptor/scaffold | Y177 | AALGYDyKGETEKHESQRDYAK | SEQ ID NO: 17 |
| 19 | HS1 | NP_005326.1 | Adaptor/scaffold | Y190 | AALGYDYKGETEKHESQRDyAK | SEQ ID NO: 18 |
| 20 | IRS-1 | NP_005535.1 | Adaptor/scaffold | Y483 | GPSTLTAPNGHyILSR | SEQ ID NO: 19 |
| 21 | IRS-1 | NP_005535.1 | Adaptor/scaffold | Y695 | NDPNGYMMMSPSGGCSPDIGGSPSSSSSSSN | SEQ ID NO: 20 |
| | | | | | AVPSGTSyGK | |
| 22 | IRS-2 | NP_003740.2 | Adaptor/scaffold | Y814 | APyTCGGDSDQYVLMSSPVGR | SEQ ID NO: 21 |
| 23 | IRS-2 | NP_003740.2 | Adaptor/scaffold | Y978 | SPLSDyMNLDFSSPK | SEQ ID NO: 22 |
| 24 | ITSN2 | NP_006268.1 | Adaptor/scaffold | Y261 | SMSGyLSGFQAR | SEQ ID NO: 23 |
| 25 | ITSN2 | NP_006268.1 | Adaptor/scaffold | Y38 | QFDNLKPSGGyITGDQAR | SEQ ID NO: 24 |
| 26 | ITSN2 | NP_006266.1 | Adaptor/scaffold | Y664 | ETYNTQQLALEQLyK | SEQ ID NO: 25 |
| 27 | kanada | NP_060628.1 | Adaptor/scaffold | Y720 | ETQTHENMSQLSEEEQNKDyQDCSK | SEQ ID NO: 26 |
| | ptin | | | | | |
| 28 | LAT | NP_055202.1 | Adaptor/scaffold | Y45 | LPGSYDSTSSDSLyPR | SEQ ID NO: 27 |
| 29 | LAX1 | NP_060243.2 | Adaptor/scaffold | Y93 | NIyDILPWRQEDLGR | SEQ ID NO: 28 |
| 30 | LPP | NP_005569.1 | Adaptor/scaffold | Y287 | GGMDYAYIPPPGLQPEPGyGYAPNQGR | SEQ ID NO: 29 |
| 31 | LPP | NP_005569.1 | Adaptor/scaffold | Y289 | GGMDYAYIPPPGQGPQPGYGyAPNQGR | SEQ ID NO: 30 |
| 32 | LPP | NP_005569.1 | Adaptor/scaffold | Y296 | yYEGYYAAGPGYGGRDNSDPTYGQQGHPNTW | SEQ ID NO: 31 |
| | | | | | KR | |
| 33 | LPP | NP_005569.1 | Adaptor/scaffold | Y332 | REPGyTPPGAGNQNPPGMYPVTGPK | SEQ ID NO: 32 |
| 34 | MACF1 | NP_036222.3 | Adaptor/scaffold | Y3585 | yADITVTSSKALR | SEQ ID NO: 33 |
| 35 | FLOT2 | NP_004466.2 | Adhesion or | Y241 | TAEAQLAyELQGAR | SEQ ID NO: 34 |
| | | | extracellular matrix | | | |
| | | | protein | | | |
| 36 | HSPG2 | NP_005520.4 | Adhesion or | Y616 | yNVRYELAR | SEQ ID NO: 35 |
| | | | extracellular matrix | | | |
| | | | protein | | | |
| 37 | HSPG2 | NP_005520.4 | Adhesion or | Y620 | YNVRyELAR | SEQ ID NO: 36 |
| | | | extracellular matrix | | | |
| | | | protein | | | |
| 38 | KAL1 | NP_000207.2 | Adhesion or | Y259 | WyQFRVAAVNVHGTR | SEQ ID NO: 37 |
| | | | extracellular matrix | | | |
| | | | protein | | | |
| 39 | LAMA1 | NP_005550.2 | Adhesion or | Y490 | CKPGFyNLKEKNPR | SEQ ID NO: 38 |
| | | | extracellular matrix | | | |
| | | | protein | | | |
| 40 | LTBP4 | NP_003564.2 | Adhesion or | Y230 | AEAAAPyTVLAQSAPREDGYSDASGFGYCFR | SEQ ID NO: 39 |
| | | | extracellular matrix | | | |
| | | | protein | | | |
| 41 | LTBP4 | NP_003564.2 | Adhesion or | Y243 | AEAAAPYTVLAQSAPREDGySDASGFGYCFR | SEQ ID NO: 40 |
| | | | extracellular matrix | | | |
| | | | protein | | | |
| 42 | LTBP4 | NP_003564.2 | Adhesion or | Y251 | AEAAAPYTVLAQSAPREDGYSDASGFGyCFR | SEQ ID NO: 41 |
| | | | extracellular matrix | | | |
| | | | protein | | | |
| 43 | GRP94 | NP_003290.1 | Apoptosis | Y652 | LTESPCALVASQyGWSGNMER | SEQ ID NO: 42 |
| 44 | GRP94 | NP_003290.1 | Apoptosis | Y667 | AQAyQTGKDISTNYYASQK | SEQ ID NO: 43 |
| 45 | GRP94 | NP_003290.1 | Apoptosis | Y677 | AQAYQTGKDISTNyYASQK | SEQ ID NO: 44 |
| 46 | MMRP19 | NP_057041.1 | Apoptosis | Y57 | HGDEIyIAPSGVQK | SEQ ID NO: 45 |
| 47 | KAB1 | NP_055627.2 | Cell cycle regulation | Y1291 | IKEQEDyIR | SEQ ID NO: 46 |
| 48 | KAB1 | NP_055627.2 | Cell cycle regulation | Y177 | GTPLyGQPSWWGDDEVDEK | SEQ ID NO: 47 |
| 49 | KAB1 | NP_055627.2 | Cell cycle regulation | Y532 | KMIDKVFGVDDNQDyNRPVINEK | SEQ ID NO: 48 |
| 50 | KI-67 | NP_002408.3 | Cell cycle regulation | Y340 | AVGASFPLyEPAK | SEQ ID NO: 49 |
| 51 | MAD2L1 | NP_002349.1 | Cell cycle regulation | Y199 | VNSMVAyKIPVND | SEQ ID NO: 50 |
| 52 | MCM6 | NP_005906.2 | Cell cycle regulation | Y276 | VSGVDGyETEGIR | SEQ ID NO: 51 |
| 53 | MCM6 | NP_005906.2 | Cell cycle regulation | Y810 | GSTEGSESYEEDPyLVVNPNYLLED | SEQ ID NO: 52 |
| 54 | HDJ2 | NP_001530.1 | Chaperone | Y311 | CVLNEGMPIyR | SEQ ID NO: 53 |
| 55 | HSC70 | NP_006588.1 | Chaperone | Y107 | VQVEyKGETK | SEQ ID NO: 54 |
| 56 | HSC70 | NP_006588.1 | Chaperone | Y525 | MVQEAEKyKAEDEKQR | SEQ ID NO: 55 |
| 57 | Hsp105 | NP_006635.2 | Chaperone | Y643 | NAVEEYVyEFRDKLCGPYEK | SEQ ID NO: 56 |
| | alpha | | | | | |
| 58 | Hsp105 | NP_006635.2 | Chaperone | Y677 | LLTETEDWLyEEGEDQAK | SEQ ID NO: 57 |
| | alpha | | | | | |
| 59 | Hsp105 | NP_006635.2 | Chaperone | Y89 | ENLSyDLVPLK | SEQ ID NO: 58 |
| | alpha | | | | | |
| 60 | HSP70 | NP_005337.1 | Chaperone | Y15 | AAAIGIDLGTTySCVGVFQHGK | SEQ ID NO: 59 |
| 61 | LTBP1 | NP_000618.2 | Chaperone | Y409 | EICPGGMGyTVSGVHRRR | SEQ ID NO: 60 |
| 62 | H2B.1A | NP_003516.1 | Chromatin, DNA- | Y38 | KESySVYVYK | SEQ ID NO: 61 |
| | | | binding, DNA repair | | | |
| | | | or DNA replication | | | |
| | | | protein | | | |
| 63 | HIRIP3 | NP_003600.2 | Chromatin, DNA- | Y111 | FNSESESGSEASSPDyFGPPAK | SEQ ID NO: 62 |
| | | | binding, DNA repair | | | |
| | | | or DNA replication | | | |
| | | | protein | | | |
| 64 | HIVEP2 | NP_006725.3 | Chromatin, DNA- | Y1788 | SNEDyVYVR | SEQ ID NO: 63 |
| | | | binding, DNA repair | | | |
| | | | or DNA replication | | | |
| | | | protein | | | |
| 65 | HIVEP2 | NP_006725.3 | Chromatin, DNA- | Y638 | VGYDyDVCR | SEQ ID NO: 64 |
| | | | binding, DNA repair | | | |
| | | | or DNA replication | | | |
| | | | protein | | | |
| 66 | hnRNP | NP_112740.1 | Chromatin, DNA- | Y126 | QHPPADSSVTMEDMNEySNIEEFAEGSK | SEQ ID NO: 65 |
| | D-like | | binding, DNA repair | | | |
| | | | or DNA replication | | | |
| | | | protein | | | |
| 67 | Ku70 | NP_001460.1 | Chromatin, DNA- | Y103 | NIyVLQELDNPGAKR | SEQ ID NO: 66 |
| | | | binding, DNA repair | | | |
| | | | or DNA replication | | | |
| | | | protein | | | |
| 68 | Ku70 | NP_001460.1 | Chromatin, DNA- | Y530 | LGSLVDEFKELVyPPDYNPEGK | SEQ ID NO: 67 |
| | | | binding, DNA repair | | | |
| | | | or DNA replication | | | |
| | | | protein | | | |
| 69 | MCM3 | NP_002379.2 | Chromatin, DNA- | Y651 | TVDLQDAEEAVELVQyAYFK | SEQ ID NO: 68 |
| | | | binding, DNA repair | | | |
| | | | or DNA replication | | | |
| | | | protein | | | |
| 70 | MCM4 | NP_005905.2 | Chromatin, DNA- | Y522 | SQLLQyVYNLVPR | SEQ ID NO: 69 |
| | | | binding, DNA repair | | | |
| | | | or DNA replication | | | |
| | | | protein | | | |
| 71 | MCM7 | NP_005907.3 | Chromatin, DNA- | Y492 | CSILAAANPAyGR | SEQ ID NO: 70 |
| | | | binding, DNA repair | | | |
| | | | or DNA replication | | | |
| | | | protein | | | |
| 72 | MCM7 | NP_005907.3 | Chromatin, DNA- | Y600 | EAWASKDATyTSAR | SEQ ID NO: 71 |
| | | | binding, DNA repair | | | |
| | | | or DNA replication | | | |
| | | | protein | | | |
| 73 | FLNA | NP_001447.2 | Cytoskeletal protein | Y1755 | QQLAPQYTyAQGGQ | SEQ ID NO: 72 |
| 74 | FLNA | NP_001447.2 | Cytoskeletal protein | Y731 | DNGNGTySCSYVPR | SEQ ID NO: 73 |
| 75 | FLNA | NP_001447.2 | Cytoskeletal protein | Y735 | DNGNGTYSCSyVPR | SEQ ID NO: 74 |
| 76 | FLNB | NP_001448.2 | Cytoskeletal protein | Y704 | NRMDGTyACSYTPVK | SEQ ID NO: 75 |
| 77 | FLNB | NP_001448.2 | Cytoskeletal protein | Y708 | NRMDGTYACSyTPVK | SEQ ID NO: 76 |
| 78 | FLNC | NP_001449.3 | Cytoskeletal protein | Y2683 | TPCEEVyVK | SEQ ID NO: 77 |
| 79 | FRMD4B | XP_114303.4 | Cytoskeletal protein | Y1096 | SFHEDEVDRVPHNPyATLR | SEQ ID NO: 78 |
| 80 | GCP3 | NP_006313.1 | Cytoskeletal protein | Y256 | DILyVFQGIDGK | SEQ ID NO: 79 |
| 81 | Golgin-84 | NP_005104.2 | Cytoskeletal protein | Y544 | LKQEFHyIEEDLYRTK | SEQ ID NO: 80 |
| 82 | KRT1 | NP_006112.3 | Cytoskeletal protein | Y539 | GGGGGGYGSGGSSYGSGGGSyGSGGGGGGGR | SEQ ID NO: 81 |
| 83 | KRT1 | NP_001662.3 | Cytoskeletal protein | Y639 | FVSTTySGVTR | SEQ ID NO: 82 |
| 84 | KRTAP | NP_853635.1 | Cytoskeletal protein | Y20 | MCGSYYGNYYGDHGYGCCGyEGLGYGYGSLR | SEQ ID NO: 83 |
| | 6-2 | | | | | |
| 85 | KRTAP | NP_853635.1 | Cytoskeletal protein | Y25 | MCGSYYGNYYGDHGYGCCGYEGLGyGYGSLR | SEQ ID NO: 84 |
| | 6-2 | | | | | |
| 86 | KRTAP | NP_853635.1 | Cytoskeletal protein | Y9 | MCGSYYGNyYGDHGYGCCGYEGLGYGYGSLR | SEQ ID NO: 85 |
| | 6-2 | | | | | |
| 87 | Lasp-1 | NP_006139.1 | Cytoskeletal protein | Y183 | AQSyGGYKEPAAPV | SEQ ID NO: 86 |
| 88 | Lasp-1 | NP_006139.1 | Cytoskeletal protein | Y52 | KPyCNAHYPK | SEQ ID NO: 87 |
| 89 | Lasp-1 | NP_006139.1 | Cytoskeletal protein | Y57 | KPYCNAHyPK | SEQ ID NO: 88 |
| 90 | L-plastin | NP_002289.1 | Cytoskeletal protein | Y118 | EGICAIGGTSEQSSVGTQHSySEEEKYAFVN | SEQ ID NO: 89 |
| | | | | | WINK | |
| 91 | L-plastin | NP_002289.1 | Cytoskeletal protein | Y299 | AyYHLLEQVAPK | SEQ ID NO: 90 |
| 92 | L-plastin | NP_002289.1 | Cytoskeletal protein | Y374 | yPALHKPENQDIDWGALEGETR | SEQ ID NO: 91 |
| 93 | LST1 | NP_995310.1 | Cytoskeletal protein | Y42 | GTKEDPRADyACIAENKPT | SEQ ID NO: 92 |
| 94 | MAP1A | NP_002364.5 | Cytoskeletal protein | Y177 | LGIQAEPLyRVVSNTIEPLTLFHK | SEQ ID NO: 93 |
| 95 | MAP1A | NP_002364.5 | Cytoskeletal protein | Y681 | AEGFyQK | SEQ ID NO: 94 |
| 96 | MAP1A | NP_002364.5 | Cytoskeletal protein | Y958 | LCSQyGTPVFSAPGHALHPGEPALGEAEER | SEQ ID NO: 95 |
| 97 | MAP2 | NP_002365.3 | Cytoskeletal protein | Y592 | SIEPGSDyYELSDTR | SEQ ID NO: 96 |
| 98 | MAP4 | NP_002366.2 | Cytoskeletal protein | Y47 | TDyIPLLDVDEK | SEQ ID NO: 97 |
| 99 | GAPDH | NP_002037.2 | Enzyme, misc. | Y314 | LISWyDNEFGYSNR | SEQ ID NO: 98 |
| 100 | GAPDH | NP_002037.2 | Enzyme, misc. | Y320 | LISWYDNEFGySNRVVDLMAHMASKE | SEQ ID NO: 99 |
| 101 | GARS | NP_002038.2 | Enzyme, misc. | Y148 | GGVSGLyDFGPVGCAL | SEQ ID NO: 100 |
| 102 | GARS | NP_002038.2 | Enzyme, misc. | Y467 | SCyDLSCHAR | SEQ ID NO: 101 |
| 103 | GDE | NP_000019.2 | Enzyme, misc. | Y638 | SAyDALPSTTIVSMACCASGSTR | SEQ ID NO: 102 |
| 104 | GlnRS | NP_005042.1 | Enzyme, misc. | Y57 | EAATQAQQTLGSTIDKATGILLyGLASR | SEQ ID NO: 103 |
| 105 | GLO1 | NP_006699.2 | Enzyme, misc. | Y136 | GFGHIGIAVPDVySACKR | SEQ ID NO: 104 |
| 106 | GLUD1 | NP_005262.1 | Enzyme, misc. | Y451 | NLNHVSyGR | SEQ ID NO: 105 |
| 107 | GLUD1 | NP_005262.1 | Enzyme, misc. | Y512 | DIVHSGLAyTMER | SEQ ID NO: 106 |
| 108 | GMD | NP_001491.1 | Enzyme, misc. | Y84 | LHyGDLTDSTCLVK | SEQ ID NO: 107 |
| 109 | GLOGA7 | NP_057183.2 | Enzyme, misc. | Y54 | TLNNLyAEAEK | SEQ ID NO: 108 |
| 110 | GOT1 | NP_002070.1 | Enzyme, misc. | Y381 | HIyLLPSGR | SEQ ID NO: 109 |
| 111 | GOT1 | NP_002070.1 | Enzyme, misc. | Y400 | NLDyVATSIHEAVTK | SEQ ID NO: 110 |
| 112 | GOT2 | NP_002071.2 | Enzyme, misc. | Y96 | NLDKEyLPIGGLAEFCK | SEQ ID NO: 111 |
| 113 | GRHPR | NP_036335.1 | Enzyme, misc. | Y255 | GDVVNQDDLyQALASGK | SEQ ID NO: 112 |
| 114 | GSTP1 | NP_000843.1 | Enzyme, misc. | Y199 | AFLASPEyVNLPINGNGKQ | SEQ ID NO: 113 |
| 115 | GSTP1 | NP_000843.1 | Enzyme, misc. | Y64 | FQDGDLTLyQSNTILR | SEQ ID NO: 114 |
| 116 | HADHA | NP_000173.2 | Enzyme, misc. | Y724 | FVDLyGAQK | SEQ ID NO: 115 |
| 117 | HDAC2 | NP_001518.2 | Enzyme, misc. | Y453 | LHISPSNMTNQNTPEyMEK | SEQ ID NO: 116 |
| 118 | HDAC2 | NP_001518.2 | Enzyme, misc. | Y182 | SIRPDNMSEySK | SEQ ID NO: 117 |
| 119 | HDAC7 | NP_056216.1 | Enzyme, misc. | Y524 | TLPFTTGLIyDSVMLK | SEQ ID NO: 118 |
| 120 | helicase | NP_387467.2 | Enzyme, misc. | Y721 | TNHHSCLySAVK | SEQ ID NO: 119 |
| | B | | | | | |
| 121 | HELZ | NP_055692.2 | Enzyme, misc. | Y456 | SLTKSNyQSRLHDLLYIEEIAQYK | SEQ ID NO: 120 |
| 122 | HELZ | NP_055692.2 | Enzyme, misc. | Y465 | SLTKSNYQSRLHDLLyIEEIAQYK | SEQ ID NO: 121 |
| 123 | HIP14 | NP_056151.2 | Enzyme, misc. | Y67 | ATQyGIYER | SEQ ID NO: 122 |
| 124 | HIP14 | NP_056151.2 | Enzyme, misc. | Y70 | ATQYGIyER | SEQ ID NO: 123 |
| 125 | HMGCS1 | NP_002121.3 | Enzyme, misc. | Y213 | GTHMQHAYDFYKPDMLSEyPIVDGK | SEQ ID NO: 124 |
| 126 | IARS | NP_002152.2 | Enzyme, misc. | Y434 | NNDLCyWVPELVR | SEQ ID NO: 125 |
| 127 | IMP | NP_000875.2 | Enzyme, misc. | Y509 | TSSAQVEGGVHSLHSyEK | SEQ ID NO: 126 |
| | dehydro- | | | | | |
| | genase 2 | | | | | |
| 128 | KIAA0339 | NP_055527.1 | Enzyme, misc. | Y179 | GQQRMKyYELIVNGSYTPQTVPTGGKALSEK | SEQ ID NO: 127 |
| 129 | KIAA0339 | NP_055527.1 | Enzyme, misc. | Y180 | GQQRMKYyELIVNGSYTPQTVPTGGKALSEK | SEQ ID NO: 128 |
| 130 | KIAA0339 | NP_055527.1 | Enzyme, misc. | Y188 | GQQRMKYYELIVNGSyTPQTVPTGGKALSEK | SEQ ID NO: 129 |
| 131 | Ku80 | NP_066964.1 | Enzyme, misc. | Y416 | HNyECLVYVQLPFMEDLR | SEQ ID NO: 130 |
| 132 | LARS | NP_064502.9 | Enzyme, misc. | Y264 | QTGEGVGPQEyTLLK | SEQ ID NO: 131 |
| 133 | LDH-A | NP_005557.1 | Enzyme, misc. | Y172 | FRyLMGER | SEQ ID NO: 132 |
| 134 | LDH-B | NP_002291.1 | Enzyme, misc. | Y84 | IVADKDySVTANSK | SEQ ID NO: 133 |
| 135 | LIG3 | NP_002302.2 | Enzyme, misc. | Y767 | VNKIyYPDFIVPDPK | SEQ ID NO: 134 |
| 136 | LIG3 | NP_002302.2 | Enzyme, misc. | Y768 | VNKIYyPDFIVPDPK | SEQ ID NO: 135 |
| 137 | LSD1 | NP_055828.2 | Enzyme, misc. | Y363 | QKCPLyEANGQAVPKEKDEMVEQEFNR | SEQ ID NO: 136 |
| 138 | LSS | NP_002331.3 | Enzyme, misc. | Y130 | yLRSVQLPDGGWGLHIEDK | SEQ ID NO: 137 |
| 139 | MANBA | NP_005899.3 | Enzyme, misc. | Y161 | yQVPPDCPPLVQK | SEQ ID NO: 138 |
| 140 | MDH2 | NP_005909.2 | Enzyme, misc. | Y253 | AGAGSATLSMAyAGAR | SEQ ID NO: 139 |
| 141 | MDH2 | NP_005909.2 | Enzyme, misc. | Y80 | GyLGPEQLPDCLK | SEQ ID NO: 140 |
| 142 | MTHFD1 | NP_005947.2 | Enzyme, misc. | Y402 | STTTIGLVQALGAHLyQNVFACVR | SEQ ID NO: 141 |
| 143 | G- | NP_006487.1 | G protein or | Y61 | IIHEDGySEDECKQYK | SEQ ID NO: 142 |
| | alpha3(i) | | regulator | | | |
| 144 | G- | NP_000597.1 | G protein or | Y311 | SKIEDyFPEFAR | SEQ ID NO: 143 |
| | alpha-s | | regulator | | | |
| 145 | G- | NP_000507.1 | G protein or | Y360 | HYCyPHFTCAVDTENIR | SEQ ID NO: 144 |
| | alpha-s | | regulator | | | |
| 146 | G- | NP_002065.1 | G protein or | Y111 | SSWVMTCAYAPSGNyVACGGLDNICSIYNLK | SEQ ID NO: 145 |
| | beta(1) | | regulator | | | |
| 147 | GBF1 | NP_004184.1 | G protein or | Y1316 | GyTSDSEVYTDHGRPGK | SEQ ID NO: 146 |
| | | | regulator | | | |
| 148 | GBF1 | NP_004184.1 | G protein or | Y1323 | GYTSDSEVyTDHGRPGK | SEQ ID NO: 147 |
| | | | regulator | | | |
| 149 | GDI1 | NP_001484.1 | G protein or | Y224 | SPyLYPLYGLGELPQGFAR | SEQ ID NO: 148 |
| | | | regulator | | | |
| 150 | GDI1 | NP_001484.1 | G protein or | Y226 | SPYLyPLYGLGELPQGFAR | SEQ ID NO: 149 |
| | | | regulator | | | |
| 151 | GDI1 | NP_001484.1 | G protein or | Y229 | SPYLYPLyGLGELPQGFAR | SEQ ID NO: 150 |
| | | | regulator | | | |
| 152 | GDI2 | NP_001485.2 | G protein or | Y117 | GGKIyKVPSTEAEALASSLMGLFEK | SEQ ID NO: 151 |
| | | | regulator | | | |
| 153 | GDI2 | NP_001485.2 | G protein or | Y229 | YGKSPYLYPLyGLGELPQGFAR | SEQ ID NO: 152 |
| | | | regulator | | | |
| 154 | GIT1 | NP_054749.2 | G protein or | Y510 | DRQAFSMyEPGSALKPFGGPPGDELTTR | SEQ ID NO: 153 |
| | | | regulator | | | |
| 155 | Gnb3 | NP_002066.1 | G protein or | Y59 | GHLAKIyAMHWATDSK | SEQ ID NO: 154 |
| | | | regulator | | | |
| 156 | GPSM1 | NP_056412.2 | G protein or | Y127 | ALyNIGNVYHAK | SEQ ID NO: 155 |
| | | | regulator | | | |
| 157 | H-Ras-1 | NP_005334.1 | G protein or | Y157 | QGVEDAFyTLVR | SEQ ID NO: 156 |
| | | | regulator | | | |
| 158 | IPO8 | NP_006381.2 | G protein or | Y30 | IAAENELNQSyK | SEQ ID NO: 157 |
| | | | regulator | | | |
| 159 | IQGAP1 | NP_003861.1 | G protein or | Y133 | IFyPETTDIYDRKNMPR | SEQ ID NO: 158 |
| | | | regulator | | | |
| 160 | IQGAP1 | NP_003861.1 | G protein or | Y140 | IFYPETTDIyDRKNMPR | SEQ ID NO: 159 |
| | | | regulator | | | |
| 161 | IQGAP2 | NP_006624.2 | G protein or | Y1172 | LFEGENEHLSSMNNYLSETyQEFR | SEQ ID NO: 160 |
| | | | regulator | | | |
| 162 | IQGAP2 | NP_006624.2 | G protein or | Y1393 | TLEQTGHVSSENKyQDILNEIAK | SEQ ID NO: 161 |
| | | | regulator | | | |
| 163 | IQGAP2 | NP_006624.2 | G protein or | Y499 | TLETLLLPTANISDVDPAHAQHYQDVLyHAK | SEQ ID NO: 162 |
| | | | regulator | | | |
| 164 | IQGAP2 | NP_006624.2 | G protein or | Y579 | SSTSNANDIIPECADKyYDALVK | SEQ ID NO: 163 |
| | | | regulator | | | |
| 165 | IQGAP2 | NP_006624.2 | G protein or | Y580 | SSTSNANDIIPECADKYyDALVK | SEQ ID NO: 164 |
| | | | regulator | | | |
| 166 | IQGAP2 | NP_006624.2 | G protein or | Y611 | KYDyYYNTDSK | SEQ ID NO: 165 |
| | | | regulator | | | |
| 166 | IQGAP2 | NP_006624.2 | G protein or | Y612 | KYDYyYNTDSK | SEQ ID NO: 166 |
| | | | regulator | | | |
| 168 | IQGAP2 | NP_006624.2 | G protein or | Y613 | KYDYYyNTDSK | SEQ ID NO: 167 |
| | | | regulator | | | |
| 169 | IQGAP2 | NP_006624.2 | G protein or | Y770 | ARDDyKTLVGSENPPLTVIR | SEQ ID NO: 168 |
| | | | regulator | | | |
| 170 | IQGAP2 | NP_006624.2 | G protein or | Y93 | KIyDVEQTR | SEQ ID NO: 169 |
| | | | regulator | | | |
| 171 | MgcRacGAP | NP_037409.2 | G protein or | Y241 | TTVTVPNDGGPIEAVSTIETVPyWTR | SEQ ID NO: 170 |
| | | | regulator | | | |
| 172 | ITIH2 | NP_002207.2 | Inhibitor protein | Y277 | ETAVDGELVVLyDVKR | SEQ ID NO: 171 |
| 173 | MPP1 | NP_002427.1 | Kinase (non-protein) | Y331 | KSEEDGKEyHFISTEEMTR | SEQ ID NO: 172 |
| 174 | MPP1 | NP_002427.1 | Kinase (non-protein) | Y429 | SQYAHyFDLSLVNNGVDETLKK | SEQ ID NO: 173 |
| 175 | HCCS | NP_005324.2 | Mitochondrial protein | Y63 | AYEyVECPIR | SEQ ID NO: 174 |
| 176 | KSPE1 | NP_002148.1 | Mitochondrial protein | Y88 | VVLDDKDyFLFRDGDILGK | SEQ ID NO: 175 |
| 177 | MRPL4 | NP_057040.2 | Mitochondrial protein | Y162 | GPTSYyYMLPMK | SEQ ID NO: 176 |
| 178 | KIF23 | NP_004847.2 | Motor or contractile | Y582 | TTTIyEEDKR | SEQ ID NO: 177 |
| | | | protein | | | |
| 179 | KIFC1 | NP_002254.1 | Motor or contractile | Y622 | LTyLLQNSLGGSAK | SEQ ID NO: 178 |
| | | | protein | | | |
| 180 | kinesin | NP_073733.1 | Motor or contractile | Y292 | TLGKDHPAVAATLNNLAVLyGK | SEQ ID NO: 179 |
| | light | | protein | | | |
| | chain 2 | | | | | |
| 181 | kinesin | NP_073733.1 | Motor or contractile | Y346 | QLSNLALLCQNQGKAEEVEYyYRR | SEQ ID NO: 180 |
| | light | | protein | | | |
| | chain 2 | | | | | |
| 182 | kinesin | NP_073733.1 | Motor or contractile | Y347 | QLSNLALLCQNQGKAEEVEYYyRR | SEQ ID NO: 181 |
| | light | | protein | | | |
| | chain 2 | | | | | |
| 183 | kinesin | NP_073733.1 | Motor or contractile | Y431 | DSAPyGEYGSWYK | SEQ ID NO: 182 |
| | light | | protein | | | |
| | chain 2 | | | | | |
| 184 | KNS2 | NP_005543.2 | Motor or contractile | Y271 | DQNKyKDAANLLNDALALIR | SEQ ID NO: 183 |
| | | | protein | | | |
| 185 | KNS2 | NP_005543.2 | Motor or contractile | Y307 | TLGKDHPAVAATLNNLAVLyGK | SEQ ID NO: 184 |
| | | | protein | | | |
| 186 | KNS2 | NP_005543.2 | Motor or contractile | Y360 | QLNNLALLCQNQGKYEEVEyYYQR | SEQ ID NO: 185 |
| | | | protein | | | |
| 187 | MTMR6 | NP_004676.3 | Phosphatase | Y261 | GYENEDNySNIR | SEQ ID NO: 186 |
| 188 | MTMR6 | NP_004676.3 | Phosphatase | Y595 | ySEYAEEFSK | SEQ ID NO: 187 |
| 189 | MTMR6 | NP_004676.3 | Phosphatase | Y598 | TIEGSSPADNRYSEyAEEFSKSEPAVVSLEY | SEQ ID NO: 188 |
| | | | | | GVAR | |
| 190 | MTMR6 | NP_004676.3 | Phosphatase | Y614 | TIEGSSPADNRYSEYAEEFSKSEPAVVSLEy | SEQ ID NO: 189 |
| | | | | | GVAR | |
| 191 | IRAP | NP_005566.2 | Protease | Y46 | EPCLHPLEPDEVEyEPR | SEQ ID NO: 190 |
| 192 | MKK6 | NP_002749.2 | protein kinase, dual- | Y64 | MELGRGAyGVVEK | SEQ ID NO: 191 |
| | | | specificity | | | |
| 193 | MOBKL1A | NP_775739.1 | protein kinase, | Y26 | KNIPEGSHQyELLK | SEQ ID NO: 192 |
| | | | regulatory subunit | | | |
| 194 | GAK | NP_005246.1 | protein kinase, Ser/ | Y367 | GPPPPVGPAGSGYSGGLALAEyDQPYGGFLD | SEQ ID NO: 193 |
| | | | Thr (non-receptor) | | ILR | |
| 195 | GCK | NP_004570.2 | protein kinase, Ser/ | Y27 | VGAGTyGDVYK | SEQ ID NO: 194 |
| | | | Thr (non-receptor) | | | |
| 196 | GSK3- | NP_063937.2 | protein kinase, Ser/ | Y134 | VIGNGSFGVVyQAR | SEQ ID NO: 195 |
| | alpha | | Thr (non-receptor) | | | |
| 197 | GSK3- | NP_002084.2 | protein kinase, Ser/ | Y71 | VIGNGSFGVVyQAK | SEQ ID NO: 196 |
| | beta; | | Thr (non-receptor) | | | |
| | GSK3- | | | | | |
| | beta | | | | | |
| 198 | HGK | NP_004825.2 | protein kinase, Ser/ | Y1154 | SGGSSQVyFMTLGR | SEQ ID NO: 197 |
| | | | Thr (non-receptor) | | | |
| 199 | HGK | NP_004825.2 | protein kinase, Ser/ | Y86 | NIATYyGAFIK | SEQ ID NO: 198 |
| | | | Thr (non-receptor) | | | |
| 200 | KHS1 | NP_006566.2 | protein kinase, Ser/ | Y31 | VGSGTyGDVYK | SEQ ID NO: 199 |
| | | | Thr (non-receptor) | | | |
| 201 | KHS1 | NP_006566.2 | protein kinase, Ser/ | Y35 | VGSGTYGDVyKAR | SEQ ID NO: 200 |
| | | | Thr (non-receptor) | | | |
| 202 | KHS2 | NP_003609.2 | protein kinase, Ser/ | Y366 | ETEPHHELPDSDGFLDSSEEIyYTAR | SEQ ID NO: 201 |
| | | | Thr (non-receptor) | | | |
| 203 | KHS2 | NP_003609.2 | protein kinase, Ser/ | Y379 | SNLDLQLEyGQGHQGGYFLGANK | SEQ ID NO: 202 |
| | | | Thr (non-receptor) | | | |
| 204 | KHS2 | NP_003609.2 | protein kinase, Ser/ | Y387 | SNLDLQLEYGQGHQGGyFLGANK | SEQ ID NO: 203 |
| | | | Thr (non-receptor) | | | |
| 205 | LRRK2 | NP_940980.2 | protein kinase, Ser/ | Y707 | VAMDDyLKNVMLER | SEQ ID NO: 204 |
| | | | Thr (non-receptor) | | | |
| 206 | MAK | NP_005897.1 | protein kinase, Ser/ | Y480 | QYyLKQAR | SEQ ID NO: 205 |
| | | | Thr (non-receptor) | | | |
| 207 | MAPKAPK3 | NP_004626.1 | protein kinase, Ser/ | Y76 | LLyDSPK | SEQ ID NO: 206 |
| | | | Thr (non-receptor) | | | |
| 208 | MAST1 | NP_055790.1 | protein kinase, Ser/ | Y182 | SPSSYDNEIVMMNHVyKER | SEQ ID NO: 207 |
| | | | Thr (non-receptor) | | | |
| 209 | MEKK1 | XP_042066.10 | protein kinase, Ser/ | Y1574 | yGAFKESVVINYTEQLLR | SEQ ID NO: 208 |
| | | | Thr (non-receptor) | | | |
| 210 | MEKK2 | NP_006600.3 | protein kinase, Ser/ | Y240 | AQSyPDNHQEFSDYDNPIFEKFGK | SEQ ID NO: 209 |
| | | | Thr (non-receptor) | | | |
| 211 | MEKK2 | NP_006600.3 | protein kinase, Ser/ | Y250 | AQSYPDNHQEFSDyDNPIFEKFGK | SEQ ID NO: 210 |
| | | | Thr (non-receptor) | | | |
| 212 | MEKK6 | NP_004663.3 | protein kinase, Ser/ | Y717 | YLGSASQGGyLK | SEQ ID NO: 211 |
| | | | Thr (non-receptor) | | | |
| 213 | MELK | NP_055606.1 | protein kinase, Ser/ | Y269 | NLLNHPWIMQDYNyPVEWQSK | SEQ ID NO: 212 |
| | | | Thr (non-receptor) | | | |
| 214 | MINK | NP_056531.1 | protein kinase, Ser/ | Y1082 | QGWTTVGMEGCGHyR | SEQ ID NO: 213 |
| | | | Thr (non-receptor) | | | |
| 215 | MSK1 | NP_004746.2 | protein kinase, Ser/ | Y60 | VLGTGAyGKVFLVR | SEQ ID NO: 214 |
| | | | Thr (non-receptor) | | | |
| 216 | MST1 | NP_006273.1 | protein kinase, Ser/ | Y433 | IPQDGDyEFLK | SEQ ID NO: 215 |
| | | | Thr (non-receptor) | | | |
| 217 | MST1 | NP_006273.1 | protein kinase, Ser/ | Y45 | LGEGSYGSVyK | SEQ ID NO: 216 |
| | | | Thr (non-receptor) | | | |
| 218 | Hck | NP_002101.2 | protein kinase, Tyr | Y330 | LHAVVTKEPIyIITEFMAK | SEQ ID NO: 217 |
| | | | (non-receptor) | | | |
| 219 | ITK | NP_005537.3 | protein kinase, Tyr | Y146 | LATGCAQyDPTK | SEQ ID NO: 218 |
| | | | (non-receptor) | | | |
| 220 | ITK | NP_005537.3 | protein kinase, Tyr | Y198 | RNEEyCLLDSSEIHWWR | SEQ ID NO: 219 |
| | | | (non-receptor) | | | |
| 221 | ITK | NP_005537.3 | protein kinase, Tyr | Y273 | TAGTyTVSVFTK | SEQ ID NO: 220 |
| | | | (non-receptor) | | | |
| 222 | Jak2 | NP_004963.1 | protein kinase, Tyr | Y382 | LTADAHHyLCK | SEQ ID NO: 221 |
| | | | (non-receptor) | | | |
| 223 | Jak2 | NP_004963.1 | protein kinase, Tyr | Y423 | KAGNQTGLyVLR | SEQ ID NO: 222 |
| | | | (non-receptor) | | | |
| 224 | Jak2 | NP_004963.1 | protein kinase, Tyr | Y435 | RCSPKDFNKyFL | SEQ ID NO: 223 |
| | | | (non-receptor) | | | |
| 225 | Lck | NP_005347.3 | protein kinase, Tyr | Y263 | LGAGQFGEVWMGyYNGHTK | SEQ ID NO: 224 |
| | | | (non-receptor) | | | |
| 226 | Lck | NP_005347.3 | protein kinase, Tyr | Y264 | LGAGQFGEVWMGYyNGHTK | SEQ ID NO: 225 |
| | | | (non-receptor) | | | |
| 227 | Lck | NP_005347.3 | protein kinase, Tyr | Y414 | FPIKWTAPEAINyGTFTIK | SEQ ID NO: 226 |
| | | | (non-receptor) | | | |
| 228 | Lyn | NP_002341.1 | protein kinase, Tyr | Y306 | LyAVVTR | SEQ ID NO: 227 |
| | | | (non-receptor) | | | |
| 229 | Lyn | NP_002341.1 | protein kinase, Tyr | Y316 | LYAVVTREEPIyIITEYMAK | SEQ ID NO: 228 |
| | | | (non-receptor) | | | |
| 230 | Lyn | NP_002341.1 | protein kinase, Tyr | Y460 | TNADVMTALSQGyR | SEQ ID NO: 229 |
| | | | (non-receptor) | | | |
| 231 | Lyn | NP_002341.1 | protein kinase, Tyr | Y501 | EKAEERPTFDYLQSVLDDFyTATEGQYQQQP | SEQ ID NO: 230 |
| | | | (non-receptor) | | | |
| 232 | Kit | NP_000213.1 | protein kinase, Tyr | Y609 | VVEATAyGLIK | SEQ ID NO: 231 |
| | | | (receptor) | | | |
| 233 | Lmr2 | NP_055731.2 | protein kinase, Tyr | Y1100 | GTEVTPETFTAGSQGSyR | SEQ ID NO: 232 |
| | | | (receptor) | | | |
| 234 | GLE1L | NP_001490.1 | Receptor, channel, | Y652 | MLILIKEDyFPR | SEQ ID NO: 233 |
| | | | transporter or cell | | | |
| | | | surface protein | | | |
| 235 | GluR- | NP_060021.1 | Receptor, channel, | Y780 | GYGIALQHGSPyRDLFSQR | SEQ ID NO: 234 |
| | delta1 | | transporter or cell | | | |
| | | | surface protein | | | |
| 236 | GPI- | NP_005889.3 | Receptor, channel, | Y283 | AEPEPAEEyEQSE | SEQ ID NO: 235 |
| | anchored | | transporter or cell | | | |
| | protein | | surface protein | | | |
| | p137 | | | | | |
| 237 | GPI- | NP_005889.3 | Receptor, channel, | Y541 | TLKQQNQyQASYNQSFFSSQPHQVE | SEQ ID NO: 236 |
| | anchored | | transporter or cell | | | |
| | protein | | surface protein | | | |
| | p137 | | | | | |
| 238 | GPR92 | NP_065133.1 | Receptor, channel, | Y29 | LVVySLVLAAGLPLNAL | SEQ ID NO: 237 |
| | | | transporter or cell | | | |
| | | | surface protein | | | |
| 239 | Hbb-b1 | NP_000510.1 | Receptor, channel, | Y146 | VVAGVANALAHKyH | SEQ ID NO: 238 |
| | | | transporter or cell | | | |
| | | | surface protein | | | |
| 240 | Hbb-b1 | NP_000510.1 | Receptor, channel, | Y36 | LLVVyPWTQR | SEQ ID NO: 239 |
| | | | transporter or cell | | | |
| | | | surface protein | | | |
| 241 | HLAA | AAX51797.1 | Receptor, channel, | Y344 | KGGSySQAASSDSAQGSDVSLTACKV | SEQ ID NO: 240 |
| | | | transporter or cell | | | |
| | | | surface protein | | | |
| 242 | HLAB | NP_005505.2 | Receptor, channel, | Y344 | GGSySQAACSDSAQGSDVSLTA | SEQ ID NO: 241 |
| | | | transporter or cell | | | |
| | | | surface protein | | | |
| 243 | HMHA1 | NP_036424.2 | Receptor, channel, | Y295 | NMAKyMK | SEQ ID NO: 242 |
| | | | transporter or cell | | | |
| | | | surface protein | | | |
| 244 | HMHA1 | NP_036424.2 | Receptor, channel, | Y466 | NKAEEAMATyR | SEQ ID NO: 243 |
| | | | transporter or cell | | | |
| | | | surface protein | | | |
| 245 | Icln | NP_001284.1 | Receptor, channel, | Y200 | LEGMLSQSVSSQyNMAGVR | SEQ ID NO: 244 |
| | | | transporter or cell | | | |
| | | | surface protein | | | |
| 246 | IFITM3 | NP_066362.1 | Receptor, channel, | Y20 | NSGQPPNyEMLKEEHE | SEQ ID NO: 245 |
| | | | transporter or cell | | | |
| | | | surface protein | | | |
| 247 | IL2RG | NP_000197.1 | Receptor, channel, | Y303 | NLEDLVTEyHGNFSAWSGVSK | SEQ ID NO: 246 |
| | | | transporter or cell | | | |
| | | | surface protein | | | |
| 248 | IL2RG | NP_000197.1 | Receptor, channel, | Y325 | GLAESLQPDySER | SEQ ID NO: 247 |
| | | | transporter or cell | | | |
| | | | surface protein | | | |
| 249 | IL2RG | NP_000197.1 | Receptor, channel, | Y357 | GGALGEGPGASPCNQHSPyWAPPCYTLKPET | SEQ ID NO: 248 |
| | | | transporter or cell | | | |
| | | | surface protein | | | |
| 250 | IL6R | NP_000556.1 | Receptor, channel, | Y457 | SPyDISNTDYFFPR | SEQ ID NO: 249 |
| | | | transporter or cell | | | |
| | | | surface protein | | | |
| 251 | IL6R | NP_000556.1 | Receptor, channel, | Y464 | SPYDISNTDyFFPR | SEQ ID NO: 250 |
| | | | transporter or cell | | | |
| | | | surface protein | | | |
| 252 | KCNK5 | NP_003731.1 | Receptor, channel, | Y347 | TSGGGETGPGPGLGPQGGGLPALPPSLVPLV | SEQ ID NO: 251 |
| | | | transporter or cell | | VySK | |
| | | | surface protein | | | |
| 253 | KPNA1 | NP_002255.2 | Receptor, channel, | Y476 | LIEEAyGLDK | SEQ ID NO: 252 |
| | | | transporter or cell | | | |
| | | | surface protein | | | |
| 254 | Kv-beta2 | NP_003627.1 | Receptor, channel, | Y184 | AMTHVINQGMAMyWGTSR | SEQ ID NO: 253 |
| | | | transporter or cell | | | |
| | | | surface protein | | | |
| 255 | LANCL1 | NP_006046.1 | Receptor, channel, | Y250 | LHSLVKPSVDyVCQLK | SEQ ID NO: 254 |
| | | | transporter or cell | | | |
| | | | surface protein | | | |
| 256 | latro- | NP_055736.2 | Receptor, channel, | Y1436 | NPLQGYyQVR | SEQ ID NO: 255 |
| | philin 1 | | transporter or cell | | | |
| | | | surface protein | | | |
| 257 | LBP | NP_004130.2 | Receptor, channel, | Y459 | LAEGFPLPLLKRVQLyDLGLQIHK | SEQ ID NO: 256 |
| | | | transporter or cell | | | |
| | | | surface protein | | | |
| 258 | LILRB1 | NP_006660.3 | Receptor, channel, | Y562 | SPHDEDPQAVTyAEVK | SEQ ID NO: 257 |
| | | | transporter or cell | | | |
| | | | surface protein | | | |
| 259 | MCEMP1 | NP_777578.2 | Receptor, channel, | Y38 | NQGADHPDyENITLAFK | SEQ ID NO: 258 |
| | | | transporter or cell | | | |
| | | | surface protein | | | |
| 260 | hnRNP2H9 | NP_036339.1 | RNA binding protein | Y159 | RGGDGYDGGYGGFDDYGGYNNyGYGNDGFDD | SEQ ID NO: 259 |
| | | | | | R | |
| 261 | hnRNP2H9 | NP_036339.1 | RNA binding protein | Y331 | GGGGSGGyYGQGGMSGGGWR | SEQ ID NO: 260 |
| 262 | hnRNP2H9 | NP_036339.1 | RNA binding protein | Y332 | GGGGSGGYyGQGGMSGGGWR | SEQ ID NO: 261 |
| 263 | hnRNP | NP_002128.1 | RNA binding protein | Y275 | GGGGPGYGNQGGGYGGGyDNY | SEQ ID NO: 262 |
| | A2/B1 | | | | | |
| 264 | hnRNP | NP_002128.1 | RNA binding protein | Y341 | GNFGGSRNMGGPYGGGNYGPGGSGGSGGYGG | SEQ ID NO: 263 |
| | A2/B1 | | | | RSRy | |
| 265 | hnRNPF | NP_004957.1 | RNA binding protein | Y253 | MRPGAYSTGYGGYEEYSGLSDGyGFTTDLFG | SEQ ID NO: 264 |
| | | | | | R | |
| 266 | hnRNPF | NP_004957.1 | RNA binding protein | Y266 | DLSyCLSGMYDHR | SEQ ID NO: 265 |
| 267 | hnRNPG | NP_002130.2 | RNA binding protein | Y206 | DVyLSPRDDGYSTKDSYSSR | SEQ ID NO: 266 |
| 268 | hnRNPG | NP_002130.2 | RNA binding protein | Y313 | YDDySSSRDGYGGSR | SEQ ID NO: 267 |
| 269 | hnRNPH | NP_005511.1 | RNA binding protein | Y243 | GAYGGGYGGyDDYNGYNDGYGFGSDRFGR | SEQ ID NO: 268 |
| 270 | hnRNPH | NP_005511.1 | RNA binding protein | Y249 | GAYGGGYGGYDDYNGyNDGYGFGSDRFGR | SEQ ID NO: 269 |
| 271 | hnRNPH | NP_062543.1 | RNA binding protein | Y253 | GAYGGGYGGYDDYGGYNDGyGFGSDRFGR | SEQ ID NO: 270 |
| 272 | hnRNPR | NP_005817.1 | RNA binding protein | Y431 | STAyEDYYYHPPPR | SEQ ID NO: 271 |
| 273 | hnRNPU | NP_004492.2 | RNA binding protein | Y801 | NQSQGYNQWQQGQFWGQKPWSQHyHQGYY | SEQ ID NO: 272 |
| 274 | hnRNPU | NP_004492.2 | RNA binding protein | Y805 | NQSQGYNQWQQGQFWGQKPWSQHYHQGYy | SEQ ID NO: 273 |
| 275 | hnRNP- | NP_004491.2 | RNA binding protein | Y126 | MYSyPARVPPPPPIAR | SEQ ID NO: 274 |
| | C1/C2 | | | | | |
| 276 | hnRNP-K | NP_002131.2 | RNA binding protein | Y280 | RDyDDMSPR | SEQ ID NO: 275 |
| 277 | hnRNP-K | NP_002131.2 | RNA binding protein | Y323 | GGDLMAyDRR | SEQ ID NO: 276 |
| 278 | hnRNP-K | NP_002131.2 | RNA binding protein | Y449 | IITITGTQDQIQNAQyLLQNSVK | SEQ ID NO: 277 |
| 279 | IGF2BP2 | NP_006539.3 | RNA binding protein | Y40 | SGyQFVDYPDQNWAIR | SEQ ID NO: 278 |
| 280 | KHSRP | NP_003676.1 | RNA binding protein | Y674 | QAQVATGGGPGQAPPGSQPDySAAWAEYYR | SEQ ID NO: 279 |
| 281 | KIAA0332 | NP_001073884.1 | RNA binding protein | Y634 | LCQIFSDLNATyR | SEQ ID NO: 280 |
| 282 | matrin 3 | NP_061322.2 | RNA binding protein | Y213 | SQESGyTDRMDYEDDRLR | SEQ ID NO: 281 |
| 283 | matrin 3 | NP_061322.2 | RNA binding protein | Y214 | SQESGYyDRMDYEDDRLR | SEQ ID NO: 282 |
| 284 | matrin 3 | NP_061322.2 | RNA binding protein | Y827 | NTHCSSLPHyQK | SEQ ID NO: 283 |
| 285 | MORC3 | NP_056173.1 | RNA binding protein | Y434 | KLPDGMDQLPEKWyCSNNPDPQFR | SEQ ID NO: 284 |
| 286 | IK | NP_006074.2 | Secreted protein | Y114 | DGVKDyEETELISTTANYR | SEQ ID NO: 285 |
| 287 | MIF | NP_002406.1 | Secreted protein | Y96 | ISPDRVyINYYDMNAANVGWNNSTFA | SEQ ID NO: 286 |
| 288 | MIF | NP_002406.1 | Secreted protein | Y99 | ISPDRVYINyYDMNAANVGWNNSTFA | SEQ ID NO: 287 |
| 289 | FOXL2 | NP_075555.1 | Transcriptional | Y127 | KGNyWTLDPACEDMFEKGNYR | SEQ ID NO: 288 |
| | | | regulator | | | |
| 290 | FOXL2 | NP_075555.1 | Transcriptional | Y258 | GLAGPAASYGPyTR | SEQ ID NO: 289 |
| | | | regulator | | | |
| 291 | GATA-1 | NP_002040.1 | Transcriptional | Y223 | DRTGHyLCNACGLYHK | SEQ ID NO: 290 |
| | | | regulator | | | |
| 292 | GATA-1 | NP_002040.1 | Transcriptional | Y285 | NASGDPVCNACGLyYK | SEQ ID NO: 291 |
| | | | regulator | | | |
| 293 | GATA-1 | NP_002040.1 | Transcriptional | Y78 | HSPVFQVyPLLNCMEGIPGGSPYAGWAYGK | SEQ ID NO: 292 |
| | | | regulator | | | |
| 294 | GATA4 | NP_002043.2 | Transcriptional | Y236 | DGTGHyLCANACGLYHK | SEQ ID NO: 293 |
| | | | regulator | | | |
| 295 | GATA4 | NP_002043.2 | Transcriptional | Y244 | DGTGHYLCNACGLyHK | SEQ ID NO: 294 |
| | | | regulator | | | |
| 296 | IFI-16 | NP_005522.2 | Transcriptional | Y648 | GEFTyYEIQDNTGK | SEQ ID NO: 295 |
| | | | regulator | | | |
| 297 | Ikaros | NP_006051.1 | Transcriptional | Y293 | GLSDTPyDSSASYEK | SEQ ID NO: 296 |
| | | | regulator | | | |
| 298 | Max | NP_002373.3 | Transcriptional | Y115 | SSAQLQTNyPSSDNSLYTNAK | SEQ ID NO: 297 |
| | | | regulator | | | |
| 299 | Max | NP_002373.3 | Transcriptional | Y70 | AQILDKATEyIQYMR | SEQ ID NO: 298 |
| | | | regulator | | | |
| 300 | MBD1 | NP_056670.2 | Transcriptional | Y380 | SWQCLQFAMKRLLPSVWSESEDGAGSPPPyR | SEQ ID NO: 299 |
| | | | regulator | | | |
| 301 | MDS1 | NP_004982.1 | Transcriptional | Y122 | FGPYVGEQRSNLKDPSyGWEVHLPR | SEQ ID NO: 300 |
| | | | regulator | | | |
| 302 | MLL2 | NP_003473.2 | Transcriptional | Y389 | FSPPEPGDTPTDEPDALyVACQGQPK | SEQ ID NO: 301 |
| | | | regulator | | | |
| 303 | MTA1 | NP_004680.2 | Transcriptional | Y11 | VGDyVYFENSSSNPYLIR | SEQ ID NO: 302 |
| | | | regulator | | | |
| 304 | MTA1 | NP_004680.2 | Transcriptional | Y13 | VGDYVyFENSSSNPYLIR | SEQ ID NO: 303 |
| | | | regulator | | | |
| 305 | MTA2 | NP_004730.2 | Transcriptional | Y11 | VGDyVYFENSSSNPYLVR | SEQ ID NO: 304 |
| | | | regulator | | | |
| 306 | IMP-1 | NP_006537.3 | Transcriptional | Y39 | SGyAFVDCPDEHWAMK | SEQ ID NO: 305 |
| | | | regulator | | | |
| 307 | LIMD1 | NP_055055.1 | Tumor suppressor | Y21 | DIELDMNyEASK | SEQ ID NO: 305 |
| 308 | MIB1 | NP_065825.1 | Ubiquitin conjugating | Y106 | CAECTNyDLCTVCYHGDKHHLR | SEQ ID NO: 307 |
| | | | system | | | |
| 309 | MIB1 | NP_065825.1 | Ubiquitin conjugating | Y194 | VTEIQDWASASSPHSAAyVLWDNGAK | SEQ ID NO: 308 |
| | | | system | | | |
| 310 | RFFL | NP_476519.1 | Ubiquitin conjugating | Y29 | MQAySNPGYSSFPSPTGLEPSCK | SEQ ID NO: 309 |
| | | | system | | | |
| 311 | RFFL | NP_476519.1 | Ubiquitin conjugating | Y34 | MQAYSNPGySSFPSPTGLEPSCK | SEQ ID NO: 310 |
| | | | system | | | |
| 312 | FLJ12525 | NP_112483.1 | Unknown function | Y536 | SPYTDLSLyWSVKPASSSFGSEAK | SEQ ID NO: 311 |
| 313 | FLJ13231 | NP_075561.2 | Unknown function | Y441 | DDDEYIKFLDLFLSyILERDLPYSR | SEQ ID NO: 312 |
| 314 | FLJ13231 | NP_075561.2 | Unknown function | Y449 | DDDEYIKFLDLFLSYILERDLPySR | SEQ ID NO: 313 |
| 315 | FLJ14732 | NP_115734.1 | Unknown function | Y439 | QKLKDLEQMLyKATVNAR | SEQ ID NO: 314 |
| 316 | FLJ20272 | NP_060205.3 | Unknown function | Y715 | LyAHVYGNGQSEKPDENEK | SEQ ID NO: 315 |
| 317 | FLJ20294 | NP_060219.2 | Unknown function | Y724 | RFLLPEyPLYAGIFHER | SEQ ID NO: 316 |
| 318 | FLJ20625 | NP_060377.1 | Unknown function | Y140 | IAAYAySALSQIR | SEQ ID NO: 317 |
| 319 | FLJ20643 | NP_060386.1 | Unknown function | Y194 | IQELGDLyTPAPGR | SEQ ID NO: 318 |
| 320 | FLJ21128 | NP_079359.2 | Unknown function | Y225 | AAVFEEDITyERR | SEQ ID NO: 319 |
| 321 | FLJ21128 | NP_079359.2 | Unknown function | Y258 | YRHDENILESEPIVyRR | SEQ ID NO: 320 |
| 322 | FLJ21783 | NP_076412.3 | Unknown function | Y544 | DEEDEDESyQSALANK | SEQ ID NO: 321 |
| 323 | FLJ21908 | NP_078880.1 | Unknown function | Y88 | IKSyDYEAWAK | SEQ ID NO: 322 |
| 324 | FLJ21908 | NP_078880.1 | Unknown function | Y90 | IKSYDyEAWAK | SEQ ID NO: 323 |
| 325 | FLJ22246 | NP_079508.2 | Unknown function | Y167 | SISDAPAPAyHDPLYEDQVSHR | SEQ ID NO: 324 |
| 326 | FLJ22246 | NP_079508.2 | Unknown function | Y172 | SISDAPAPAYHDPLyLEDQVSHR | SEQ ID NO: 325 |
| 327 | FLJ22662 | NP_079105.4 | Unknown function | Y452 | yIMRYNNYK | SEQ ID NO: 326 |
| 328 | FLJ22662 | NP_079105.4 | Unknown function | Y456 | YIMRyNNYK | SEQ ID NO: 327 |
| 329 | FLJ22794 | NP_071357.2 | Unknown function | Y24 | IEHyFSPVSK | SEQ ID NO: 328 |
| 330 | FLJ32786 | NP_653249.1 | Unknown function | Y529 | YSYFEPRyILVVPMNKEKYEGYLRR | SEQ ID NO: 329 |
| 331 | FLJ32786 | NP_653249.1 | Unknown function | Y540 | YSYFEPRYILVVPMNKEKyEGYLRR | SEQ ID NO: 330 |
| 332 | FLJ32786 | NP_653249.1 | Unknown function | Y543 | YSYFEPRYILVVPMNKEKYEGyLRR | SEQ ID NO: 331 |
| 333 | FLJ34931 | NP_001025054.1 | Unknown function | Y273 | REPQEQPNLLQQLLQyTVSK | SEQ ID NO: 332 |
| 334 | FLJ35894 | XP_001130007.1 | Unknown function | Y235 | PICSFIGLTLANNTyVQK | SEQ ID NO: 333 |
| 335 | FLJ36874 | NP_689929.1 | Unknown function | Y330 | LEHAyKPVQFEGSLGK | SEQ ID NO: 334 |
| 336 | FLJ36874 | NP_689929.1 | Unknown function | Y423 | ICSMyDNLRGK | SEQ ID NO: 335 |
| 337 | FLJ37562 | NP_689622.2 | Unknown function | Y35 | AADQFDIySSQQSK | SEQ ID NO: 336 |
| 338 | FLJ37874 | NP_872409.2 | Unknown function | Y328 | yDIDDENEIDENDVK | SEQ ID NO: 337 |
| 339 | FLJ39829 | NP_612450.2 | Unknown function | Y248 | SNTRAyY | SEQ ID NO: 338 |
| 340 | FLJ46867 | EAW84258.1 | Unknown function | Y521 | KLyECKECGKSYYSSGSFLNHKR | SEQ ID NO: 339 |
| 341 | FLJ46867 | EAW84258.1 | Unknown function | Y530 | KLYECKECGKSyYSSGSFLNHKR | SEQ ID NO: 340 |
| 342 | FLJ46867 | EAW84258.1 | Unknown function | Y531 | KLYECKECGKSYySSGSFLNHKR | SEQ ID NO: 341 |
| 343 | FNPB1 | NP_055848.1 | Unknown function | Y500 | RQSGLyDSQNPPTVNNCAQDR | SEQ ID NO: 342 |
| 344 | FOP | NP_008976.1 | Unknown function | Y337 | LISDKIGSLGLGTGEDDDyVDDFNSTSHR | SEQ ID NO: 343 |
| 345 | galactin- | NP_002299.2 | Unknown function | Y71 | FEDGGyVVCNTR | SEQ ID NO: 344 |
| | 9 | | | | | |
| 346 | GCET2 | NP_689998.1 | Unknown function | Y106 | VLCTRPSGNSAEEyYENVPCKAERPR | SEQ ID NO: 345 |
| 347 | GCET2 | NP_689998.1 | Unknown function | Y128 | ESLGGTETEySLLHMPSTDPR | SEQ ID NO: 346 |
| 348 | GCET2 | NP_689998.1 | Unknown function | Y148 | SPEDEyELLMPHR | SEQ ID NO: 347 |
| 349 | GCET2 | NP_689998.1 | Unknown function | Y80 | MSSTPIQDNVDQTySEELCYTLINHR | SEQ ID NO: 348 |
| 350 | GCET2 | NP_689998.1 | Unknown function | Y86 | MSSTPIQDNVDQTYSEELCyTLINHR | SEQ ID NO: 349 |
| 351 | GDAP2 | NP_060156.1 | Unknown function | Y222 | VVFAVSDLEEGTYQKLLPLyFPRSLK | SEQ ID NO: 350 |
| 352 | GIMAP7 | NP_694968.1 | Unknown function | Y206 | MVQCNEGAYFSDDIyKDTEER | SEQ ID NO: 351 |
| 353 | GLT25D2 | NP_055916.1 | Unknown function | Y467 | LMDNIDQAQLDWELIyIGR | SEQ ID NO: 352 |
| 354 | gm117 | NP_932343.1 | Unknown function | Y73 | SVTRPAFLyNPLNK | SEQ ID NO: 353 |
| 355 | GPR178 | XP_376550.4 | Unknown function | Y632 | NALyESQLK | SEQ ID NO: 354 |
| 356 | HBS1 | NP_006611.1 | Unknown function | Y513 | IEAGyIQTGDR | SEQ ID NO: 355 |
| 357 | HBS1 | NP_006611.1 | Unknown function | Y56 | DKPSVEPVEEyDYEDLKESSNSVSNHQLSGF | SEQ ID NO: 356 |
| | | | | | DQAR | |
| 358 | HBS1 | NP_006611.1 | Unknown function | Y58 | DKPSVEPVEEYDyEDLKESSNSVSNHQLSGF | SEQ ID NO: 357 |
| | | | | | DQAR | |
| 359 | HCA59 | NP_057604.1 | Unknown function | Y228 | FyHEELNAPIR | SEQ ID NO: 358 |
| 360 | HCA59 | NP_057604.1 | Unknown function | Y279 | ATDDYHyEKFK | SEQ ID NO: 359 |
| 361 | HMG20A | NP_060670.1 | Unknown function | Y152 | yLDEADRDKERYMKELEQYQK | SEQ ID NO: 360 |
| 362 | HMG20A | NP_060670.1 | Unknown function | Y163 | YLDEADRDKERyMKELEQYQK | SEQ ID NO: 361 |
| 363 | HMG20A | NP_060670.1 | Unknown function | Y170 | YLDEADRDKERYMKELEQyQK | SEQ ID NO: 362 |
| 364 | HNRPLL | NP_612403.2 | Unknown function | Y530 | VPNGSNPyTLK | SEQ ID NO: 363 |
| 365 | HSHIN1 | NP_955356.1 | Unknown function | Y1021 | SRDEGYQyHR | SEQ ID NO: 364 |
| 366 | HT008 | NP_060939.3 | Unknown function | Y299 | LSEVIyEPFQLLSK | SEQ ID NO: 365 |
| 367 | HT008 | NP_060939.3 | Unknown function | Y76 | EDLyLEPQVGHDPAGPAASPVLADGLSVSQA | SEQ ID NO: 366 |
| | | | | | PAILPVSK | |
| 368 | HYLS1 | NP_659451.1 | Unknown function | Y51 | EAQSIQYDPySK | SEQ ID NO: 367 |
| 369 | IFIT3 | NP_001540.2 | Unknown function | Y208 | QAIELSPDNQyVK | SEQ ID NO: 368 |
| 370 | IFIT5 | NP_036552.1 | Unknown function | Y216 | AVTLNPDNSyIK | SEQ ID NO: 369 |
| 371 | IFT88 | NP_006522.2 | Unknown function | Y243 | MNMGNIyLKQR | SEQ ID NO: 370 |
| 372 | IFT88 | NP_006522.2 | Unknown function | Y670 | SGNyQKALDTYKDTHR | SEQ ID NO: 371 |
| 373 | IFT88 | NP_006522.2 | Unknown function | Y677 | SGNYQKALDTyKDTHR | SEQ ID NO: 372 |
| 374 | IQSEC1 | NP_055684.3 | Unknown function | Y465 | SESDySDGDNDSINSTSNSNDTINCSSESSS | SEQ ID NO: 373 |
| | | | | | R | |
| 375 | KBTBD8 | NP_115894.1 | Unknown function | Y498 | KKDFPCDQSINPyLK | SEQ ID NO: 374 |
| 376 | KCTD12 | NP_612453.1 | Unknown function | Y119 | EAEyFELPELVRR | SEQ ID NO: 375 |
| 377 | KIAA0020 | NP_055693.4 | Unknown function | Y259 | KMLRHAEASAIVEYAyNDK | SEQ ID NO: 376 |
| 378 | KIAA0084 | NP_055965.1 | Unknown function | Y36 | IDVSyEYR | SEQ ID NO: 377 |
| 379 | KIAA0084 | NP_055965.1 | Unknown function | Y122 | TDLHNEGyILELDCCSSLDHPTDQK | SEQ ID NO: 378 |
| 380 | KIAA0157 | NP_115558.2 | Unknown function | Y106 | AIyQVYNALQEK | SEQ ID NO: 379 |
| 381 | KIAA0157 | NP_115558.2 | Unknown function | Y204 | STLGDAEASDPPPPySDFHPNNQESTLSHSR | SEQ ID NO: 380 |
| 382 | KIAA0157 | NP_115558.2 | Unknown function | Y238 | SVFMPRPQAVGSSNyASTSAGLKYPGSGADL | SEQ ID NO: 381 |
| | | | | | PPPQR | |
| 383 | KIAA0174 | NP_055576.2 | Unknown function | Y43 | EIADyLAAGKDER | SEQ ID NO: 382 |
| 384 | KIAA0182 | NP_055430.1 | Unknown function | Y724 | APDPAyIYDEFLQQR | SEQ ID NO: 383 |
| 385 | KIAA0258 | NP_001073965.1 | Unknown function | Y174 | GQSVKyVYK | SEQ ID NO: 384 |
| 386 | KIAA0280 | NP_055974.1 | Unknown function | Y85 | TyQASSAAFR | SEQ ID NO: 385 |
| 387 | KIAA0310 | NP_055681.1 | Unknown function | Y1390 | SHNVAAGSyEAPLPPGSFHGDFAYGTYR | SEQ ID NO: 386 |
| 388 | KIAA0310 | NP_055681.1 | Unknown function | Y1405 | SHNVAAGSYEAPLPPGSFHGDFAyGTYR | SEQ ID NO: 387 |
| 389 | KIAA0310 | NP_055681.1 | Unknown function | Y489 | yGPLPGPAVPR | SEQ ID NO: 388 |
| 390 | KIAA0310 | NP_055681.1 | Unknown function | Y525 | HGAVCHTGAPDATLHTVHPDSVSSSySSR | SEQ ID NO: 389 |
| 391 | KIAA0310 | NP_055681.1 | Unknown function | Y584 | QIDSSPVGGETDETTVSQNyR | SEQ ID NO: 390 |
| 392 | KIAA0310 | NP_055681.1 | Unknown function | Y991 | ANHSSHQEDTyGALDFTLSR | SEQ ID NO: 391 |
| 393 | KIAA0310 | NP_055681.1 | Unknown function | Y1009 | TLENPVNVyNPSHSDSLASQQSVASHPR | SEQ ID NO: 392 |
| 394 | KIAA0310 | NP_055681.1 | Unknown function | Y1041 | FyQQVTK | SEQ ID NO: 393 |
| 395 | KIAA0310 | NP_055681.1 | Unknown function | Y1199 | yRPYDGAASAYAQNYR | SEQ ID NO: 394 |
| 396 | KIAA0310 | NP_055681.1 | Unknown function | Y1202 | YRPyDGAASAYAQNYR | SEQ ID NO: 395 |
| 397 | KIAA0310 | NP_055681.1 | Unknown function | Y1209 | YRPYDGAASAyAQNYR | SEQ ID NO: 396 |
| 398 | KIAA0323 | NP_056114.1 | Unknown function | Y456 | HIVIDGSNVAMVHGLQHyFSSR | SEQ ID NO: 397 |
| 399 | KIAA0326 | NP_001073886.1 | Unknown function | Y346 | THTGEKPyECLECGK | SEQ ID NO: 398 |
| 400 | KIAA0329 | NP_055659.1 | Unknown function | Y37 | NAIPTKIQKGFRSIVVyLTALDTNGDYIAVG | SEQ ID NO: 399 |
| | | | | | SSIGML | |
| 401 | KIAA0372 | NP_055454.1 | Unknown function | Y1169 | CLLTSAIyALQGR | SEQ ID NO: 400 |
| 402 | KIAA0391 | NP_055487.2 | Unknown function | Y175 | NNGIVSyDLLVK | SEQ ID NO: 401 |
| 403 | KIAA0460 | NP_056018.1 | Unknown function | Y241 | NGPSLTEALENAGIFyEAQYKEVK | SEQ ID NO: 402 |
| 404 | KIAA0460 | NP_056018.1 | Unknown function | Y245 | NGPSLTEALENAGIFYEAQyKEVKVVANAYK | SEQ ID NO: 403 |
| | | | | | TFANR | |
| 405 | KIAA0467 | NP_056099.2 | Unknown function | Y1228 | SQEPIySEEASGPR | SEQ ID NO: 404 |
| 406 | KIAA0515 | NP_037450.2 | Unknown function | Y641 | QQQQQQQEQLyK | SEQ ID NO: 405 |
| 407 | KIAA0676 | NP_055858.2 | Unknown function | Y853 | RDPSLPyLEQYR | SEQ ID NO: 406 |
| 408 | KIAA0692 | XP_931084.2 | Unknown function | Y199 | AGATASKEPPLyYGVCPVYEDVPAR | SEQ ID NO: 407 |
| 409 | KIAA0692 | XP_931084.2 | Unknown function | Y200 | AGATASKEPPLYyGVCPVYEDVPAR | SEQ ID NO: 408 |
| 410 | KIAA0692 | XP_931084.2 | Unknown function | Y206 | AGATASKEPPLYYGVCPVyEDVPAR | SEQ ID NO: 409 |
| 411 | KIAA0748 | XP_374983.3 | Unknown function | Y364 | KLPTSPyPCVFCCEEETQQR | SEQ ID NO: 410 |
| 412 | KIAA0804 | NP_056118.2 | Unknown function | Y1255 | QDyCSICLQQYKR | SEQ ID NO: 411 |
| 413 | KIAA0853 | NP_055885.3 | Unknown function | Y592 | GSQIDSHSSNSNyHDSWETR | SEQ ID NO: 412 |
| 414 | KIAA0889 | AAI13406.1 | Unknown function | Y416 | AKPEPPKyGIVQEFFR | SEQ ID NO: 413 |
| 415 | KIAA1064 | NP_055983.1 | Unknown function | Y364 | GGMNDDEDFyDEDMGDGGGGSYR | SEQ ID NO: 414 |
| 416 | KIAA1161 | NP_065753.1 | Unknown function | Y295 | yMVRRYFNKPSR | SEQ ID NO: 415 |
| 417 | KIAA1161 | NP_065753.1 | Unknown function | Y300 | YMVRRyFNKPSR | SEQ ID NO: 416 |
| 418 | KIAA1161 | NP_065753.1 | Unknown function | Y685 | WRSyKGELFDKTPVLLTDYPVDLDEIAYFTW | SEQ ID NO: 417 |
| | | | | | AS | |
| 419 | KIAA1161 | NP_065753.1 | Unknown function | Y700 | WRSYKGELFDKTPVLLTDyPVDLDEIAYFTW | SEQ ID NO: 418 |
| | | | | | AS | |
| 420 | KIAA1228 | NP_065779.1 | Unknown function | Y796 | NLIAFSEDGSDPyVR | SEQ ID NO: 419 |
| 421 | KIAA1407 | NP_065868.1 | Unknown function | Y744 | NQQLEAIAKEHyER | SEQ ID NO: 420 |
| 422 | KIAA1458 | XP_044434.4 | Unknown function | Y778 | TYGSMKDDSWKDGCy | SEQ ID NO: 421 |
| 423 | KIAA1521 | NP_056450.2 | Unknown function | Y460 | SSSLEMTPyNTPQLSPATTPANKK | SEQ ID NO: 422 |
| 424 | KIAA1521 | NP_056450.2 | Unknown function | Y1080 | TSPSDGAMANyESTEVMGDGESAHDSPR | SEQ ID NO: 423 |
| 425 | KIAA1636 | XP_371074.3 | Unknown function | Y1165 | PDIMIILLSKLMEEGDMFyK | SEQ ID NO: 424 |
| 426 | KIAA1636 | XP_371074.3 | Unknown function | Y1615 | PSSAyRGGVRYSQTPQIGR | SEQ ID NO: 425 |
| 427 | KIAA1636 | XP_371074.3 | Unknown function | Y1635 | SQSASyYPVCHSK | SEQ ID NO: 426 |
| 428 | KIAA1636 | XP_371074.3 | Unknown function | Y1829 | TNNAQNGHLLEDDyYSPHGMLANGSR | SEQ ID NO: 427 |
| 429 | KIAA1636 | XP_371074.3 | Unknown function | Y1830 | TNNAQNGHLLEDDYySPHGMLANGSR | SEQ ID NO: 428 |
| 430 | KIAA1838 | NP_115824.1 | Unknown function | Y464 | QEVPMyTGPESR | SEQ ID NO: 429 |
| 431 | KIAA1838 | NP_115824.1 | Unknown function | Y764 | STSQLVNLQPDyINPR | SEQ ID NO: 430 |
| 432 | KIAA1913 | NP_443145.1 | Unknown function | Y164 | DIySTVIDIHTLR | SEQ ID NO: 431 |
| 433 | KIAA1984 | NP_078994.2 | Unknown function | Y683 | TAADELEAFLGGGAPGGRHPGGGDyEEL | SEQ ID NO: 432 |
| 434 | KIRREL | NP_060710.2 | Unknown function | Y432 | CDTIDTREEyEMKDPTNGYYNVR | SEQ ID NO: 433 |
| 435 | KIRREL | NP_060710.2 | Unknown function | Y441 | CDTIDTREEYEMKDPTNGyYNVR | SEQ ID NO: 434 |
| 436 | KIRREL | NP_060710.2 | Unknown function | Y442 | CDTIDTREEYEMKDPTNGYyNVR | SEQ ID NO: 435 |
| 437 | KIRREL | NP_060710.2 | Unknown function | Y566 | TPYEAYDPIGKyATATR | SEQ ID NO: 436 |
| 438 | KLHL11 | NP_060613.1 | Unknown function | Y121 | SVLAAATEyFTPLLSGQFSESR | SEQ ID NO: 437 |
| 439 | KNSL8 | NP_958929.1 | Unknown function | Y449 | HHEGGTPYAEyGGWYK | SEQ ID NO: 438 |
| 440 | LARP4 | NP_443111.3 | Unknown function | Y441 | ETSTLQVEQNGDyGR | SEQ ID NO: 439 |
| 441 | LARP4 | NP_443111.3 | Unknown function | Y72 | EYEVMySSSCETTR | SEQ ID NO: 440 |
| 442 | LDHAL6B | NP_149972.1 | Unknown function | Y194 | LIIVSNPVDILTyVAWK | SEQ ID NO: 441 |
| 443 | LEMD2 | NP_851853.1 | Unknown function | Y104 | AEPWLSQPASGSAYSTPGAyGDIRPSAASWV | SEQ ID NO: 442 |
| | | | | | GSR | |
| 444 | LEMD2 | NP_851853.1 | Unknown function | Y98 | AEPWLSQPASGSAyATPGAYGDIRPSAASWV | SEQ ID NO: 443 |
| | | | | | GSR | |
| 445 | LIME1 | NP_060276.1 | Unknown function | Y167 | ALPAAAATAGCAGLEATYSNVGLAALPGVSL | SEQ ID NO: 444 |
| | | | | | AASPVVAEyAR | |
| 446 | LIME1 | NP_060276.1 | Unknown function | Y200 | SPQEPQQGKTEVTPAAQVDVLySR | SEQ ID NO: 445 |
| 447 | LIN9 | NP_775106.2 | Unknown function | Y392 | LKSySMPISIEFQR | SEQ ID NO: 446 |
| 448 | LISCH | NP_057009.3 | Unknown function | Y290 | CPCCPDKCCCPEALyAAGK | SEQ ID NO: 447 |
| 449 | LISCH | NP_057009.3 | Unknown function | Y304 | AATSGVPSIyAPSTYAHLSPAK | SEQ ID NO: 448 |
| 450 | LISCH | NP_057009.3 | Unknown function | Y309 | AATSGVPSIYAPSTyAHLSPAK | SEQ ID NO: 449 |
| 451 | LLGL1 | NP_004131.3 | Unknown function | Y509 | VGCFDPySDDPR | SEQ ID NO: 450 |
| 452 | LMO7 | NP_005349.3 | Unknown function | Y1096 | STTELDDySTNK | SEQ ID NO: 451 |
| 453 | LMO7 | NP_005349.3 | Unknown function | Y363 | LFQKIyGENGSK | SEQ ID NO: 452 |
| 454 | LOC124245 | NP_653205.2 | Unknown function | Y385 | GGQyENFR | SEQ ID NO: 453 |
| 455 | LOC124245 | NP_653205.2 | Unknown function | Y392 | VQyTETEPYHNYR | SEQ ID NO: 454 |
| 456 | LOC124245 | NP_653205.2 | Unknown function | Y398 | VQYTETEPyHNYR | SEQ ID NO: 455 |
| 457 | LOC126295 | NP_775751.1 | Unknown function | Y61 | ANGSVSLQDMyGQEK | SEQ ID NO: 456 |
| 458 | LOC144100 | NP_778228.2 | Unknown function | Y524 | DGTVWQLyEWQQR | SEQ ID NO: 457 |
| 459 | LOC144100 | NP_778228.2 | Unknown function | Y412 | NGMLPASYGPGEQNGTGGyQR | SEQ ID NO: 458 |
| 460 | LOC144100 | NP_778228.2 | Unknown function | Y470 | QGPGQSLSFPENyQTLPK | SEQ ID NO: 459 |
| 461 | LOC148823 | NP_660321.1 | Unknown function | Y69 | SQEVSSTSNQENENGSGSEEVCyTVINHIPH | SEQ ID NO: 460 |
| | | | | | QR | |
| 462 | LOC148823 | NP_660321.1 | Unknown function | Y89 | SSLSSNDDGyENIDSLTR | SEQ ID NO: 461 |
| 463 | LOC253012 | NP_937794.1 | Unknown function | Y363 | yQPYKVIKQK | SEQ ID NO: 462 |
| 464 | LOC257106 | NP_859071.2 | Unknown function | Y816 | RTQTCTEGGDyCLIPR | SEQ ID NO: 463 |
| 465 | LOC440388 | EAW95596.1 | Unknown function | Y26 | yDIGGKYSHLPYNKYSVLLPLVAKEGK | SEQ ID NO: 466 |
| 466 | LOC440388 | EAW95596.1 | Unknown function | Y32 | YDIGGKySHLPYNKYSVLLPLVAKEGK | SEQ ID NO: 467 |
| 467 | LOC440388 | EAW95596.1 | Unknown function | Y37 | YDIGGKYSHLPyNKYSVLLPLVAKEGK | SEQ ID NO: 468 |
| 468 | LOC440388 | EAW95596.1 | Unknown function | Y40 | YDIGGKYSHLPYNKySVLLPLVAKEGK | SEQ ID NO: 469 |
| 469 | LOXHD1 | NP_653213.4 | Unknown function | Y1155 | ELVPyDIFTEKYMK | SEQ ID NO: 470 |
| 470 | LRBA | NP_006717.1 | Unknown function | Y2146 | yLLQNTALEIFMANR | SEQ ID NO: 471 |
| 471 | LRPPRC | NP_573566.2 | Unknown function | Y207 | LIASyCNVGDIEGASK | SEQ ID NO: 472 |
| 472 | LRPR1 | CAA65884.1 | Unknown function | Y637 | TKSEFNFSSKTyQEFNYYLTSMVGCLWTSK | SEQ ID NO: 473 |
| 473 | LRPR1 | CAA65884.1 | Unknown function | Y642 | TKSEFNFSSKTYQEFNyYLTSMVGCLWTSK | SEQ ID NO: 474 |
| 474 | LRPR1 | CAA65884.1 | Unknown function | Y643 | TKSEFNFSSKTYQEFNYyLTSMVGCLWTSK | SEQ ID NO: 475 |
| 475 | LRRFIP2 | NP_006300.1 | Unknown function | Y300 | SDKQyAENYTRPSSR | SEQ ID NO: 476 |
| 476 | LSR7 | NP_061029.2 | Unknown function | Y267 | LAEQVSSyNESK | SEQ ID NO: 477 |
| 477 | LUZP1 | NP_361013.2 | Unknown function | Y952 | NVESTNSNAyTQR | SEQ ID NO: 478 |
| 478 | MAGE-D2 | NP_055414.2 | Unknown function | Y439 | VPNSNPPEyEFFWGLR | SEQ ID NO: 479 |
| 479 | MAGOH | NP_002361.1 | Unknown function | Y34 | yANNSNYKNDVMIR | SEQ ID NO: 480 |
| 480 | Meg-3 | NP_073744.2 | Unknown function | Y482 | KYDyDSSSVR | SEQ ID NO: 481 |
| 481 | MGC23244 | NP_653216.1 | Unknown function | Y222 | GQSIySTSFPQPAPR | SEQ ID NO: 482 |
| 482 | MGC32065 | NP_695003.1 | Unknown function | Y264 | LTPTHAASPVyR | SEQ ID NO: 483 |
| 483 | MGC32065 | NP_695003.1 | Unknown function | Y70 | SGISTNHADySSSPAGSPGAQVSLYNSPSVA | SEQ ID NO: 484 |
| | | | | | SPAR | |
| 484 | MGC32065 | NP_695003.1 | Unknown function | Y85 | SGISTNHADYSSSPAGSPGAQVSLyNSPSVA | SEQ ID NO: 485 |
| | | | | | SPAR | |
| 485 | MGC33424 | NP_714916.2 | Unknown function | Y237 | VLLPDLEFyVNLGDWPLEHRK | SEQ ID NO: 486 |
| 486 | MGC41917 | NP_001034743.1 | Unknown function | Y218 | LIHTGEKPyKCL | SEQ ID NO: 487 |
| 487 | MGC48595 | NP_976054.1 | Unknown function | Y729 | DKMVLECLLNLMQRDPyWK | SEQ ID NO: 488 |
| 488 | MLF2 | NP_005430.1 | Unknown function | Y121 | VyQETSEMR | SEQ ID NO: 489 |
| 489 | MLL4 | NP_055542.1 | Unknown function | Y1275 | HAyHPACLGPSYPTRATRKR | SEQ ID NO: 490 |
| 490 | MLL4 | NP_055542.1 | Unknown function | Y1284 | HAYHPACLGPSyPTRATRKR | SEQ ID NO: 491 |
| 491 | MPHOSPH8 | NP_059990.2 | Unknown function | Y716 | QSNNVLVyDLLK | SEQ ID NO: 492 |
| 492 | GOLGA3 | NP_005886.2 | Vesicle protein | Y512 | NASLASSNNDLQVAEEQyQR | SEQ ID NO: 493 |
| 493 | GOLGB1 | NP_004478.2 | Vesicle protein | Y963 | QNyDEMSPAGQISK | SEQ ID NO: 494 |
| 494 | HEP-COP | NP_004362.1 | Vesicle protein | Y733 | DMSGHyQNALYLGDVESERVR | SEQ ID NO: 495 |
| 495 | LAPTM4A | NP_055528.1 | Vesicle protein | Y210 | NVPEIAVYPAFEAPPQyVLPTYEMAVK | SEQ ID NO: 496 |
| 496 | M6PRBP1 | NP_005808.2 | Vesicle protein | LEPQIASASEyAHR | SEQ ID NO: 497 | |

The short name for each protein in which a phosphorylation site has presently been identified is provided in Column A, and its SwissProt accession number (human) is provided Column B. The protein type/group into which each protein falls is provided in Column C. The identified tyrosine residue at which phosphorylation occurs in a given protein is identified in Column D, and the amino acid sequence of the phosphorylation site encompassing the tyrosine residue is provided in Column E (lower case y=the tyrosine (identified in Column D)) at which phosphorylation occurs. Table 1 above is identical to FIG. 2, except that the latter includes the disease and cell type(s) in which the particular phosphorylation site was identified (Columns F and G).

One of skill in the art will appreciate that, in many instances the utility of the instant invention is best understood in conjunction with an appreciation of the many biological roles and significance of the various target signaling proteins/polypeptides of the invention. The foregoing is illustrated in the following paragraphs summarizing the knowledge in the art relevant to a few non-limiting representative peptides containing selected phosphorylation sites according to the invention.

Galectin-9 (O00182), phosphorylated at Y71, is among the proteins listed in this patent. Galectin-9, Galectin 9, a urate transporter with eosinophil chemoattractant activity, induces apoptosis and dendritic cell maturation and antigen presentation, protein expression is upregulated in breast neoplasms and melanoma; mRNA is upregulated in Hodgkin disease. This protein has potential diagnostic and/or therapeutic implications based on the following findings. Increased expression of LGALS9 in lymphoid organs correlates with Hodgkin's disease (JBC 272: 6416-22 (1997)). Increased expression of LGALS9 in lymphoid organs correlates with Hodgkin's disease (J Biol Chem 272: 6416-22 (1997)). Decreased expression of LGALS9 protein correlates with increased incidence of disease progression associated with melanoma (Int J Cancer 99: 809-16 (2002)). (PhosphoSite®, Cell Signaling Technology (Danvers, Mass.), Human PSD™, Biobase Corporation, (Beverly, Mass.)).

Grb10 (Q13322), phosphorylated at Y404, is among the proteins listed in this patent. Grb10, Growth factor receptor-bound protein 10, an adaptor protein that binds to various receptor and cytosolic kinases and regulates glycogen biosynthesis; variants may be associated with Russell-Silver Syndrome. This protein has potential diagnostic and/or therapeutic implications based on the following findings. GRB10 map position correlates with growth disorders (Am J Hum Genet 68: 247-53 (2001)). Missense mutation in the GRB10 gene correlates with growth disorders (Am J Hum Genet 67: 476-82 (2000)). (PhosphoSite®, Cell Signaling Technology (Danvers, Mass.), Human PSD™, Biobase Corporation, (Beverly, Mass.)).

GSK3B (P49841), phosphorylated at Y71, is among the proteins listed in this patent. GSK3B, Glycogen synthase kinase 3 beta, serine/threonine kinase that regulates beta-catenin (CTNNB1) stability and binds presenilin 1 (PSEN1), associated with Alzheimer disease, bipolar disorder, schizophrenia and various neoplasms. This protein has potential diagnostic and/or therapeutic implications based on the following findings. Increased expression of GSK3B protein may correlate with hepatocellular carcinoma (Cancer Lett 199: 201-8 (2003)). Induced inhibition of GSK3B protein may prevent increased cell proliferation associated with prostatic neoplasms (Oncogene 23: 7882-92 (2004)). Increased phosphorylation of GSK3B may correlate with hepatocellular carcinoma associated with liver neoplasms (Cancer Lett 199: 201-8 (2003)). Decreased expression of GSK3B protein may prevent increased protein amino acid phosphorylation associated with Alzheimer disease (Proc Natl Acad Sci U S A 99: 1140-5 (2002)). Increased glycogen synthase kinase 3 activity of GSK3B may prevent increased cell proliferation associated with prostatic neoplasms (JBC 279: 32444-52 (2004)). Decreased expression of GSK3B protein may prevent increased protein amino acid phosphorylation associated with Alzheimer disease (PNAS 99: 1140-5 (2002)). Decreased expression of GSK3B protein may. correlate with increased cell differentiation associated with colonic neoplasms (Oncol Res 12: 193-201 (2000)). Increased phosphorylation of GSK3B may correlate with hepatocellular carcinoma (Cancer Lett 199: 201-8 (2003)). Decreased expression of GSK3B protein may prevent increased protein amino acid phosphorylation associated with Alzheimer disease (Proc Natl Acad Sci USA 99: 1140-5 (2002)). Increased glycogen synthase kinase 3 activity of GSK3B may prevent increased cell proliferation associated with prostatic neoplasms (J Biol Chem 279: 32444-52 (2004)). Increased expression of GSK3B protein may correlate with hepatocellular carcinoma associated with liver neoplasms (Cancer Lett 199: 201-8 (2003)). Decreased phosphorylation of GSK3B may correlate with anoxia (JBC 278: 31277-85 (2003)). Increased expression of GSK3B in brain correlates with Alzheimer disease (Genomics 60: 121-8 (1999)). Decreased phosphorylation of GSK3B may correlate with anoxia (J Biol Chem 278: 31277-85 (2003)). (PhosphoSite®, Cell Signaling Technology (Danvers, Mass.), Human PSD™, Biobase Corporation, (Beverly, Mass.)).

GSTP1 (P09211), phosphorylated at Y63, Y198, is among the proteins listed in this patent. GSTP1, Glutathione S-transferase pi, a member of the pi class of glutathione S-transferases, involved in carcinogen detoxification and protection against reactive oxygen species; alleles may be risk factor for Parkinson disease, multiple sclerosis, and cancers. This protein has potential diagnostic and/or therapeutic implications based on the following findings. Polymorphism in the GSTP1 gene correlates with adenocarcinoma tumors associated with esophageal neoplasms (Cancer Res 59: 586-9 (1999)). Decreased expression of GSTP1 protein correlates with carcinoma tumors associated with prostatic neoplasms (PNAS 91: 11733-7 (1994)). Decreased expression of GSTP1 in bronchi correlates with bronchogenic carcinoma (Cancer Res 60: 1609-18 (2000)). Missense mutation in the GSTP1 gene correlates with bladder neoplasms (Carcinogenesis 18: 641-4 (1997)). Increased expression of GSTP1 protein correlates with increased occurrence of disease progression associated with B-cell lymphoma (Leukemia 17: 972-7 (2003)). Polymorphism in the GSTP1 gene correlates with Barrett esophagus associated with esophageal neoplasms (Cancer Res 59: 586-9 (1999)). Missense mutation in the GSTP1 gene correlates with increased occurrence of more severe form of skin neoplasms (Pharmacogenetics 10: 545-56 (2000)). Increased expression of GSTP1 protein correlates with non-small-cell lung carcinoma associated with lung neoplasms (Cancer 73: 1377-82 (1994)). Polymorphism in the GSTP1 gene correlates with increased occurrence of familial form of prostatic neoplasms (Anticancer Res 23: 2897-902 (2003)). Increased expression of GSTP1 protein correlates with decreased cell proliferation associated with non-small-cell lung carcinoma (Cancer 70: 764-9 (1992)). Increased expression of GSTP1 protein correlates with decreased response to drug associated with hepatocellular carcinoma (Mol Carcinog 29: 170-8 (2000)). Increased expression of GSTP1 mRNA correlates with decreased response to drug associated with ovarian neoplasms (Anticancer Res 14: 193-200 (1994)). Increased expression of GSTP1 protein correlates with drug-induced form of lung neoplasms (Br J Cancer 64: 700-4 (1991)). Increased expression of GSTP1 protein may correlate with decreased response to drug associated with non-small-cell lung carcinoma (Cancer 73: 1377-82 (1994)). Increased expression of GSTP1 protein may correlate with increased occurrence of drug-resistant form of bone neoplasms (Cancer 79: 2336-44 (1997)). Increased expression of GSTP1 protein may correlate with osteosarcoma tumors associated with bone neoplasms (Cancer 79: 233644 (1997)). Increased expression of GSTP1 protein correlates with decreased response to drug associated with ovarian neoplasms (Cancer 79: 521-7 (1997)). Polymorphism in the GSTP1 gene correlates with decreased occurrence of genetic predisposition to disease associated with prostatic neoplasms (Int J Cancer 95: 152-5 (2001)). Hypermethylation of the GSTP1 promoter correlates with non-small-cell lung carcinoma associated with lung neoplasms (Cancer Res 61: 249-55 (2001)). Polymorphism in the GSTP1 gene correlates with increased response to chemical stimulus associated with asthma (Pharmacogenetics 11: 437-45 (2001)). Decreased expression of GSTP1 in epithelium/epithelial cells correlates with bronchogenic carcinoma (Cancer Res 60: 1609-18 (2000)). Increased expression of GSTP1 mRNA correlates with recurrence associated with acute myelocytic leukemia (Leukemia 10: 426-33 (1996)). Polymorphism in the GSTP1 gene may cause abnormal response to oxidative stress associated with breast neoplasms (Cancer Lett 151: 87-95 (2000)). Amplification of the GSTP1 gene correlates with drug-resistant form of squamous cell carcinoma (Cancer Res 63: 8097-102 (2003)). Hypermethylation of the GSTP1 promoter correlates with carcinoma tumors associated with breast neoplasms (Cancer Res 58: 4515-8 (1998)). Increased expression of GSTP1 protein correlates with decreased occurrence of death associated with ovarian neoplasms (Br J Cancer 68: 235-9 (1993)). Hypermethylation of the GSTP1 promoter may correlate with precancerous conditions associated with non-small-cell lung carcinoma (Cancer Res 61: 249-55 (2001)). Hypermethylation of the GSTP1 promoter correlates with carcinoma tumors associated with prostatic neoplasms (Proc Natl Acad Sci USA 91: 11733-7 (1994)). Hypermethylation of the GSTP1 promoter correlates with increased aflatoxin B I metabolic process associated with liver neoplasms (Cancer Lett 221: 13543 (2005)). Polymorphism in the GSTP1 gene correlates with acute lymphocytic leukemia (L1) (Pharmacogenetics 12: 655-8 (2002)). Decreased expression of GSTP1 protein correlates with carcinoma tumors associated with prostatic neoplasms (Proc Natl Acad Sci U S A 91: 11733-7 (1994)). Polymorphism in the GSTP1 gene correlates with increased occurrence of genetic predisposition to disease associated with prostatic neoplasms (Anticancer Res 23: 2897-902 (2003)). Increased expression of GSTP1 protein correlates with decreased severity of pathologic neovascularization associated with lung neoplasms (Carcinogenesis 16: 2129-33 (1995)). Decreased expression of GSTP1 protein may cause increased response to drug associated with hepatocellular carcinoma (J Biol Chem 277: 38954-64 (2002)). Polymorphism in the GSTP1 gene may cause increased occurrence of early onset form of prostatic neoplasms (Pharmacogenetics 11: 325-30 (2001)). Hypermethylation of the GSTP1 gene correlates with prostatic intraepithelial neoplasia associated with prostatic neoplasms (Int J Cancer 106: 382-7 (2003)). Polymorphism in the GSTP1 gene correlates with disease susceptibility associated with breast neoplasms (Int J Cancer 91: 334-9 (2001)). Missense mutation in the GSTP1 gene correlates with decreased occurrence of death associated with multiple myeloma (Blood 102: 2345-50 (2003)). Hypermethylation of the GSTP1 gene correlates with prostatic neoplasms (Cancer Lett 205: 181-8 (2004)). Lack of expression of GSTP1 protein correlates with drug-sensitive form of non-small-cell lung carcinoma (Cancer 78: 416-21 (1996)). Decreased glutathione transferase activity of GSTP1 may cause decreased response to toxin associated with lung neoplasms (Pharmacogenetics 11: 757-64 (2001)). Hypermethylation of the GSTP1 promoter correlates with early stage or low grade form of prostatic neoplasms (J Natl Cancer Inst 93: 1747-52 (2001)). Lack of expression of GSTP1 protein correlates with drug-sensitive form of lung neoplasms (Cancer 78: 416-21 (1996)). Polymorphism in the GSTP1 gene correlates with squamous cell carcinoma tumors associated with esophageal neoplasms (Int J Cancer 79: 517-20 (1998)). Increased expression of GSTP1 protein correlates with lung neoplasms (Carcinogenesis 16: 707-11 (1995)). Increased expression of GSTP1 protein correlates with decreased cell proliferation associated with lung neoplasms (Cancer 70: 764-9 (1992)). Hypermethylation of the GSTP1 promoter correlates with carcinoma tumors associated with prostatic neoplasms (Proc Natl Acad Sci U S A 91: 11733-7 (1994)). Polymorphism in the GSTP1 gene may cause decreased response to toxin associated with lung neoplasms (Pharmacogenetics 11: 757-64 (2001)). Polymorphism in the GSTP1 gene correlates with disease susceptibility associated with small cell carcinoma (Carcinogenesis 23: 1475-81 (2002)). Polymorphism in the GSTP1 gene correlates with decreased incidence of recurrence associated with acute lymphocytic leukemia (L1) (Blood 95: 1222-8 (2000)). Hypermethylation of the GSTP1 promoter correlates with carcinoma tumors associated with prostatic neoplasms (PNAS 91: 11733-7 (1994)). Increased expression of GSTP1 protein may correlate with decreased response to drug associated with lung neoplasms (Cancer 73: 1377-82 (1994)). Hypermethylation of the GSTP1 promoter correlates with non-familial form of breast neoplasms (Hum Mol Genet 10: 3001-3007 (2001)). Increased expression of GSTP1 mRNA correlates with esophageal neoplasms (Cancer 67: 2560-4 (1991)). Increased expression of GSTP1 protein correlates with increased occurrence of death associated with B-cell lymphoma (Leukemia 17: 972-7 (2003)). Hypermethylation of the GSTP1 promoter correlates with increased aflatoxin BI metabolic process associated with hepatocellular carcinoma (Cancer Lett 221: 135-43 (2005)). Increased expression of GSTP1 mRNA may prevent increased occurrence of Barrett esophagus associated with esophageal neoplasms (Mol Carcinog 24: 128-36 (1999)). Polymorphism in the GSTP1 gene may cause increased response to UV associated with squamous cell carcinoma (Kidney Int 58: 2186-93 (2000)). Decreased glutathione transferase activity of GSTP1 correlates with decreased occurrence of death associated with breast neoplasms (Cancer Res 60: 5621-4 (2000)). Polymorphism in the GSTP1 gene correlates with Hodgkin's disease (Hum Mol Genet 10: 1265-73 (2001)). Increased expression of GSTP1 protein correlates with drug-resistant form of non-small-cell lung carcinoma (Br J Cancer 64: 700-4 (1991)). Increased expression of GSTP1 protein may correlate with increased occurrence of local neoplasm recurrence associated with breast neoplasms (J Natl Cancer Inst 89: 639-45 (1997)). Polymorphism in the GSTP1 gene correlates with squamous cell carcinoma tumors associated with esophageal neoplasms (Int J Cancer 89: 458-64 (2000)). Increased expression of GSTP1 protein correlates with decreased response to drug associated with liver neoplasms (Mol Carcinog 29: 170-8 (2000)). Hypermethylation of the GSTP1 gene correlates with prostatic neoplasms (Cancer Res 64: 1975-86 (2004)). Single nucleotide polymorphism in the GSTP1 gene correlates with decreased occurrence of death associated with multiple myeloma (Blood 102: 2345-50 (2003)). Increased expression of GSTP1 mRNA may correlate with drug-resistant form of neuroblastoma (Int J Cancer 47: 732-7 (1991)). Hypermethylation of the GSTP1 promoter correlates with adenocarcinoma tumors associated with prostatic neoplasms (J Natl Cancer Inst 93: 1747-52 (2001)). Hypermethylation of the GSTP1 promoter may correlate with precancerous conditions associated with lung neoplasms (Cancer Res 61: 249-55 (2001)). Increased expression of GSTP1 protein correlates With decreased severity of pathologic neovascularization associated with non-small-cell lung carcinoma (Carcinogenesis 16: 2129-33 (1995)). Decreased expression of GSTP1 mRNA correlates with chronic lymphocytic leukemia (Leukemia 9: 1742-7 (1995)). Hypomethylation of the GSTP1 promoter may prevent prostatic neoplasms (Cancer Res 61: 8611-6 (2001)). Decreased glutathione transferase activity of GSTP1 may correlate with disease susceptibility associated with lung neoplasms (Cancer Lett 173: 155-62 (2001)). Hypermethylation of the GSTP1 promoter correlates with increased response to toxin associated with liver neoplasms (Cancer Lett 221: 135-43 (2005)). Polymorphism in the GSTP1 gene correlates with increased occurrence of central nervous system neoplasms associated with acute lymphocytic leukemia (Pharmacogenetics 10: 715-26 (2000)). Decreased expression of GSTP1 protein may cause increased response to drug associated with hepatocellular carcinoma (JBC 277: 38954-64 (2002)). Increased expression of GSTP1 protein correlates with drug-resistant form of lung neoplasms (Br J Cancer 64: 700-4 (1991)). Polymorphism in the GSTP1 gene correlates with decreased occurrence of lymphatic metastasis associated with breast neoplasms (Pharmacogenetics 8: 441-7 (1998)). Hypermethylation of the GSTP1 promoter correlates with carcinoma tumors associated with prostatic neoplasms (Cancer Res 60: 5941-5 (2000)). Hypermethylation of the GSTP1 promoter correlates with bladder neoplasms (Cancer Res 61: 8659-63 (2001)). Increased expression of GSTP1 protein correlates with drug-induced form of non-small-cell lung carcinoma (Br J Cancer 64: 700-4 (1991)). Polymorphism in the GSTP1 gene correlates with increased occurrence of small cell carcinoma associated with lung neoplasms (Carcinogenesis 23: 1475-81 (2002)). Decreased expression of GSTP1 protein correlates with carcinoma associated with cervix neoplasms (Anticancer Res 17: 4305-9 (1997)). Increased expression of GSTP1 protein correlates with non-small-cell lung carcinoma (Cancer 73: 1377-82 (1994)). Decreased glutathione transferase activity of GSTP1 may cause decreased response to toxin associated with squamous cell carcinoma (Pharmacogenetics 11: 757-64 (2001)). Polymorphism in the GSTP1 gene correlates with disease susceptibility associated with lung neoplasms (Carcinogenesis 23: 1475-81 (2002)). Polymorphism in the GSTP1 gene correlates with non-Hodgkin's lymphoma (Hum Mol Genet 10: 1265-73 (2001)). Decreased glutathione transferase activity of GSTP1 may cause Barrett esophagus associated with esophageal neoplasms (Cancer Res 59: 586-9 (1999)). Hypermethylation of the GSTP1 promoter correlates with increased response to toxin associated with hepatocellular carcinoma (Cancer Lett 221: 135-43 (2005)). Polymorphism in the GSTP1 gene correlates with disease susceptibility associated with lung neoplasms (Cancer Res 62: 2819-23 (2002)). Decreased glutathione transferase activity of GSTP1 may cause adenocarcinoma tumors associated with esophageal neoplasms (Cancer Res 59: 586-9 (1999)). Decreased expression of GSTP1 protein correlates with carcinoma tumors associated with prostatic neoplasms (Proc Natl Acad Sci USA 91: 11733-7 (1994)). Hypermethylation of the GSTP1 promoter correlates with adenocarcinoma tumors associated with prostatic neoplasms (J Natl Cancer Inst 95: 1634-7 (2003)). Polymorphism in the GSTP1 gene correlates with squamous cell carcinoma tumors associated with skin neoplasms (Kidney Int 58: 2186-93 (2000)). Hypermethylation of the GSTP1 promoter correlates with hepatocellular carcinoma associated with liver neoplasms (Cancer Lett 221: 135-43 (2005)). Hypermethylation of the GSTP1 promoter correlates with non-small-cell lung carcinoma associated with non-small-cell lung carcinoma (Cancer Res 61: 249-55 (2001)). Polymorphism in the GSTP1 gene may cause decreased response to toxin associated with squamous cell carcinoma (Pharmacogenetics 11: 757-64 (2001)). Increased expression of GSTP1 protein correlates with decreased response to drug associated with ovarian neoplasms (Br J Cancer 68: 235-9 (1993)). Hypermethylation of the GSTP1 promoter may correlate with hormone-dependent neoplasms associated with breast neoplasms (Gene 210: 1-7 (1998)). Polymorphism in the GSTP1 gene correlates with decreased occurrence of death associated with breast neoplasms (Cancer Res 60: 5621-4 (2000)). Decreased glutathione transferase activity of GSTP1 may correlate with increased response to drug associated with breast neoplasms (Cancer Res 60: 5621-4 (2000)). (PhosphoSite®, Cell Signaling Technology (Danvers, Mass.), Human PSD™, Biobase Corporation, (Beverly, Mass.)).

Hck (P08631), phosphorylated at Y329, is among the proteins listed in this patent. Hck, Hematopoietic cell kinase, a Src family tyrosine kinase involved in signaling, phagocytosis and cell shape changes in myeloid cell types, and in HIV-1 replication and spreading; mouse Hck mediates the development of encephalomyocarditis-induced diabetes. This protein has potential diagnostic and/or therapeutic implications based on the following findings. Viral exploitation of the protein-tyrosine kinase activity of HCK may cause increased macrophages survival associated with HIV infections (JBC 276: 25605-11 (2001)). Bacterial exploitation of the protein-tyrosine kinase activity of HCK may cause increased phagocytosis, engulfinent associated with Q fever (Infect Immun 69: 2520-6 (2001)). Decreased protein-tyrosine kinase activity of HCK may prevent increased cell proliferation associated with myeloid leukemia (J Biol Chem 275: 18581-5 (2000)). Bacterial exploitation of the protein-tyrosine kinase activity of HCK may cause increased actin filament organization associated with Q fever (Infect Immun 69: 2520-6 (2001)). Abnormal protein binding of HCK may cause increased cell proliferation associated with myeloid leukemia (J Biol Chem 275: 18581-5 (2000)). Viral exploitation of the protein-tyrosine kinase activity of HCK causes increased viral infectious cycle associated with HIV infections (JBC 276: 16885-93 (2001)). Decreased protein-tyrosine kinase activity of HCK may prevent increased cell proliferation associated with myeloid leukemia (JBC 275: 18581-5 (2000)). Viral exploitation of the protein-tyrosine kinase activity of HCK may cause increased macrophages survival associated with HIV infections (J Biol Chem 276: 25605-11 (2001)). Abnormal protein binding of HCK may cause increased cell proliferation associated with myeloid leukemia (JBC 275: 18581-5 (2000)). Viral exploitation of the protein-tyrosine kinase activity of HCK causes increased viral infectious cycle associated with HIV infections (J Biol Chem 276: 16885-93 (2001)). (PhosphoSite®, Cell Signaling Technology (Danvers, Mass.), Human PSD™, Biobase Corporation, (Beverly, Mass.)).

HDAC2 (Q92769), phosphorylated at Y88, is among the proteins listed in this patent. HDAC2, Histone deacetylase 2, mediates transcriptional repression of several transcriptional repressors by deacetylating histones, modulates repressor activity by YY1 deacetylation, acts in the inflammatory response; possible therapeutic target for colon cancer. This protein has potential diagnostic and/or therapeutic implications based on the following findings. (PhosphoSite®, Cell Signaling Technology (Danvers, Mass.), Human PSD™, Biobase Corporation, (Beverly, Mass.)).

HLAB (P01889), phosphorylated at Y344, is among the proteins listed in this patent. HLAB, Major histocompatibility complex class 1 B, an MHC heavy chain involved in the immune response, binds HIV peptide antigens; allelic variants are associated with Behcet Syndrome, multiple sclerosis, AIDS progression, and malaria resistance. This protein has potential diagnostic and/or therapeutic implications based on the following findings. Polymorphism in the HLA-B gene correlates with acquired immunodeficiency syndrome (J Virol 76: 12603-10 (2002)). Polymorphism in the HLA-B gene correlates with viremia associated with acquired immunodeficiency syndrome (Proc Natl Acad Sci USA 98: 5140-5 (2001)). Polymorphism in the HLA-B gene correlates with decreased occurrence of more severe form of Falciparum malaria (Nature 360: 434-9 (1992)). Abnormal expression of HLA-B in lymphocytes correlates with increased occurrence of death associated with skin neoplasms (Eur J Cancer 30: 294-8 (1994)). Polymorphism in the HLA-B gene correlates with viremia associated with acquired immunodeficiency syndrome (Proc Natl Acad Sci USA 98: 5140-5 (2001)). Decreased expression of HLA-B mRNA may correlate with esophageal neoplasms associated with squamous cell carcinoma (Carcinogenesis 22: 1615-23 (2001)). Polymorphism in the HLA-B gene correlates with disease susceptibility associated with acquired immunodeficiency syndrome (Proc Natl Acad Sci USA 98: 5140-5 (2001)). Polymorphism in the HLA-B gene may correlate with graft-vs-host disease associated with leukemia (Blood 99: 4200-6 (2002)). Decreased expression of HLA-B protein may cause decreased active T-cells function associated with leukemia (Blood 103: 3122-30 (2004)). Polymorphism in the HLA-B gene correlates with decreased occurrence of disease progression associated with HIV infections (Proc Natl Acad Sci USA 97: 2709-14 (2000)). Abnormal expression of HLA-B in lymphocytes correlates with increased severity of melanoma associated with skin neoplasms (Eur J Cancer 30: 294-8 (1994)). Hypermethylation of the HLA-B gene may correlate with esophageal neoplasms associated with squamous cell carcinoma (Carcinogenesis 22: 1615-23 (2001)). Decreased expression of HLA-B protein may correlate with non-small-cell lung carcinoma (Cancer Res 51: 2463-8 (1991)). Decreased expression of HLA-B protein may correlate with leukemia (Blood 103: 3122-30 (2004)). Polymorphism in the HLA-B gene correlates with disease susceptibility associated with acquired immunodeficiency syndrome (PNAS 98: 5140-5 (2001)). Abnormal expression of HLA-B in lymphocytes correlates with increased severity of disease progression associated with melanoma (Eur J Cancer 30: 294-8 (1994)). Polymorphism in the HLA-B gene correlates with viremia associated with acquired immunodeficiency syndrome (PNAS 98: 5140-5 (2001)). Polymorphism in the HLA-B gene correlates with decreased occurrence of disease progression associated with HIV infections (PNAS 97: 2709-14 (2000)). Decreased expression of HLA-B protein may cause decreased NK cells function associated with leukemia (Blood 103: 3122-30 (2004)). Polymorphism in the HLA-B gene correlates with disease susceptibility associated with acquired immunodeficiency syndrome (Proc Natl Acad Sci USA 98: 5140-5 (2001)). Polymorphism in the HLA-B gene correlates with decreased occurrence of disease progression associated with HIV infections (Proc Natl Acad Sci USA 97: 2709-14 (2000)). (PhosphoSite®, Cell Signaling Technology (Danvers, Mass.), Human PSD™, Biobase Corporation, (Beverly, Mass.)).

HSP70 (P08107), phosphorylated at Y15, is among the proteins listed in this patent. HSP70, Heat shock 70 kDa protein 1A, an HSP70 family chaperone that modulates stress responses; gene polymorphism is associated with ankylosing spondylitis, celiac disease, and rheumatoid arthritis; altered expression is associated with lung cancer and diabetes. This protein has potential diagnostic and/or therapeutic implications based on the following findings. Decreased expression of HSPA1A in skeletal muscle correlates with abnormal glucose metabolic process associated with type II diabetes mellitus (Diabetes 51: 1102-9 (2002)). Decreased expression of HSPA1A protein may cause decreased apoptosis associated with colonic neoplasms (Cell Growth Differ 12: 419-26 (2001)). Increased expression of HSPA1A mRNA correlates with lung neoplasms (Int J Cancer 57: 486-90 (1994)). Decreased expression of HSPA1A protein may cause decreased apoptosis associated with adenocarcinoma (Cell Growth Differ 12: 419-26 (2001)). Abnormal expression of HSPA1A mRNA may correlate with abnormal response to drug associated with ovarian neoplasms (Biochem Pharmacol 58: 69-76 (1999)). (PhosphoSite®, Cell Signaling Technology (Danvers, Mass.), Human PSD™, Biobase Corporation, (Beverly, Mass.)).

HSP90B (P08238), phosphorylated at Y191, is among the proteins listed in this patent. HSP90B, Heat shock 90 kD protein 1 beta, involved in regulation of both cytochrome c-dependent apoptosis and antiapoptosis via Akt/PKB (AKT1), elevated expression is reported in patients with active systemic lupus erythematosus and glucocorticoid resistance. This protein has potential diagnostic and/or therapeutic implications based on the following findings. Decreased expression of HSP90AB1 mRNA may correlate with breast neoplasms (DNA Cell Biol 16: 1231-6 (1997)). Increased expression of HSP90AB1 protein may correlate with systemic lupus erythematosus (Immunology 97: 226-31 (1999)). (PhosphoSite®, Cell Signaling Technology (Danvers, Mass.), Human PSD™, Biobase Corporation, (Beverly, Mass.)).

IL2RG (P31785), phosphorylated at Y303, Y325, Y357, is among the proteins listed in this patent. IL2RG, Gamma subunit of the interleukin-2 receptor, a receptor component for several interleukins, activates JAK-STAT pathways to promote NK cell activity and T-cell proliferation; gene mutations cause X-linked severe combined immunodeficiency. This protein has potential diagnostic and/or therapeutic implications based on the following findings. Mutation in the IL2RG gene causes severe combined immunodeficiency (Cell 73: 147-57 (1993)). Lack of expression of IL2RG mRNA causes decreased cytokine and chemokine mediated signaling pathway associated with severe combined immunodeficiency (Eur J Immunol 24: 475-9 (1994)). Increased expression of IL2RG protein prevents decreased differentiation of lymphocytes associated with severe combined immunodeficiency (Science 288: 669-72 (2000)). Point mutation in the IL2RG gene causes decreased JAK-STAT cascade associated with severe combined immunodeficiency (Science 266: 1042-5 (1994)). Deletion mutation in the IL2RG gene causes decreased cytokine and chemokine mediated signaling pathway associated with severe combined immunodeficiency (J Immunol 153: 1310-7 (1994)). Splice site mutation in the IL2RG gene causes severe combined immunodeficiency (Hum Mol Genet 2: 1099-104 (1993)). Increased expression of IL2RG in brain correlates with chronic form of encephalitis (J Neuroimmunol 128: 9-15 (2002)). Frameshift mutation in the IL2RG gene causes decreased cytokine and chemokine mediated signaling pathway associated with severe combined immunodeficiency (Eur J Immunol 24: 475-9 (1994)). Nonsense mutation in the IL2RG gene causes severe combined immunodeficiency (Hum Mol Genet 2: 1099-104 (1993)). Mutation in the IL2RG gene correlates with increased occurrence of papillomavirus infections associated with severe combined immunodeficiency (Lancet 363: 2051-4 (2004)). Insertion mutation in the IL2RG gene causes severe combined immunodeficiency (J Clin Invest 95: 895-9 (1995)). Mutation in the IL2RG gene causes decreased cytokine and chemokine mediated signaling pathway associated with severe combined immunodeficiency (Blood 85: 38-42 (1995)). Increased expression of IL2RG in brain correlates with multiple sclerosis (J Immunol 165: 6576-82 (2000)). Mislocalization of IL2RG protein causes severe combined immunodeficiency (Hum Genet 107: 406-8 (2000)). (PhosphoSite®, Cell Signaling Technology (Danvers, Mass.), Human PSD™, Biobase Corporation, (Beverly, Mass.)).

IL6R (P08887), phosphorylated at Y464, is among the proteins listed in this patent. IL6R, Interleukin-6 (IL6) receptor alpha, binds IL6 and interacts with the signal transducer gp130 (human IL6ST), acts in leukocyte recruitment and activation, may contribute to AIDS progression and the pathogenesis of multiple myeloma. This protein has potential diagnostic and/or therapeutic implications based on the following findings. Increased expression of IL6R protein may correlate with lymphoma tumors associated with HIV infections (Leukemia 13: 634-40 (1999)). Antibody to IL6R may prevent increased lymphoma associated with HIV infections (Leukemia 13: 634-40 (1999)). Decreased expression of IL6R protein may correlate with carcinoma tumors associated with cervix neoplasms (J Immunol 165: 1939-48 (2000)). Increased expression of IL6R protein correlates with adenoma tumors associated with pituitary neoplasms (J Histochem Cytochem 42: 67-76 (1994)). Decreased expression of IL6R protein correlates with decreased cell proliferation associated with multiple myeloma (Blood 84: 3040-6 (1994)). Increased expression of IL6R in plasma cells correlates with multiple myeloma (Blood 96: 3880-6 (2000)). Increased expression of IL6R in brain may correlate with chronic form of encephalitis (J Neuroimmunol 128: 9-15 (2002)). Increased expression of IL6R mRNA correlates with increased occurrence of less severe form of breast neoplasms (Cancer 88: 2061-71 (2000)). Increased expression of IL6R in brain correlates with multiple sclerosis (J Immunol 165: 6576-82 (2000)). Increased expression of IL6R in plasma cells correlates with plasmacytoma (Blood 96: 3880-6 (2000)). Inhibition of IL6R antibody binding may prevent increased cell proliferation associated with lymphoma (Leukemia 13: 634-40 (1999)). Increased expression of IL6R in serum correlates with more severe form of multiple sclerosis (J Neuroimmunol 99: 218-23 (1999)). (PhosphoSite®, Cell Signaling Technology (Danvers, Mass.), Human PSD™, Biobase Corporation, (Beverly, Mass.)).

IRS-2 (Q9Y4H2), phosphorylated at Y978, is among the proteins listed in this patent. IRS-2, Insulin receptor substrate 2, binds various kinases and mediates signal transduction through receptors for insulin, integrin, and cytokines, may be associated with type 2 diabetes and carcinoma cell invasion; mouse Irs2 is associated with type 2 diabetes. This protein has potential diagnostic and/or therapeutic implications based on the following findings. Polymorphism in the IRS2 gene correlates with insulin resistance associated with polycystic ovary syndrome (J Clin Endocrinol Metab 87: 4297-300 (2002)). Polymorphism in the IRS2 gene correlates with increased severity of insulin resistance associated with polycystic ovary syndrome (Diabetes 50: 2164-8 (2001)). Increased expression of IRS2 protein may cause increased cell proliferation associated with pancreatic neoplasms (Cancer Res 58: 4250-4 (1998)). Polymorphism in the IRS2 gene correlates with more severe form of insulin resistance (Diabetes: S304-7 (2002)). Missense mutation in the IRS2 gene may correlate with decreased beta cells function associated with type II diabetes mellitus (Hum Mol Genet 9: 2517-21 (2000)). Polymorphism in the IRS2 gene correlates with glucose intolerance (Hum Genet 113: 34-43 (2003)). Increased phosphorylation of IRS2 may cause increased integrin-mediated signaling pathway associated with carcinoma (Mol. Cell Biol 21: 5082-93 (2001)). Increased phosphorylation of IRS2 may cause invasive form of carcinoma (Mol. Cell Biol 21: 5082-93 (2001)). Increased phosphorylation of IRS2 may cause invasive form of carcinoma (MCB 21: 5082-93 (2001)). Increased expression of IRS2 mRNA may correlate with increased cell migration associated with breast neoplasms (Oncogene 20: 7318-25 (2001)). Decreased phosphorylation of IRS2 may cause insulin resistance (Diabetes 51: 1052-9 (2002)). Increased expression of IRS2 mRNA may correlate with malignant form of breast neoplasms (Oncogene 20: 7318-25 (2001)). Missense mutation in the IRS2 gene correlates with increased occurrence of type II diabetes mellitus associated with obesity (Hum Mol Genet 9: 2517-21 (2000)). Increased phosphorylation of IRS2 may correlate with malignant form of breast neoplasms (Oncogene 20: 7318-25 (2001)). Increased phosphorylation of IRS2 may correlate with increased cell migration associated with breast neoplasms (Oncogene 20: 7318-25 (2001)). Increased phosphorylation of IRS2 may cause invasive form of carcinoma (Mol Cell Biol 21: 5082-93 (2001)). Polymorphism in the IRS2 gene correlates with more severe form of insulin resistance (Diabetes 50: 2164-8 (2001)). Increased phosphorylation of IRS2 may cause increased integrin-mediated signaling pathway associated with carcinoma (Mol Cell Biol. 21: 5082-93 (2001)). Polymorphism in the IRS2 gene correlates with increased severity of insulin resistance associated with obesity (Diabetes: S304-7 (2002)). Increased phosphorylation of IRS2 may cause invasive form of carcinoma (Mol Cell Biol. 21: 5082-93 (2001)). Missense mutation in the IRS2 gene may correlate with increased severity of insulin resistance associated with type II diabetes mellitus (Hum Mol Genet 9: 2517-21 (2000)). Decreased phosphorylation of IRS2 may cause insulin resistance associated with glucose intolerance (Diabetes 51: 1052-9 (2002)). Increased phosphorylation of IRS2 may cause increased integrin-mediated signaling pathway associated with carcinoma (MCB 21: 5082-93 (2001)). Increased phosphorylation of IRS2 may cause increased integrin-mediated signaling pathway associated with carcinoma (Mol. Cell. Biol. 21: 5082-93 (2001)). Increased phosphorylation of IRS2 may cause invasive form of carcinoma (Mol. Cell. Biol. 21: 5082-93 (2001)). Increased phosphorylation of IRS2 may cause increased integrin-mediated signaling pathway associated with carcinoma (Mol Cell Biol 21: 5082-93 (2001)). Increased expression of IRS2 mRNA correlates with pancreatic neoplasms (Cancer Res 58: 4250-4 (1998)). (PhosphoSite®, Cell Signaling Technology (Danvers, Mass.), Human PSD™, Biobase Corporation, (Beverly, Mass.)).

Jak2 (O60674), phosphorylated at Y382, Y423, Y435, is among the proteins listed in this patent. Jak2, Janus kinase 2, protein tyrosine kinase that functions in cytokine-induced JAK-STAT signaling, activated in response to growth hormone (GH) and IFN-gamma (IFNG), inhibits apoptosis; gene translocation is associated with chronic myelogenous leukemia. This protein has potential diagnostic and/or therapeutic implications based on the following findings. Increased phosphorylation of JAK2 correlates with increased anti-apoptosis associated with acute promyelocytic leukemia (Leukemia 15: 1176-84 (2001)). Increased phosphorylation of JAK2 correlates with increased differentiation of granulocytes associated with acute promyelocytic leukemia (Leukemia 15: 1176-84 (2001)). Decreased phosphorylation of JAK2 may prevent increased cell proliferation associated with breast neoplasms (JBC 275: 33937-44 (2000)). Decreased phosphorylation of JAK2 may prevent increased cell proliferation associated with breast neoplasms (J Biol Chem 275: 33937-44 (2000)). Increased phosphorylation of JAK2 may correlate with increased cell proliferation associated with breast neoplasms (J Biol Chem 273: 31308-16 (1998)). Amplification of the JAK2 gene correlates with mediastinal neoplasms associated with B-cell lymphoma (Blood 104: 543-9 (2004)). Increased phosphorylation of JAK2 may cause increased cell proliferation associated with acute erythroblastic leukemia (Blood 93: 2369-79 (1999)). Increased expression of JAK2 mRNA correlates with B-cell lymphoma associated with mediastinal neoplasms (Blood 104: 543-9 (2004)). Increased phosphorylation of JAK2 may cause increased cell proliferation associated with chronic myeloid leukemia (Blood 93: 2369-79 (1999)). Increased phosphorylation of JAK2 may correlate with mediastinal neoplasms associated with B-cell lymphoma (Blood 104: 543-9 (2004)). Increased phosphorylation of JAK2 may cause increased anti-apoptosis associated with prostatic neoplasms (FEBS Lett 488: 179-184 (2001)). Induced inhibition of the signal transducer activity of JAK2 may cause increased apoptosis associated with hepatocellular carcinoma (Nat Genet 28: 29-35 (2001)). JAK2 mutant protein correlates with late onset form of chronic myeloid leukemia (Blood 90: 2535-40 (1997)). Amplification of the JAK2 gene correlates with increased severity of non-Hodgkin's lymphoma associated with B-cell lymphoma (Oncogene 22: 1425-9 (2003)). Translocation of the JAK2 gene correlates with early onset form of acute T-cell leukemia (Science 278: 1309-12 (1997)). Increased signal transducer activity of JAK2 correlates with hepatocellular carcinoma (Nat Genet 28: 29-35 (2001)). Increased phosphorylation of JAK2 may cause increased cell proliferation associated with acute megakaryocytic leukemia (Blood 93: 2369-79 (1999)). Increased phosphorylation of JAK2 may correlate with increased cell proliferation associated with myeloid leukemia (Leukemia 11: 1941-9 (1997)). Translocation of the JAK2 gene correlates with late onset form of chronic myeloid leukemia (Blood 90: 2535-40 (1997)). Increased phosphorylation of JAK2 may correlate with increased response to hormone stimulus associated with prostatic neoplasms (Mol Cell Endocrinol 220: 109-23 (2004)). Induced inhibition of JAK2 protein may prevent abnormal regulation of cell shape associated with breast neoplasms (Endocrinology 141: 1571-84 (2000)). Increased expression of JAK2 mRNA correlates with mediastinal neoplasms associated with B-cell lymphoma (Blood 104: 543-9 (2004)). Induced inhibition of JAK2 protein may prevent increased anti-apoptosis associated with plasmacytoma (Eur J Immunol 29: 3945-50 (1999)). Increased phosphorylation of JAK2 may cause increased cell proliferation associated with prostatic neoplasms (FEBS Lett 488: 179-184 (2001)). Amplification of the JAK2 gene correlates with B-cell lymphoma associated with mediastinal neoplasms (Blood 104: 543-9 (2004)). Translocation of the JAK2 gene correlates with early onset form of acute lymphocytic leukemia (L1) (Blood 90: 253540 (1997)). Increased phosphorylation of JAK2 may correlate with B-cell lymphoma associated with mediastinal neoplasms (Blood 104: 543-9 (2004)). JAK2 mutant protein may cause increased cell proliferation associated with acute T-cell leukemia (Science 278: 1309-12 (1997)). Increased phosphorylation of JAK2 may correlate with increased cell proliferation associated with breast neoplasms (JBC 273: 31308-16 (1998)). Induced inhibition of JAK2 protein prevents increased cell proliferation associated with acute lymphocytic leukemia (Nature 379: 645-8 (1996)). (PhosphoSite®, Cell Signaling Technology (Danvers, Mass.), Human PSD™, Biobase Corporation, (Beverly, Mass.)).

KI-67 (P46013), phosphorylated at Y340, is among the proteins listed in this patent. KI-67, Ki-67 antigen, induces chromatin compaction, acts in cell proliferation, expression is altered in neoplasms including osteosarcoma and prostate, breast and esophageal cancer; gene is mutated in cervical, colon and lung carcinoma cell lines. This protein has potential diagnostic and/or therapeutic implications based on the following findings. Increased expression of MKI67 protein correlates with increased occurrence of recurrence associated with lung neoplasms (Eur J Cancer: 363-5 (1993)). Increased expression of MKI67 protein correlates with disease progression associated with multiple myeloma (Anticancer Res 20: 4619-25 (2000)). Increased expression of MKI67 protein may correlate with increased occurrence of recurrence associated with breast neoplasms (Cancer 71: 3926-31 (1993)). Increased expression of MKI67 protein correlates with increased cell proliferation associated with breast ductal carcinoma (Anticancer Res 22: 295-8 (2002)). Increased expression of MKI67 protein correlates with increased cell proliferation associated with breast ductal carcinoma (Cancer 82: 2373-81 (1998)). Increased expression of MKI67 protein correlates with increased occurrence of death associated with breast neoplasms (Cancer 97: 1321-31 (2003)). Increased expression of MKI67 protein correlates with decreased cell differentiation associated with breast neoplasms (Anticancer Res 11: 2015-21 (1991)). Increased expression of MKI67 in lymphocytes correlates with increased proliferation of T-lymphocytes associated with HIV infections (Blood 95: 249-55 (2000)). Increased expression of MKI67 protein correlates with increased occurrence of death associated with breast neoplasms (J Natl Cancer Inst 91: 271-8 (1999)). Increased expression of MKI67 protein correlates with increased cell proliferation associated with breast neoplasms (J Natl Cancer Inst 91: 271-8 (1999)). Decreased expression of MKI67 protein correlates with decreased occurrence of death associated with cervix neoplasms (Eur J Cancer 37: 1104-10 (2001)). Increased expression of MKI67 protein may correlate with increased occurrence of death associated with breast neoplasms (Cancer 71: 3926-31 (1993)). Increased expression of MKI67 protein correlates with increased proliferation of keratinocytes associated with psoriasis (J Exp Med 182: 2057-68 (1995)). Increased expression of MKI67 protein may correlate with trisomy associated with rheumatoid arthritis (Hum Genet 96: 6514 (1995)).

Increased expression of MKI67 protein correlates with osteosarcoma associated with bone neoplasms (Cancer 75: 806-14 (1995)). Increased expression of MKI67 protein correlates with lymphatic metastasis associated with breast neoplasms (Anticancer Res 11: 2015-21 (1991)). Increased expression of MKI67 protein correlates with increased occurrence of death associated with lung neoplasms (Cancer 89: 1457-65 (2000)). Increased expression of MKI67 protein correlates with carcinoma in situ associated with breast neoplasms (Cancer Res 52: 2597-602 (1992)). Increased expression of MKI67 protein correlates with increased cell proliferation associated with breast ductal carcinoma (Br J Cancer 78: 788-94 (1998)). Increased expression of MKI67 protein correlates with aneuploidy associated with breast neoplasms (Anticancer Res 11: 2015-21 (1991)). Increased expression of MKI67 protein correlates with increased cell proliferation associated with colonic neoplasms (Cancer Lett 115: 229-34 (1997)). Increased expression of MKI67 protein correlates with increased occurrence of recurrence associated with non-small-cell lung carcinoma (Eur J Cancer: 363-5 (1993)). Increased expression of MKI67 protein correlates with increased angiogenesis associated with breast ductal carcinoma (Anticancer Res 19: 3269-74 (1999)). Increased expression of MKI67 protein correlates with increased cell proliferation associated with breast ductal carcinoma (Anticancer Res 19: 3269-74 (1999)). Increased expression of MKI67 protein correlates with chondroma associated with bone neoplasms (Cancer 75: 806-14 (1995)). Increased expression of MKI67 protein may correlate with increased cell proliferation associated with rheumatoid arthritis (Hum Genet 96: 651-4 (1995)). Increased expression of MKI67 protein correlates with increased immune response associated with HIV infections (Blood 95: 249-55 (2000)). Increased expression of MKI67 protein correlates with hyperplasia associated with psoriasis (J Exp Med 182: 2057-68 (1995)). Increased expression of MKI67 protein correlates with increased occurrence of recurrence associated with breast neoplasms (Anticancer Res 19: 4033-7 (1999)). Increased expression of MKI67 protein correlates with more severe form of bone neoplasms (Cancer 75: 806-14 (1995)). Increased expression of MKI67 protein correlates with breast ductal carcinoma associated with breast neoplasms (Cancer Res 52: 2597-602 (1992)). Increased expression of MKI67 protein correlates with increased apoptosis associated with breast ductal carcinoma (Br J Cancer 78: 788-94 (1998)). Increased expression of MKI67 protein correlates with increased occurrence of recurrence associated with breast neoplasms (Cancer 97: 1321-31 (2003)). (PhosphoSite®, Cell Signaling Technology (Danvers, Mass.), Human PSD™, Biobase Corporation, (Beverly, Mass.)).

Kit (P10721), phosphorylated at Y609, is among the proteins listed in this patent. Kit, V-kit Hardy-Zuckerman 4 feline sarcoma viral oncogene homolog, tyrosine kinase that binds stem cell factor, involved in melanocyte development, inhibits apoptosis, possible therapeutic target in neoplasms; gene mutations cause piebaldism and mastocytosis. This protein has potential diagnostic and/or therapeutic implications based on the following findings. Decreased expression of KIT mRNA correlates with decreased positive regulation of transcription from RNA polymerase II promoter associated with melanoma (EMBO J. 17: 4358-69 (1998)). Induced stimulation of the stem cell factor receptor activity of KIT causes increased cell-matrix adhesion associated with myeloid leukemia (Leukemia 12: 1375-82 (1998)). Increased protein-tyrosine kinase activity of KIT correlates with neoplastic cell transformation associated with gastrointestinal neoplasms (Science 279: 577-80 (1998)). Point mutation in the KIT gene may cause abnormal transmembrane receptor protein serine/threonine kinase signaling pathway associated with acute myelocytic leukemia (Blood 97: 3559-67 (2001)). Point mutation in the KIT gene may cause decreased apoptosis associated with acute myelocytic leukemia (Blood 97: 3559-67 (2001)). Deletion mutation in the KIT gene correlates with increased occurrence of neoplasm metastasis associated with gastrointestinal neoplasms (Int J Cancer 106: 887-95 (2003)). Gain of function mutation in the KIT gene correlates with autosomal dominant form of gastrointestinal neoplasms (Cancer 92: 657-62 (2001)). Induced inhibition of the protein-tyrosine kinase activity of KIT prevents disease progression associated with acute myelocytic leukemia (Blood 101: 2960-2 (2003)). Gain of function mutation in the KIT gene correlates with increased severity of leukocytosis associated with acute myelocytic leukemia (Blood 102: 1474-9 (2003)). Increased expression of KIT mRNA correlates with early stage or low grade form of ovarian neoplasms (Int J Cancer 89: 242-50 (2000)). Increased expression of KIT protein may correlate with T-cell lymphoma (Leukemia 15: 1641-9 (2001)). Increased expression of KIT protein correlates with carcinoma associated with cervix neoplasms (Cancer Res 61: 6281-9 (2001)). Increased expression of KIT protein correlates with acute myelocytic leukemia (Leukemia 8: 258-63 (1994)). Gain of function mutation in the KIT gene correlates with decreased response to drug associated with acute myelocytic leukemia (Blood 102: 1474-9 (2003)). Induced inhibition of the protein-tyrosine kinase activity of KIT may prevent decreased induction of apoptosis associated with gastrointestinal neoplasms (Oncogene 20: 5054-8 (2001)). Point mutation in the KIT gene may cause increased cell proliferation associated with acute myelocytic leukemia (Blood 97: 3559-67 (2001)). Induced inhibition of the protein-tyrosine kinase activity of KIT may prevent decreased apoptosis associated with myeloid leukemia (Blood 97: 1413-21 (2001)). Increased expression of KIT mRNA may prevent neoplasm invasiveness associated with melanoma (EMBO J. 17: 4358-69 (1998)). Increased expression of KIT in astrocytes may cause increased apoptosis associated with HIV infections (Proc Natl Acad Sci USA 94: 3954-9 (1997)). MRNA instability of KIT correlates with decreased positive regulation of cell proliferation associated with acute myelocytic leukemia (Cancer Res 53: 3638-42 (1993)). Increased stem cell factor receptor activity of KIT may cause increased cell proliferation associated with acute megakaryocytic leukemia (Blood 85: 1220-8 (1995)). Increased expression of KIT mRNA may prevent neoplasm metastasis associated with melanoma (EMBO J. 17: 4358-69 (1998)). Increased expression of KIT mRNA may prevent neoplasm invasiveness associated with melanoma (EMBO 17: 4358-69 (1998)). Increased expression of KIT protein correlates with drug-resistant form of myeloid leukemia (Leukemia 11: 1850-7 (1997)). Induced inhibition of the transmembrane receptor protein tyrosine kinase activity of KIT may prevent disease progression associated with myeloid leukemia (Blood 98: 241-3 (2001)). Induced inhibition of the protein-tyrosine kinase activity of KIT may prevent increased cell proliferation associated with colorectal neoplasms (Cancer Res 62: 4879-83 (2002)). Induced inhibition of the protein-tyrosine kinase activity of KIT causes increased occurrence of necrosis associated with gastrointestinal neoplasms (Br J Cancer 89: 460-4 (2003)). Decreased expression of KIT protein correlates with neoplasm invasiveness associated with skin neoplasms (Int J Cancer 52: 197-201 (1992)). Increased expression of KIT protein correlates with carcinoma associated with ovarian neoplasms (Cancer 98: 758-64 (2003)). Induced stimulation of the stem cell factor receptor activity of KIT causes increased cell proliferation associated with myeloid leukemia (Leukemia 12: 1375-82 (1998)). Decreased expression of KIT protein correlates with neoplastic cell transformation associated with melanoma (Int J Cancer 52: 197-201 (1992)). Induced inhibition of the protein-tyrosine kinase activity of KIT may prevent increased cell proliferation associated with small cell carcinoma (Cancer Res 62: 6304-11 (2002)). Induced inhibition of the transmembrane receptor protein tyrosine kinase activity of KIT may correlate with gynecomastia (Lancet 361: 1954-6 (2003)). Increased expression of KIT protein correlates with increased cell proliferation associated with acute myelocytic leukemia (J Cell Physiol 154: 410-8 (1993)). Increased expression of KIT protein correlates with increased drug export associated with myeloid leukemia (Leukemia 11: 1850-7 (1997)). Increased expression of KIT mRNA correlates with carcinoma associated with colorectal neoplasms (J Cell Physiol 172: 1-11 (1997)). Decreased expression of KIT mRNA correlates with decreased positive regulation of transcription from RNA polymerase II promoter associated with melanoma (EMBO 17: 4358-69 (1998)). Lack of expression of KIT mRNA may cause neoplasm metastasis associated with skin neoplasms (Oncogene 13: 2339-47 (1996)). Induced inhibition of the protein-tyrosine kinase activity of KIT may prevent increased anti-apoptosis associated with colorectal neoplasms (Cancer Res 62: 4879-83 (2002)). Decreased expression of KIT mRNA correlates with decreased positive regulation of transcription from RNA polymerase II promoter associated with melanoma (EMBO J 17: 4358-69 (1998)). Decreased expression of KIT protein correlates with neoplasm invasiveness associated with melanoma (Int J Cancer 52: 197-201 (1992)). Induced inhibition of the protein-tyrosine kinase activity of KIT may prevent increased activation of MAPK activity associated with myeloid leukemia (Blood 97: 1413-21 (2001)). Induced inhibition of the protein-tyrosine kinase activity of KIT may prevent increased protein amino acid autophosphorylation associated with myeloid leukemia (Blood 97: 1413-21 (2001)). Lack of expression of KIT mRNA may cause neoplasm metastasis associated with melanoma (Oncogene 13: 2339-47 (1996)). Induced inhibition of the stem cell factor receptor activity of KIT may cause increased anti-apoptosis associated with colonic neoplasms (Cancer Res 61: 2200-6 (2001)). Lack of expression of KIT mRNA may cause increased occurrence of malignant form of melanoma (Oncogene 13: 2339-47 (1996)). Decreased tyrosine phosphorylation of KIT may prevent abnormal signal transduction associated with gastrointestinal neoplasms (Oncogene 20: 5054-8 (2001)). Induced inhibition of the transmembrane receptor protein tyrosine kinase activity of KIT may cause abnormal transmembrane receptor protein tyrosine kinase signaling pathway associated with hypopigmentation (Cancer 98: 2483-7 (2003)). Induced inhibition of the protein-tyrosine kinase activity of KIT may prevent increased cell proliferation associated with lung neoplasms (Cancer Res 62: 6304-11 (2002)). Alternative form of KIT mRNA may cause acute myelocytic leukemia (Cancer Lett 116: 253-8 (1997)). Induced inhibition of the protein-tyrosine kinase activity of KIT may prevent increased peptidyl-tyrosine phosphorylation associated with gastrointestinal neoplasms (Oncogene 20: 5054-8 (2001)). Induced inhibition of the protein-tyrosine kinase activity of KIT prevents decreased apoptosis associated with bone neoplasms (J Natl Cancer Inst 94: 1673-9 (2002)). Missense mutation in the KIT gene correlates with increased severity of neoplasm invasiveness associated with gastrointestinal neoplasms (Cancer Res 59: 4297-300 (1999)). Induced inhibition of the protein-tyrosine kinase activity of KIT prevents disease progression associated with gastrointestinal neoplasms (Lancet 358: 1421-3 (2001)). Increased expression of KIT mRNA may prevent neoplasm metastasis associated with melanoma (EMBO 17: 4358-69 (1998)). Increased expression of KIT protein correlates with glandular and epithelial neoplasms associated with ovarian neoplasms (Int J Cancer 89: 242-50 (2000)). Point mutation in the KIT gene may cause abnormal regulation of transcription associated with acute myelocytic leukemia (Blood 97: 3559-67 (2001)). Lack of expression of KIT protein correlates with breast neoplasms (Br J Cancer 73: 1233-6 (1996)). Decreased expression of KIT protein correlates with neoplastic cell transformation associated with skin neoplasms (Int J Cancer 52: 197-201 (1992)). Induced inhibition of the protein-tyrosine kinase activity of KIT may prevent increased cell proliferation associated with myeloid leukemia (Blood 97: 1413-21 (2001)). Induced inhibition of the protein-tyrosine kinase activity of KIT prevents increased severity of Ewing's sarcoma associated with bone neoplasms (J Natl Cancer Inst 94: 1673-9 (2002)). Induced inhibition of the protein-tyrosine kinase activity of KIT prevents increased cell proliferation associated with gastrointestinal neoplasms (Br J Cancer 89: 460-4 (2003)). Mutation in the KIT gene correlates with urticaria pigmentosa (Nat Genet 12: 312-4 (1996)). Increased expression of KIT mRNA may prevent increased cell proliferation associated with breast neoplasms (Anticancer Res 16: 3397-402 (1996)). Increased expression of KIT in astrocytes may cause increased apoptosis associated with HIV infections (PNAS 94: 3954-9 (1997)). Increased expression of KIT protein may cause increased cell proliferation associated with myeloid leukemia (Leukemia 7: 426-34 (1993)). Induced inhibition of the transmembrane receptor protein tyrosine kinase activity of KIT may prevent disease progression associated with myeloid leukemia (Blood 102: 795-801 (2003)). Point mutation in the KIT gene may cause abnormal regulation of tyrosine phosphorylation of Stat3 protein associated with acute myelocytic leukemia (Blood 97: 3559-67 (2001)). Missense mutation in the KIT gene correlates with increased occurrence of gastrointestinal hemorrhage associated with gastrointestinal neoplasms (Cancer Res 59: 4297-300 (1999)). Decreased protein-tyrosine kinase activity of KIT correlates with neoplastic cell transformation associated with melanoma (Mol Bio Cell 3: 197-209 (1992)). Lack of expression of KIT protein may cause decreased apoptosis associated with melanoma (J Cell Physiol 173: 275-8 (1997)). Induced stimulation of the stem cell factor receptor activity of KIT may cause increased inflammatory response associated with inflammatory bowel diseases (Gut 38: 104-14 (1996)). Lack of expression of KIT mRNA correlates with breast neoplasms (Int J Cancer 52: 713-7 (1992)). Increased expression of KIT mRNA may prevent neoplasm metastasis associated with melanoma (EMBO J 17: 4358-69 (1998)). Increased expression of KIT in myeloid cells correlates with hypersensitivity (J Immunol 161: 5079-86 (1998)). Increased expression of KIT protein correlates with acute form of myeloid leukemia (Blood 92: 596-9 (1 998)). Increased expression of KIT mRNA may not prevent increased cell proliferation associated with ovarian neoplasms (Exp Cell Res 273: 95-106 (2002)). Induced inhibition of the protein-tyrosine kinase activity of KIT prevents increased protein amino acid phosphorylation associated with bone neoplasms (J Natl Cancer Inst 94: 1673-9 (2002)). Induced stimulation of the stem cell factor receptor activity of KIT may cause increased cell migration associated with small cell carcinoma (Cancer Res 53: 1709-14 (1993)). Induced stimulation of the stem cell factor receptor activity of KIT may cause increased mast cell activation associated with inflammatory bowel diseases (Gut 38: 104-14 (1996)). Increased stem cell factor receptor activity of KIT may prevent abnormal cell proliferation associated with melanoma (Oncogene 8: 2221-9 (1993)). Decreased expression of KIT protein correlates with melanoma associated with skin neoplasms (Int J Cancer 52: 197-201 (1992)). Gain of function mutation in the KIT gene may cause increased tyrosine phosphorylation of Stat3 protein associated with gastrointestinal neoplasms (Anticancer Res 23: 2253-60 (2003)). Lack of expression of KIT mRNA may correlate with melanoma (Anticancer Res 14: 1759-65 (1994)). Increased expression of KIT in astrocytes may cause increased apoptosis associated with HIV infections (Proc Natl Acad Sci USA 94: 3954-9 (1997)). Missense mutation in the KIT gene correlates with increased incidence of recurrence associated with gastrointestinal neoplasms (Cancer Res 59: 4297-300 (1999)). Decreased stem cell factor receptor activity of KIT may prevent abnormal cell-cell signaling associated with cervix neoplasms (Cancer Res 61: 6281-9 (2001)). Missense mutation in the KIT gene correlates with increased occurrence of necrosis associated with gastrointestinal neoplasms (Cancer Res 59: 4297-300 (1999)). Induced inhibition of the protein-tyrosine kinase activity of KIT does not prevent disease progression associated with acute myelocytic leukemia (Cancer 97: 2760-6 (2003)). Increased stem cell factor receptor activity of KIT may cause abnormal signal transduction associated with melanoma (Oncogene 8: 2221-9 (1993)). Increased stem cell factor receptor activity of KIT may prevent decreased apoptosis associated with skin neoplasms (Oncogene 13: 2339-47 (1996)). Lack of expression of KIT mRNA may correlate with melanoma associated with skin neoplasms (Oncogene 13: 2339-47 (1996)). Alternative form of KIT mRNA correlates with gastrointestinal neoplasms (Cancer Lett 115: 257-61 (1997)). Abnormal expression of KIT protein correlates with melanoma associated with skin neoplasms (J Cell Biochem 83: 364-72 (2001)). Alternative form of KIT mRNA may correlate with colonic neoplasms (Cancer Res 54: 272-5 (1994)). Increased expression of KIT mRNA may correlate with Ewing's sarcoma associated with bone neoplasms (Blood 91: 2397-405 (1998)). Increased protein-tyrosine kinase activity of KIT correlates with urticaria pigmentosa (Nat Genet 12: 3124 (1996)). Lack of expression of KIT protein correlates with increased occurrence of death associated with ovarian neoplasms (Int J Cancer 89: 242-50 (2000)). (PhosphoSite®, Cell Signaling Technology (Danvers, Mass.), Human PSD™, Biobase Corporation, (Beverly, Mass.)).

Lasp-1 (Q14847), phosphorylated at Y52, Y57, Y 183, is among the proteins listed in this patent. Lasp-1, LIM and SH3 protein 1, a LIM and SH3 domain-containing protein, binds actin, may regulate cytoskeletal organization at membrane extensions; gene may be amplified and overexpressed in breast carcinoma, gene is fused to MLL in acute myeloid leukemia. This protein has potential diagnostic and/or therapeutic implications based on the following findings. Increased expression of LASP1 mRNA correlates with carcinoma tumors associated with breast neoplasms (FEBS Lett 373: 245-9 (1995)). Translocation of the LASP1 gene correlates with acute form of myeloid leukemia (Oncogene 22: 157-60 (2003)). Amplification of the LASP1 gene may correlate with carcinoma tumors associated with breast neoplasms (Genomics 28: 367-76 (1 995)). Amplification of the LASP1 gene correlates with carcinoma tumors associated with breast neoplasms (Cancer Res 56: 3886-90 (1996)). Increased expression of LASP1 mRNA may correlate with carcinoma tumors associated with breast neoplasms (Genomics 28: 367-76 (1995)). (PhosphoSite®, Cell Signaling Technology (Danvers, Mass.), Human PSD™, Biobase Corporation, (Beverly, Mass.)).

Lck (P06239), phosphorylated at Y262, Y263, Y413, is among the proteins listed in this patent. Lck, Lymphocyte-specific protein tyrosine kinase, involved in signaling through Ras and MAPK pathways, activated in response to T-cell receptor engagement, apoptotic activator of CASP8, may be therapeutic for HIV infection and metastatic cancers. This protein has potential diagnostic and/or therapeutic implications based on the following findings. LCK epitope may prevent increased occurrence of malignant form of colonic neoplasms (Eur J Immunol 31: 323-32 (2001)). Decreased expression of LCK protein may correlate with lung neoplasms (Blood 89: 212-8 (1997)). Induced inhibition of LCK protein may prevent increased cell proliferation associated with small cell carcinoma (Cancer Res 58: 4660-6 (1998)). Increased expression of LCK in B-lymphocytes may correlate with Epstein-Barr virus infections (Blood 91: 3390-6 (1998)). Increased oxidation of LCK correlates with HIV infections (J Clin Invest 98: 1290-7 (1996)). Alternative form of LCK protein correlates with acute T-cell leukemia (Cell Growth Differ 5: 659-66 (1994)). Increased expression of LCK protein correlates with increased occurrence of malignant form of brain neoplasms (Eur J Immunol 31: 323-32 (2001)). Decreased expression of LCK in lymphocytes correlates with renal cell carcinoma associated with kidney neoplasms (Cancer Res 53: 5613-6 (1993)). LCK epitope may prevent increased occurrence of malignant form of esophageal neoplasms (Eur J Immunol 31: 323-32 (2001)). Increased expression of LCK in thymus correlates with acute T-cell leukemia (J Exp Med 174: 867-73 (1991)). Mislocalization of LCK protein may correlate with HIV infections (J Immunol 158: 2017-24 (1997)). Induced inhibition of LCK protein may correlate with HIV infections (JBC 271: 6333-41 (1996)). Induced inhibition of LCK protein may correlate with HIV infections (J Biol Chem 271: 6333-41 (1996)). LCK epitope may prevent increased occurrence of malignant form of lung neoplasms (Eur J Immunol 31: 323-32 (2001)). Decreased expression of LCK in resting T-cells may cause decreased active T-cells function associated with type I diabetes mellitus (J Immunol 165: 5874-83 (2000)). LCK epitope may prevent increased occurrence of malignant form of neoplasms (Int J Cancer 94: 237-42 (2001)). LCK epitope may prevent increased occurrence of malignant form of neoplasms (Eur J Immunol 31: 323-32 (2001)). Increased expression of LCK in B-lymphocytes correlates with viral cell transformation associated with chronic B-cell leukemia (Blood 91: 3390-6 (1998)). Translocation of the LCK promoter correlates with acute T-cell leukemia (J Exp Med 174: 867-73 (1991)). Increased phosphorylation of LCK may correlate with HIV infections (Immunology 95: 214-8 (1998)). Alternative form of LCK protein correlates with acute B-cell leukemia (Cell Growth Differ 5: 659-66 (1994)). Alternative form of LCK protein correlates with myeloid leukemia (Cell Growth Differ 5: 659-66 (1994)). Alternative form of LCK mRNA correlates with leukemia (Cell Growth Differ 5: 659-66 (1994)). Decreased expression of LCK in T-lymphocytes may correlate with renal cell carcinoma associated with kidney neoplasms (J Immunol 159: 3057-67 (1997)). Increased expression of LCK in T-lymphocytes correlates with acute T-cell leukemia (J. Exp Med 174: 867-73 (1991)). Decreased expression of LCK in lymphocytes correlates with renal cell carcinoma (Cancer Res 53: 5613-6 (1993)). Decreased expression of LCK in T-lymphocytes may correlate with renal cell carcinoma (J Immunol 159: 3057-67 (1997)). Increased expression of LCK in B-lymphocytes correlates with chronic B-cell leukemia (Blood 91: 3390-6 (1998)). Abnormal tyrosine phosphorylation of LCK correlates with B-cell lymphoma (J Immunol 155: 1382-92 (1995)). Increased expression of LCK in T-lymphocytes may prevent HIV infections (Clin Exp Immunol 133: 78-90 (2003)). (PhosphoSite®, Cell Signaling Technology (Danvers, Mass.), Human PSD™, Biobase Corporation, (Beverly, Mass.)).

L-plastin (P13796), phosphorylated at Y1 18, Y299, Y374, is among the proteins listed in this patent. L-plastin, Lymphocyte cytosolic protein 1, an F-actin binding protein that acts in F-actin microspike and bundle formations and GPCR signaling pathway; corresponding gene is translocated in B-Cell non-Hodgkin lymphoma, expression is increased in various cancers. This protein has potential diagnostic and/or therapeutic implications based on the following findings. Increased expression of LCP1 mRNA correlates with fibrosarcoma (JBC 268: 2781-92 (1993)). Increased expression of LCP1 mRNA correlates with ovarian neoplasms (JBC 268: 2781-92 (1993)). Increased expression of LCP1 mRNA correlates with breast neoplasms (J Biol Chem 268: 2781-92 (1993)). Increased expression of LCP1 mRNA correlates with fibrosarcoma (J Biol Chem 268: 2781-92 (1993)). Increased expression of LCP1 protein may correlate with hormone-dependent neoplasms associated with breast neoplasms (DNA Cell Biol 19: 1-7 (2000)). Increased expression of LCP1 mRNA correlates with breast neoplasms (JBC 268: 2781-92 (1993)). Increased expression of LCP1 protein may correlate with hormone-dependent neoplasms associated with prostatic neoplasms (DNA Cell Biol 19: 1-7 (2000)). Increased expression of LCP1 mRNA correlates with choriocarcinoma (J Biol Chem 268: 2781-92 (1993)). Increased expression of LCP1 mRNA correlates with choriocarcinoma (JBC 268: 2781-92 (1993)). Increased expression of LCP1 in epithelium/epithelial cells correlates with breast neoplasms (Anticancer Res 20: 3177-82 (2000)). Increased expression of LCP1 mRNA correlates with ovarian neoplasms (J Biol Chem 268: 2781-92 (1993)) (PhosphoSite®, Cell Signaling Technology (Danvers, Mass.), Human PSD™, Biobase Corporation, (Beverly, Mass.)).

LRRK2 (Q5S007), phosphorylated at Y707, is among the proteins listed in this patent. LRRK2, Leucine-rich repeat kinase 2 (dardarin), a member of the ROCO protein family, contains a MAPKKK class protein kinase domain; mutations are associated with a familial form of autosomal dominant Parkinson disease. This protein has potential diagnostic and/or therapeutic implications based on the following findings. Missense mutation in the LRRK2 gene may cause dementia associated with Parkinsonian disorders (Neuron 44: 601-7 (2004)). Missense mutation in the LRRK2 gene causes Parkinson disease (Neuron 44: 595-600 (2004)). LRRK2 map position correlates with autosomal dominant form of Parkinson disease (Am J Hum Genet 74: 11-9 (2004)). Missense mutation in the LRRK2 gene may cause nerve degeneration associated with Parkinsonian disorders (Neuron 44: 601-7 (2004)). (PhosphoSite®, Cell Signaling Technology (Danvers, Mass.), Human PSD™, Biobase Corporation, (Beverly, Mass.)).

LSD1 (O60341), phosphorylated at Y363, is among the proteins listed in this patent. LSD1, KIAA0601 protein, a riboflavin-binding protein, member of a FAD dependent enzyme superfamily, component of the HDAC1 histone deacetylase complex, may be involved in gene silencing via covalent chromatin modification. (PhosphoSite®, Cell Signaling Technology (Danvers, Mass.), Human PSD™, Biobase Corporation, (Beverly, Mass.)).

Lyn (P07948), phosphorylated at Y315, Y459, Y305, Y500, is among the proteins listed in this patent. Lyn, Lyn protein tyrosine kinase, non-receptor tyrosine kinase, plays a role in cytokine- and IgE-mediated signaling, cell adhesion, apoptosis, platelet activation and inflammatory responses; decreased activity inhibits neoplastic cell transformation. This protein has potential diagnostic and/or therapeutic implications based on the following findings. Induced stimulation of the protein-tyrosine kinase activity of LYN may prevent decreased cell cycle arrest associated with B-cell lymphoma (Proc Natl Acad Sci USA 91: 4048-52 (1994)). Induced stimulation of the protein kinase regulator activity of LYN may prevent abnormal regulation of progression through cell cycle associated with myeloid leukemia (Biochemistry 34: 1058-63 (1995)). Induced stimulation of the protein kinase binding of LYN may prevent abnormal regulation of progression through cell cycle associated with myeloid leukemia (Biochemistry Usa 34: 1058-63 (1995)). Induced stimulation of the protein kinase binding of LYN may prevent abnormal regulation of progression through cell cycle associated with myeloid leukemia (Biochemistry 34: 1058-63 (1995)). Decreased expression of LYN protein may prevent increased cell proliferation associated with myeloid leukemia (Leukemia 13: 855-61 (1999)). Induced stimulation of the protein-tyrosine kinase activity of LYN may prevent abnormal regulation of progression through cell cycle associated with myeloid leukemia (Biochemistry Usa 34: 1058-63 (1995)). Induced stimulation of the protein-tyrosine kinase activity of LYN may cause increased regulation of protein kinase activity associated with myeloid leukemia (Biochemistry 34: 1058-63 (1995)). Induced stimulation of the protein-tyrosine kinase activity of LYN may prevent abnormal regulation of progression through cell cycle associated with myeloid leukemia (Biochemistry 34: 1058-63 (1995)). Decreased expression of LYN protein may prevent neoplasm invasiveness associated with breast neoplasms (J Biol Chem 276: 33711-20 (2001)). Increased expression of LYN protein may cause decreased response to drug associated with chronic myeloid leukemia (Blood 101: 690-8 (2003)). Abnormal protein-tyrosine kinase activity of LYN may prevent Burkitt Lymphoma (Glycobiology 10: 413-9 (2000)). Induced stimulation of the protein-tyrosine kinase activity of LYN may cause increased regulation of protein kinase activity associated with myeloid leukemia (Biochemistry Usa 34: 1058-63 (1995)). Increased protein-tyrosine kinase activity of LYN may cause abnormal cytokine and chemokine mediated signaling pathway associated with chronic myeloid leukemia (J Exp Med 196: 667-78 (2002)). Decreased phosphorylation of LYN may prevent increased cell proliferation associated with chronic myeloid leukemia (Cancer Res 63: 375-81 (2003)). Increased expression of LYN protein correlates with squamous cell carcinoma (JBC 278: 31574-83 (2003)). Induced stimulation of the protein-tyrosine kinase activity of LYN may cause increased anti-apoptosis associated with colonic neoplasms (Cancer Res 61: 5275-83 (2001)). Decreased expression of LYN protein may prevent neoplastic cell transformation associated with breast neoplasms (JBC 276: 33711-20 (2001)). Induced inhibition of the protein-tyrosine kinase activity of LYN may prevent increased cell proliferation associated with myeloid leukemia (Leukemia 13: 855-61 (1999)). Increased expression of LYN protein correlates with disease progression associated with chronic myeloid leukemia (Blood 101: 690-8 (2003)). Induced inhibition of the protein-tyrosine kinase activity of LYN may prevent increased anti-apoptosis associated with B-cell lymphoma (PNAS 92: 9575-9 (1995)). Decreased expression of LYN protein may prevent neoplastic cell transformation associated with breast neoplasms (J Biol Chem 276: 33711-20 (2001)). Decreased expression of LYN protein may prevent neoplasm invasiveness associated with breast neoplasms (JBC 276: 33711-20 (2001)). Decreased phosphorylation of LYN may prevent abnormal protein kinase cascade associated with chronic myeloid leukemia (Cancer Res 63: 375-81 (2003)). Induced inhibition of the protein-tyrosine kinase activity of LYN may prevent increased anti-apoptosis associated with B-cell lymphoma (Proc Natl Acad Sci USA 92: 9575-9 (1995)). Induced inhibition of the protein-tyrosine kinase activity of LYN may prevent increased anti-apoptosis associated with B-cell lymphoma (Proc Natl Acad Sci USA 92: 9575-9 (1995)). Increased protein-tyrosine kinase activity of LYN may cause abnormal G-protein coupled receptor protein signaling pathway associated with chronic myeloid leukemia (J Exp Med 196: 667-78 (2002)). Induced stimulation of the protein-tyrosine kinase activity of LYN may prevent decreased cell cycle arrest associated with B-cell lymphoma (Proc Natl Acad Sci USA 91: 4048-52 (1994)). Induced stimulation of the protein kinase regulator activity of LYN may prevent abnormal regulation of progression through cell cycle associated with myeloid leukemia (Biochemistry Usa 34: 1058-63 (1995)). Increased expression of LYN protein correlates with squamous cell carcinoma associated with head and neck neoplasms (JBC 278: 31574-83 (2003)). Increased expression of LYN protein correlates with squamous cell carcinoma (J Biol Chem 278: 31574-83 (2003)). Increased expression of LYN protein correlates with squamous cell carcinoma associated with head and neck neoplasms (J Biol Chem 278: 31574-83 (2003)). Induced stimulation of the protein-tyrosine kinase activity of LYN may prevent decreased cell cycle arrest associated with B-cell lymphoma (PNAS 91: 4048-52 (1994)). Increased protein-tyrosine kinase activity of LYN may cause abnormal chemotaxis associated with chronic myeloid leukemia (J Exp Med 196: 667-78 (2002)). Induced stimulation of the protein-tyrosine kinase activity of LYN may cause drug-resistant form of colonic neoplasms (Cancer Res 61: 5275-83 (2001)). (PhosphoSite®, Cell Signaling Technology (Danvers, Mass.), Human PSD™, Biobase Corporation, (Beverly, Mass.)).

MAPKAPK3 (Q16644), phosphorylated at Y76, is among the proteins listed in this patent. MAPKAPK3, Mitogen-activated protein kinase-activated protein kinase 3, putative serine/threonine kinase activated by hyperosmotic stress or tumor necrosis factor (TNF) via phosphorylation by CSBP1/CSBP2 (MAPK14), phosphorylates the small heat shock protein HSP27. This protein has potential diagnostic and/or therapeutic implications based on the following findings. MAPKAPK3 map position may correlate with small-cell tumors associated with lung neoplasms (Mol Cell Biol. 16: 868-76 (1996)). MAPKAPK3 map position may correlate with small-cell tumors associated with lung neoplasms (MCB 16: 868-76 (1996)). MAPKAPK3 map position may correlate with small-cell tumors associated with lung neoplasms (Mol Cell Biol 16: 868-76 (1996)). MAPKAPK3 map position may correlate with small-cell tumors associated with lung neoplasms (Mol. Cell Biol 16: 868-76 (1996)). MAPKAPK3 map position may correlate with small-cell tumors associated with lung neoplasms (Mol. Cell. Biol. 16: 868-76 (1996)). (PhosphoSite®, Cell Signaling Technology (Danvers, Mass.), Human PSD™, Biobase Corporation, (Beverly, Mass.)).

MCM7 (P33993), phosphorylated at Y492, is among the proteins listed in this patent. MCM7, MCM7 minichromosome maintenance deficient 7, part of a ssDNA- and ATP-dependent helicase complex involved in DNA replication, increased expression is associated with prostate and hypopharyngeal cancers, cervical carcinoma and MYCN-amplified neuroblastoma. (PhosphoSite®, Cell Signaling Technology (Danvers, Mass.), Human PSD™, Biobase Corporation, (Beverly, Mass.)).

MKK6 (P52564), phosphorylated at Y64, is among the proteins listed in this patent. MKK6, Mitogen-activated protein kinase kinase 6, a threonine-tyrosine kinase involved in signal transduction, phosphorylates the MAP kinase p38, involved in promoting cell cycle arrest and protection from apoptosis in response to a variety of insults. This protein has potential diagnostic and/or therapeutic implications based on the following findings. Increased expression of MAP2K6 in neurons may cause abnormal activation of MAPK activity associated with Alzheimer disease (J Neurochem 79: 311-8 (2001)). Mislocalization of MAP2K6 protein may cause abnormal activation of MAPK activity associated with Alzheimer disease (J Neurochem 79: 311-8 (2001)). Increased phosphorylation of MAP2K6 correlates with Alzheimer disease (J Neurochem 79: 311-8 (2001)) (PhosphoSite®, Cell Signaling Technology (Danvers, Mass.), Human PSD™, Biobase Corporation, (Beverly, Mass.)).

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. As used herein, unless specifically indicated otherwise, the word “or” is used in the “inclusive” sense of “and/or” and not the “exclusive” sense of “either/or.”

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2, can be 0, 1 or 2 for variables which are inherently discrete, and can be 0.0, 0.1, 0.01, 0.001, or any other real value for variables which are inherently continuous.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

“Antibody” or “antibodies” refers to all classes of immunoglobulins, including IgG, IgM, IgA, IgD, and IgE, including whole antibodies and any antigen biding fragment thereof (e.g., F_{ab}) or single chains thereof, including chimeric, polyclonal, and monoclonal antibodies. Antibodies are antigen-specific protein molecules produced by lymphocytes of the B cell lineage. Following antigenic stimulation, B cells that have surface immunoglobulin receptors that bind the antigen clonally expand, and the binding affinity for the antigen increases through a process called affinity maturation. The B cells further differentiate into plasma cells, which secrete large quantities of antibodies in to the serum. While the physiological role of antibodies is to protect the host animal by specifically binding and eliminating microbes and microbial pathogens from the body, large amounts of antibodies are also induced by intentional immunization to produce specific antibodies that are used extensively in many biomedical and therapeutic applications.

Antibody molecules are shaped somewhat like the letter “Y”, and consist of 4 protein chains, two heavy (H) and two light (L) chains. Antibodies possess two distinct and spatially separate functional features. The ends of each of the two arms of the “Y” contain the variable regions (variable heavy (V(H)) and variable light ( V(L)) regions), which form two identical antigen-binding sites. The variable regions undergo a process of “affinity maturation” during the immune response, leading to a rapid divergence of amino acids within these variable regions. The other end of the antibody molecule, the stem of the “Y”, contains only the two heavy constant (CH) regions, interacts with effector cells to determine the effector functions of the antibody. There are five different CH region genes that encode the five different classes of immunoglobulins: IgM, IgD, IgG, IgA and IgE. These constant regions, by interacting with different effector cells and molecules, determine the immunoglobulin molecule's biological function and biological response.

Each V(H) and V(L) region contains three subregions called complementarity determining regions. These include CDR1-3 of the V(H) domain and CDR1-3 of the V(L) domain. These six CDRs generally form the antigen binding surface, and include those residues that hypermutate during the affinity maturation phase of the immune response. The CDR3 of the V(H) domain seems to play a dominant role in generating diversity oof both the B cell antigen receptor (BCR) and the T cell antigen receptor systems (Xu et al., Immunity 13:37-45(2000)).

The term “antibody” or “antibodies” refers to all classes of polyclonal or monoclonal immunoglobulins, including IgG, IgM, IgA, IgD, and IgE, including whole antibodies and any antigen binding fragment thereof. This includes any combination of immunoglobulin domains or chains that contains a variable region (V(H) or V(L)) that retains the ability to bind the immunogen. Such fragments include F(ab)₂ fragments (V(H)-C(H1), V(L)-C(L))₂; monovalent Fab fragments (V(H)-C(H1), V(L)-C(L)); Fv fragment (V(H)-V(L); single-chain Fv fragments (Kobayashi et al., Steroids July;67(8):733-42 (2002).

Monoclonal antibodies refer to clonal antibodies produced from fusions between immunized murine, rabbit, human, or other vertebrate species, and produced by classical fusion technology Kohler G, Milstein C. Continuous cultures of fused cells secreting antibody of predefined specificity. Nature Aug. 7, 1975;256(5517):495-7 or by alternative methods which may isolate clones of immunoglobulin secreting cells from transformed plasma cells.

When used with respect to an antibody's binding to one phospho-form of a sequence, the expression “does not bind” means that a phospho-specific antibody either does not apparently bind to the non-phospho form of the antigen as ascertained in commonly used experimental detection systems (Western blotting, IHC, Immunofluorescence, etc.). One of skill in the art will appreciate that the expression may be applicable in those instances when (1) a phospho-specific antibody either does not apparently bind to the non-phospho form of the antigen as ascertained in commonly used experimental detection systems (Western blotting, IHC, Immunofluorescence, etc.); (2) where there is some reactivity with the surrounding amino acid sequence, but that the phosphorylated residue is an immunodominant feature of the reaction. In cases such as these, there is an apparent difference in affinities for the two sequences. Dilutional analyses of such antibodies indicates that the antibodies apparent affinity for the phosphorylated form is at least 10-100 fold higher than for the non-phosphorylated form; or where (3) the phospho-specific antibody reacts no more than an appropriate control antibody would react under identical experimental conditions. A control antibody preparation might be, for instance, purified immunoglobulin from a pre-immune animal of the same species, an isotype- and species-matched monoclonal antibody. Tests using control antibodies to demonstrate specificity are recognized by one of skill in the art as appropriate and definitive.

“Target signaling protein/polypeptide” means any protein (or polypeptide derived therefrom) enumerated in Column A of Table 1/FIG. 2, which is disclosed herein as being phosphorylated in one or more cell line(s). Target signaling protein(s)/polypeptide(s) may be tyrosine kinases, such as TTN or BCR, or serine/threonine kinases, or direct substrates of such kinases, or may be indirect substrates downstream of such kinases in signaling pathways. Target signaling protein/polypeptide where elucidated in leukemia cell lines, however one of skill in the art will appreciate that a target signaling protein/polypeptide may also be phosphorylated in other cell lines (non-leukemic) harboring activated kinase activity.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with polypeptide, and includes protein fragments and domains as well as whole protein.

“Phosphorylatable amino acid” means any amino acid that is capable of being modified by addition of a phosphate group, and includes both forms of such amino acid.

“Phosphorylatable peptide sequence” means a peptide sequence comprising a phosphorylatable amino acid.

“Phosphorylation site-specific antibody” means an antibody that specifically binds a phosphorylatable peptide sequence/epitope only when phosphorylated, or only when not phosphorylated, respectively. The term is used interchangeably with “phospho-specific” antibody.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

A. Identification of Phosphorylation Sites. The Target signaling protein/polypeptide phosphorylation sites disclosed herein and listed in Table 1/FIG. 2 were discovered by employing the modified peptide isolation and characterization techniques described in U.S. Pat. No. 7,198,896 using cellular extracts from the following human cancer cell lines, tissues and patient samples: 01364548-cll, 223-CLL, 293T, 3T3 TrkB, 3T3-Src, 3T3-TrkA, 3T3-wt, 577, A172, AML-4833, AML-6246, AML-6735, AML-7592, BaF3-10ZF, BaF3-4ZF, BaF3-APR, BaF3-FLT3 (D842V), BaF3-FLT3(D842Y), BaF3-FLT3(K663Q), BaF3-FLT3(WT), BaF3-FLT3/ITD, BaF3-PRTK, BaF3-TDII, BaF3-Tel/FGFR3, Baf3, Baf3-V617F -jak2, Baf3/E255K, Baf3/H396P, Baf3/Jak2(IL-3 dep), Baf3/M351T, Baf3/T3151, Baf3/TpoR, Baf3/TpoR-Y98F, Baf3/Tyk2, Baf3/V617F-jak2 (IL-3), Baf3Y253F, Baf3/cc-TpoR-IV, Baf3/p201wt, CHRF, CI-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC1937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOPT-K1, KY821, Karpas 299, Karpas-1106p, M-07e, M01043, M059K, MC-116, MCF-10A (Y561F), MCF-10A(Y969F), MDA-MB-453, MDA-MB-468, MEC-2, MKPL-1, ML-1, MO-91, MOLT15, MV4-11, Me-F2, Molm 14, Monomac 6, NCI-N87, Nomo-1, OCI-M1, OCI-ly4, OCI-ly8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, h1144b, h1152b, lung tumor T26, lung tumor T57, normal human lung, pancreatic xenograft, patient 1, rat brain, sw480. The isolation and identification of phosphopeptides from these cell lines, using an immobilized general phosphotyrosine-specific antibody, or an antibody recognizing the phosphorylated motif PXpSP is described in detail in Example 1 below. In addition to the protein phosphorylation sites (tyrosine) described herein, many known phosphorylation sites were also identified (not described herein). The immunoaffinity/mass spectrometric technique described in the '896 Patent (the ∂IAP” method)—and employed as described in detail in the Examples—is briefly summarized below.

The IAP method employed generally comprises the following steps: (a) a proteinaceous preparation (e.g. a digested cell extract) comprising phosphopeptides from two or more different proteins is obtained from an organism; (b) the preparation is contacted with at least one immobilized general phosphotyrosine-specific antibody; (c) at least one phosphopeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g., Sequest) may be utilized to substantially match the spectra obtained for the isolated, modified peptide during the characterization of step (d) with the spectra for a known peptide sequence. A quantification step employing, e.g., SILAC or AQUA, may also be employed to quantify isolated peptides in order to compare peptide levels in a sample to a baseline.

In the IAP method as employed herein, a general phosphotyrosine-specific monoclonal antibody (commercially available from Cell Signaling Technology, Inc., Beverly, Mass., Cat. #9411 (p-Tyr-100)) was used in the immunoaffinity step to isolate the widest possible number of phospho-tyrosine containing peptides from the cell extracts.

Extracts from the following human cancer cell lines, tissues and patient samples were employed: 01364548-cll, 223-CLL, 293T, 3T3 TrkB, 3T3-Src, 3T3-TrkA, 3T3-wt, 577, A172, AML-4833, AML-6246, AML-6735, AML-7592, BaF3-10ZF, BaF3-4ZF, BaF3-APR, BaF3-FLT3(D842V), BaF3-FLT3(D842Y), BaF3-FLT3(K663Q), BaF3-FLT3(WT), BaF3-FLT3/TTD, BaF3-PRTK, BaF3-TDII, BaF3-Tel/FGFR3, Baf3, Baf3-V617F -jak2, Baf3/E255K, Baf3/H396P, Baf3/Jak2(IL-3 dep), Baf3/M351T, Baf3/T3151, Baf3/TpoR, Baf3/TpoR-Y98F, Baf3/Tyk2, Baf3/V617Fjak2 (IL-3), Baf3/Y253F, Baf3/cc-TpoR-IV, Baf3/p210wt, CHRF, C1-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC]937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOPT-K1, KY821, Karpas 299, Karpas-1106p, M-07e, M01043, M059K, MC-116, MCF-10A (Y561F), MCF-10A(Y969F), MDA-MB-453, MDA-MB468, MEC-2, MKPL-1, ML-1, MO-91, MOLT15, MV4-11, Me-F2, Molm 14, Monomac 6, NCI-N87, Nomo-1, OCI-M1, OCI-ly4, OCI-ly8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, h1144b, h1152b, lung tumor T26, lung tumor T57, normal human lung, pancreatic xenograft, patient 1, rat brain and sw480.

As described in more detail in the Examples, lysates were prepared from these cells and digested with trypsin after treatment with DTT and iodoacetamide to redue and alkylate cysteine residues. Before the immunoaffinity step, peptides were pre-fractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in MOPS IP buffer and treated with phosphotyrosine (P-Tyr-100, CST #9411) immobilized on protein G-Sepharose. Immunoaffinity-purified peptides were eluted with 0.1% TFA and a portion of this fraction was concentrated with Stage or Zip tips and analyzed by LC-MS/MS, using either a LCQ or ThermoFinnigan LTQ ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

This revealed the tyrosine phosphorylation sites in signaling pathways affected by kinase activation or active in leukemia cells. The identified phosphorylation sites and their parent proteins are enumerated in Table 1/FIG. 2. The tyrosine at which phosphorylation occurs is provided in Column D, and the peptide sequence encompassing the phosphorylatable tyrosine residue at the site is provided in Column E. If a phosphorylated tyrosine was found in mouse, the orthologous site in human was identified using either Homologene or BLAST at NCBI; the sequence reported in column E is the phosphorylation site flanked by 7 amino acids on each side. FIG. 2 also shows the particular type of leukemic disease (see Column G) and cell line(s) (see Column F) in which a particular phosphorylation site was discovered.

As a result of the discovery of these phosphorylation sites, phospho-specific antibodies and AQUA peptides for the detection of and quantification of these sites and their parent proteins may now be produced by standard methods, as described below. These new reagents will prove highly useful in, e.g., studying the signaling pathways and events underlying the progression of leukemias and the identification of new biomarkers and targets for diagnosis and treatment of such diseases in a mammal.

The methods of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

B. Antibodies and Cell Lines. Isolated phosphorylation site-specific antibodies that specifically bind a target signaling protein/polypeptide disclosed in Column A of Table 1 only when phosphorylated (or only when not phosphorylated) at the corresponding amino acid and phosphorylation site listed in Columns D and E of Table 1/FIG. 2 may be produced by standard antibody production methods, such as anti-peptide antibody methods, using the phosphorylation site sequence information provided in Column E of Table 1. The ITSN2 adaptor/scaffold protein phosphorylation site (tyrosine 261) (see Row 24 of Table 1/FIG. 2) is presently disclosed. Thus, an antibody that specifically binds this novel ITSN2 adaptor/scaffold site can now be produced, e.g. by immunizing an animal with a peptide antigen comprising all or part of the amino acid sequence encompassing the respective phosphorylated residue (e.g., a peptide antigen comprising the sequence set forth in Row 24, Column E, of Table 1, SEQ ID NO: 23, respectively) (which encompasses the phosphorylated tyrosine at position 261 in ITSN2, to produce an antibody that only binds ITSN2 adaptor/scaffold when phosphorylated at that site.

Polyclonal antibodies of the invention may be produced according to standard techniques by immunizing a suitable animal (e.g., rabbit, goat, etc.) with a peptide antigen corresponding to the phosphorylation site of interest (i.e., a phosphorylation site enumerated in Column E of Table 1, which comprises the corresponding phosphorylatable amino acid listed in Column D of Table 1), collecting immune serum from the animal, and separating the polyclonal antibodies from the immune serum, in accordance with known procedures. For example, a peptide antigen corresponding to all or part of the novel HSPG2 adhesion or extra-celluar matrix protein phosphorylation site disclosed herein (SEQ ID NO: 36=YNVRyELAR, encompassing phosphorylated tyrosine 620 (see Row 37 of Table 1)) may be employed to produce antibodies that only bind Crkl when phosphorylated at Tyr 620. Similarly, a peptide comprising all or part of any one of the phosphorylation site sequences provided in Column E of Table 1 may employed as an antigen to produce an antibody that only binds the corresponding protein listed in Column A of Table 1 when phosphorylated (or when not phosphorylated) at the corresponding residue listed in Column D. If an antibody that only binds the protein when phosphorylated at the disclosed site is desired, the peptide antigen includes the phosphorylated form of the amino acid. Conversely, if an antibody that only binds the protein when not phosphorylated at the disclosed site is desired, the peptide antigen includes the non-phosphorylated form of the amino acid.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

It will be appreciated by those of skill in the art that longer or shorter phosphopeptide antigens may be employed. See Id. For example, a peptide antigen may comprise the full sequence disclosed in Column E of Table 1/FIG. 2, or it may comprise additional amino acids flanking such disclosed sequence, or may comprise of only a portion of the disclosed sequence immediately flanking the phosphorylatable amino acid (indicated in Column E by lowercase “y”). Typically, a desirable peptide antigen will comprise four or more amino acids flanking each side of the phosphorylatable amino acid and encompassing it. Polyclonal antibodies produced as described herein may be screened as further described below.

Monoclonal antibodies of the invention may be produced in a hybridoma cell line according to the well-known technique of Kohler and Milstein. See Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal F_{ab} fragments may also be produced in Escherichia coli by recombinant techniques known to those skilled in the art. See, e.g., W. Huse, Science 246: 1275-81 (1989); Mullinax et al., Proc. Nat'l. Acad. Sci. 87: 8095 (1990). If monoclonal antibodies of one isotype are preferable for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

An epitope of a phosphorylation-site specific antibody of the invention is a peptide fragment consisting essentially of about 8 to 17 amino acids including the phosphorylatable tyrosine, wherein about 3 to 8 amino acids are positioned on each side of the phosphorylatable tyrosine (for example, the HIVEP2 tyrosine 1788 phosphorylation site sequence disclosed in Row 64, Column E of Table 1), and antibodies of the invention thus specifically bind a target signal protein/polypepetide comprising such epitopic sequence. Epitopes bound by the antibodies of the invention comprise all or part of a phosphorylatable site sequence listed in Column E of Table 1, including the phosphorylatable amino acid.

Included in the scope of the invention are equivalent non-antibody molecules, such as protein binding domains or nucleic acid aptamers, which bind, in a phospho-specific manner, to essentially the same phosphorylatable epitope to which the phospho-specific antibodies of the invention bind. See, e.g., Neuberger et al., Nature 312: 604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 or U.S. Pat. No. 4,816,567. The antibodies may also be chemically constructed by specific antibodies made according to the method disclosed in U.S. Pat. No. 4,676,980.

The invention also provides immortalized cell lines that produce an antibody of the invention. For example, hybridoma clones, constructed as described above, that produce monoclonal antibodies to the protein phosphorylation sites disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., Antibody Engineering Protocols, 1995, Humana Press, Sudhir Paul editor.)

Phosphorylation site-specific antibodies of the invention, whether polyclonal or monoclonal, may be screened for epitope and phospho-specificity according to standard techniques. See, e.g. Czernik et al., Methods in Enzymology, 201: 264-283 (1991). For example, the antibodies may be screened against the phospho and non-phospho peptide library by ELISA to ensure specificity for both the desired antigen (i.e., that epitope including a phosphorylation site sequence enumerated in Column E of Table 1) and for reactivity only with the phosphorylated (or non-phosphorylated) form of the antigen. Peptide competition assays may be carried out to confirm lack of reactivity with other phospho-epitopes on the given Target Signal Protein/Polypepetide. The antibodies may also be tested by Western blotting against cell preparations containing the signaling protein, e.g. cell lines over-expressing the target protein, to confirm reactivity with the desired phosphorylated epitope/target.

In an exemplary embodiment, phage display libraries containing more than 10¹⁰ phage clones are used for high-throughput production of monoclonal antibodies that target post-translational modification sites (e.g., phosphorylation sites) and, for validation and quality control, high-throughput immunohistochemistry is utilized to screen the efficacy of these antibodies. Western blots, protein microarrays and flow cytometry can also be used in high-throughput screening of phosphorylation site-specific polyclonal or monoclonal antibodies of the present invention. See, e.g., Blow N., Nature, 447: 741-743 (2007).

Specificity against the desired phosphorylated epitope may also be examined by constructing mutants lacking phosphorylatable residues at positions outside the desired epitope that are known to be phosphorylated, or by mutating the desired phospho-epitope and confirming lack of reactivity. Phosphorylation-site specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify sites highly homologous to the target signaling protein/polypeptide epitope for which the antibody of the invention is specific.

In certain cases, polyclonal antisera may exhibit some undesirable general cross-reactivity to phosphotyrosine or phosphoserine itself, which may be removed by further purification of antisera, e.g., over a phosphotyramine column. Antibodies of the invention specifically bind their target protein (i.e., a protein listed in Column A of Table 1) only when phosphorylated (or only when not phosphorylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to evaluate phosphorylation and activation status in diseased tissue. IHC may be carried out according to well-known techniques. See, e.g., Antibodies: A Laboratory Manual, Chapter 10, Harlow & Lane Eds., Cold Spring Harbor Laboratory (1 988). Briefly, paraffin-embedded tissue (e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary phosphorylation-site specific antibody of the invention (which detects a target Signal Protein/Polypepetide enumerated in Table 1), washed and labeled with a fluorescent-labeled secondary antibody. Additional fluorochrome-conjugated marker antibodies (e.g., CD45, CD34) may also be added at this time to aid in the subsequent identification of specific hematopoietic cell types. The cells would then be analyzed on a flow cytometer (e.g., a Beckman Coulter FC500) according to the specific protocols of the instrument used.

Antibodies of the invention may also be advantageously conjugated to fluorescent dyes (e.g., Alexa488, PE) for use in multi-parametric analyses along with other signal transduction (phospho-CrkL, phospho-Erk 1/2) and/or cell marker (CD34) antibodies.

Phosphorylation-site specific antibodies of the invention specifically bind to a target signaling protein/polypeptide only when phosphorylated at a disclosed site, but are not limited only to binding the human species, per se. The invention includes antibodies that also bind conserved and highly homologous or identical phosphorylation sites in respective target signaling protein/polypeptide from other species (e.g., mouse, rat, monkey, yeast), in addition to binding the human phosphorylation site. Highly homologous or identical sites conserved in other species can readily be identified by standard sequence comparisons, such as using BLAST, with the human target signaling protein/polypeptide phosphorylation sites disclosed herein.

C. Heavy-Isotope Labeled Peptides (AQUA Peptides). The phosphorylation sites disclosed herein now enable the production of corresponding heavy-isotope labeled peptides for the absolute quantification of such signaling proteins (both phosphorylated and not phosphorylated at a disclosed site) in biological samples. The production and use of AQUA peptides for the absolute quantification of proteins (AQUA) in complex mixtures has been described. See WO/03016861, Gerber et al., Proc. Natl. Acad. Sci. U.S.A. 100: 6940-5 (2003).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al., supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g., trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g., 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known phosphorylation site sequence previously identified by the IAP-LC-MS/MS method within a target protein. One AQUA peptide incorporating the phosphorylated form of the particular residue within the site may be developed, and a second AQUA peptide incorporating the non-phosphorylated form of the residue developed. In this way, the two standards may be used to detect and quantify both the phosphorylated and non-phosphorylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g., trypsin, hepsin), metallo proteases (e.g., PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. A workable range is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: the mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are suitable labels. Pairs of peptide internal standards that incorporate a different isotope label may also be prepared. Amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts may be employed. Generally, the sample has at least 0.01 mg of protein, typically a concentration of 0.1-10 mg/mL, and may be adjusted to a desired buffer concentration and pH.

A known amount of a labeled peptide internal standard, preferably about femtomoles, corresponding to a target protein to be detected/quantified is then added to a biological sample, such as a cell lysate. The spiked sample is then digested with one or more protease(s) for a suitable time period to allow digestion. A separation is then performed (e.g., by HPLC, reverse-phase HPLC, capillary electrophoresis, ion exchange chromatography, etc.) to isolate the labeled internal standard and its corresponding target peptide from other peptides in the sample. Microcapillary LC is a method contemplated.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the phosphorylation sites disclosed herein. Peptide standards for a given phosphorylation site (e.g., the tyrosine 724 in HADHA—see Row 116 of Table 1) may be produced for both the phosphorylated and non-phosphorylated forms of the site (e.g., see HADHA site sequence in Column E, Row 116 of Table 1 (SEQ ID NO: 115) and such standards employed in the AQUA methodology to detect and quantify both forms of such phosphorylation site in a biological sample.

AQUA peptides of the invention may comprise all, or part of, a phosphorylation site peptide sequence disclosed herein (see Column E of Table 1/FIG. 2). In an embodiment, an AQUA peptide of the invention comprises a phosphorylation site sequence disclosed herein in Table 1/FIG. 2. For example, an AQUA peptide of the invention for detection/quantification of HIP14 Enzyme protein when phosphorylated at tyrosine Y67 may comprise the sequence ATQyGIYER (y=phosphotyrosine), which comprises phosphorylatable tyrosine 67 (see Row 123, Column E; (SEQ ID NO: 122)). Heavy-isotope labeled equivalents of the peptides enumerated in Table 1/FIG. 2 (both in phosphorylated and unphosphorylated form) can be readily synthesized and their unique MS and LC-SRM signature determined, so that the peptides are validated as AQUA peptides and ready for use in quantification experiments.

The phosphorylation site peptide sequences disclosed herein (see Column E of Table 1/FIG. 2) are well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in phosphorylated and unphosphorylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the phosphorylation sites disclosed in Table 1/FIG. 2 (see Column E) and/or their corresponding parent proteins/polypeptides (see Column A). A phosphopeptide sequence comprising any of the phosphorylation sequences listed in Table 1 may be considered an AQUA peptide of the invention. For example, an AQUA peptide comprising the sequence TNHHSCLySAVK (SEQ ID NO: 119) (where y may be either phosphotyrosine or tyrosine, and where V=labeled valine (e.g., ¹⁴C)) is provided for the quantification of phosphorylated (or non-phosphorylated) helicase B (Tyr721) in a biological sample (see Row 120 of Table 1, tyrosine 721 being the phosphorylatable residue within the site). It will be appreciated that a larger AQUA peptide comprising a disclosed phosphorylation site sequence (and additional residues downstream or upstream of it) may also be constructed. Similarly, a smaller AQUA peptide comprising less than all of the residues of a disclosed phosphorylation site sequence (but still comprising the phosphorylatable residue enumerated in Column D of Table 1/FIG. 2) may alternatively be constructed. Such larger or shorter AQUA peptides are within the scope of the present invention, and the selection and production of AQUA peptides may be carried out as described above (see Gygi et al., Gerber et al., supra.).

Certain subsets of AQUA peptides provided by the invention are described above (corresponding to particular protein types/groups in Table 1, for example, tyrosine protein kinases or adaptor/scaffold proteins). Example 4 is provided to further illustrate the construction and use, by standard methods described above, of exemplary AQUA peptides provided by the invention. For example, the above-described AQUA peptides corresponding to both the phosphorylated and non-phosphorylated forms of the disclosed G-alpha-s G-protein or regulator protein tyrosine 311 phosphorylation site (see Row 144 of Table 1/FIG. 2) may be used to quantify the amount of phosphorylated claspin (Tyr 311) in a biological sample, e.g., a tumor cell sample (or a sample before or after treatment with a test drug).

AQUA peptides of the invention may also be employed within a kit that comprises one or multiple AQUA peptide(s) provided herein (for the quantification of a target signaling protein/polypeptide disclosed in Table 1/FIG. 2), and, optionally, a second detecting reagent conjugated to a detectable group. For example, a kit may include AQUA peptides for both the phosphorylated and non-phosphorylated form of a phosphorylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be useful in the further study of signal transduction anomalies associated with diseases such as for example cancer, including leukemias, and in identifying diagnostic/bio-markers of these diseases, new potential drug targets, and/or in monitoring the effects of test compounds on target Signaling Proteins/Polypeptides and pathways.

D. Immunoassay Formats. Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a phosphorylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a phosphorylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No.4,727,022; U.S. Pat. No. 4,659,678; U.S. Pat. No. 4,376,110. Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a target signaling protein/polypeptide is detectable compared to background.

Phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry (FC) assay to determine the activation/phosphorylation status of a target signaling protein/polypeptide in patients before, during, and after treatment with a drug targeted at inhibiting phosphorylation of such a protein at the phosphorylation site disclosed herein. For example, bone marrow cells or peripheral blood cells from patients may be analyzed by flow cytometry for target signaling protein/polypeptide phosphorylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% para-formaldehyde for 10 minutes at 37° C. followed by permeabilization in 90% methanol for 30 minutes on ice. Cells may then be stained with the primary antibody (a phospho-specific antibody of the invention), washed and labeled with a fluorescent-labeled secondary antibody. Alternatively, the cells may be stained with a fluorescent-labeled primary antibody. The cells would then be analyzed on a flow cytometer (e.g., a Beckman Coulter EPICS-XL) according to the specific protocols of the instrument used. Such an analysis would identify the presence of activated target Signaling Protein(s)/Polypeptide(s) in the malignant cells and reveal the drug response on the targeted protein.

Alternatively, antibodies of the invention may be employed in immunohistochemical (IHC) staining to detect differences in signal transduction or protein activity using normal and diseased tissues. IHC may be carried out according to well-known techniques. See, e.g., ANTIBODIES: A LABORATORY MANUAL, supra. Briefly, paraffin-embedded tissue (e.g., tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody array formats, such as reversed-phase array applications (see, e.g., Paweletz et al., Oncogene 20(16): 1981-89 (2001)). Accordingly, in another embodiment, the invention provides a method for the multiplex detection of phosphorylation in a biological sample, the method comprising utilizing two or more antibodies or AQUA peptides of the invention to detect the presence of two or more phosphorylated proteins enumerated in Column A of Table 1/FIG. 2. In an embodiment, two to five antibodies or AQUA peptides of the invention are employed in the method. In another embodiment, six to ten antibodies or AQUA peptides of the invention are employed, while in another embodiment eleven to twenty such reagents are employed.

Antibodies and/or AQUA peptides of the invention may also be employed within a kit that comprises at least one phosphorylation site-specific antibody or AQUA peptide of the invention (which binds to or detects a target signaling protein/polypeptide disclosed in Table 1/FIG. 2), and, optionally, a second antibody conjugated to a detectable group. In some embodies, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the description, numerous specific details are set forth in order to provide a thorough understanding of the present invention. The present invention may be practiced without some or all of these specific details. In other instances, well known process operations have not been described in detail, in order not to unnecessarily obscure the present invention.

The following examples are intended to further illustrate certain embodiments of the invention and are not limiting in nature. Those skilled in the art will recognize, or be able to ascertain, using no more than routine experimentation, numerous equivalents to the specific substances and procedures described herein.

Any suitable materials and/or methods known to those of skill can be utilized in carrying out the present invention. However, materials and methods are described. Materials, reagents and the like to which reference is made in the following description and examples are obtainable from commercial sources, unless otherwise noted.

### Example 1

### Isolation of Phosphotyrosine-Containing Peptides from Extracts of Cancer Cell Lines and Identification of Novel Phosphorylation Sites

IAP isolation techniques were employed to identify phosphotyrosine containing peptides in cell extracts from the following human cancer cell lines, tissues and patient cell lines: 01364548-cll, 223-CLL, 293T, 3T3 TrkB, 3T3-Src, 3T3-TrkA, 3T3-wt, 577, A172, AML-4833, AML-6246, AML-6735, AML-7592, BaF3-10ZF, BaF3-4ZF, BaF3-APR, BaF3-FLT3(D842V), BaF3-FL,T3(D842Y), BaF3-FLT3(K663Q), BaF3-FLT3(WT), BaF3-FLT3/1TD, BaF3-PRTK, BaF3-TDII, BaF3-Te1/FGFR3, Baf3, Baf3-V617F jak2, Baf3/E255K, Baf3/H396P, Baf3/Jak2(IL-3 dep), Baf3/M35 IT, Baf3/T315I, Baf3/TpoR, Baf3/TpoR-Y98F, Baf3/Tyk2, Baf3/V617Fjak2 (IL-3), Baf3/NY253F, Baf3/cc-TpoR-IV, Baf3/p210wt, CHRF, CI-1, CMK, CTV-1, DMS 53, DND41, DU-528, DU145, ELF-153, EOL-1, GDM-1, H1703, H1734, H1793, H1869, H1944, H1993, H2023, H226, H3255, H358, H520, H82, H838, HCC1428, HCC1435, HCC1806, HCC1937, HCC366, HCC827, HCT116, HEL, HL107B, HL117B, HL131A, HL131B, HL133A, HL53B, HL59b, HL60, HL61a, HL61b, HL66B, HL68A, HL75A, HL84A, HL97B, HL98A, HT29, HU-3, HUVEC, Jurkat, K562, KG-1, KG1-A, KMS11, KMS18, KMS27, KOPT-K1, KY821, Karpas 299, Karpas-1106p, M-07e, M01043, M059K, MC-116, MCF-10A (Y561F), MCF-10A(Y969F), MDA-MB-453, MDA-MB-468, MEC-2, MKPL-1, ML-1, MO-91, MOLT15, MV4-11, Me-F2, Molm 14, Monomac 6, NCI-N87, Nomo-1, OCI-M 1, OCI-1y4, OCI-1y8, OCI/AML2, OPM-1, PL21, Pfeiffer, RC-K8, RI-1, SCLC T1, SEM, SK-N-AS, SK-N-MC, SKBR3, SR-786, SU-DHL1, SUP-M2, SUPT-13, SuDHL5, T17, TRE-cll patient, TS, UT-7, VAL, Verona, Verona 1, Verona 4, WSU-NHL, XG2, Z-55, cs001, cs015, cs025, cs041, cs042, gz21, gz68, gz73, gz74, gzB1, h1144b, h1152b, lung tumor T26, lung tumor T57, normal human lung, pancreatic xenograft, patient 1, rat brain and sw480.

Tryptic phosphotyrosine containing peptides were purified and analyzed from extracts of each of the cell lines mentioned above, as follows. Cells were cultured in DMEM medium or RPMI 1640 medium supplemented with 10% fetal bovine serum and penicillin/streptomycin.

Suspension cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM β-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK®-trypsin (Worthington® Biochemcial Corporation, Lakewood, N.J.) was added at 10-20 μg/mL. Digestion was performed for 1-2 days at room temperature.

Trifluoroacetic acid (TFA) was added to protein digests to a final concentration of 1%, precipitate was removed by centrifugation, and digests were loaded onto Sep-Pak® C₁₈ columns (provided by Waters Corporation, Milford, Mass.) equilibrated with 0.1% TFA. A column volume of 0.7-1.0 ml was used per 2×10⁸ cells. Columns were washed with 15 volumes of 0. 1% TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1% TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble material was removed by centrifugation. IAP was performed on each peptide fraction separately. The phosphotyrosine monoclonal antibody P-Tyr-100 (Cell Signaling Technology®, Inc., Danvers, Mass. catalog number 9411) was coupled at 4 mg/ml beads to protein G or protein A agarose (Roche®, Basel, Switzerland), respectively. Immobilized antibody (15 μl, 60 μg) was added as 1:1 slurry in IAP buffer to 1.4 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitirile in 0.1% TFA and combination of all eluates. IAP on this peptide fraction was performed as follows: After lyophilization, peptide was dissolved in 1.4 ml IAP buffer (MOPS pH 7.2, 10 mM sodium phosphate, 50 mM NaCl) and insoluble material was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 40 μl of 0.15% TFA at room temperature for 10 min (eluate 1), followed by a wash of the beads (eluate 2) with 40 μl of 0.15% TFA. Both eluates were combined.

### Analysis by LC-MS/MS Mass Spectrometry.

40 μl or more of IAP eluate were purified by 0.2 μl StageTips (Proxeon, Staermosegaardsvej 6,DK-5230 Odense M, Denmark) or ZipTips® (produced by Millipore®, Billerica Mass.). Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit® capillary column (produced by New Objective, Woburn, Mass.) packed with Michrom Magic Bullets® C18 AQ reversed-phase resin (Michrom Bioresources, Auburn Calif.) using a Famos™ autosampler with an inert sample injection valve (Dionex®, Sunnyvale, Calif.). The column was then developed with a 45-min linear gradient of acetonitrile delivered at 200 nl/min (using an Ultimate® pump, Dionex®, Sunnyvale, Calif.), and tandem mass spectra were collected in a data-dependent manner with an LTQ® (produced by Thermo® Finnigan® San, Jose, Calif.), ion trap mass spectrometer essentially as described by Gygi et al., supra.

### Database Analysis & Assignments.

MS/MS spectra were evaluated using TurboSequest™ in the Sequest® (owned by Thermo® Finnigan® San Jose, Calif.) Browser package (v. 27, rev. 12) supplied as part of BioWorks™ 3.0 (Thermo® Finnigan®, San Jose, Calif.). Individual MS/MS spectra were extracted from the raw data file using the Sequest® Browser program CreateDta™ (owned by Thermo® Finnigan® San Jose, Calif.), with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The IonQuest™ and VuDta™ (owned by Thermo® Finnigan® San Jose, Calif.) programs were not used to further select MS/MS spectra for Sequest® analysis. MS/MS spectra were evaluated with the following TurboSequest™ parameters: peptide mass tolerance, 2.5; fragment ion tolerance, 0.0; maximum number of differential amino acids per modification, 4; mass type parent, average; mass type fragment, average; maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (as released on Aug. 24, 2004 and containing 27, 960 protein sequences). Cysteine carboxamidomethylation was specified as a static modification, and phosphorylation was allowed as a variable modification on serine, threonine, and tyrosine residues or on tyrosine residues alone. It was determined that restricting phosphorylation to tyrosine residues had little effect on the number of phosphorylation sites assigned. Furthermore, it should be noted that certain peptides were originally isolated in mouse and later normalized to human sequences as shown by Table 1/FIG. 2.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. The following Sequest scoring thresholds were used to select phosphopeptide assignments that are likely to be correct: RSp<6, XCorr≧2.2, and DeltaCN>0.099. Further, the assigned sequences could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria were satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### Example 2

### Production of Phospho-specific Polyclonal Antibodies for the Detection of Target Signal Protein/Polypepetide Phosphorylation

Polyclonal antibodies that specifically bind a target signal protein/polypepetide only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, as further described below. Production of exemplary polyclonal antibodies is provided below.

### A. Grb10 (Tyrosine 404)

A 10 amino acid phospho-peptide antigen, YGMLLy*QNYR (where y*=phosphotyrosine) that corresponds to the sequence encompassing the tyrosine 404 phosphorylation site in human Grb10 adaptor/scaffold protein (see Row 11 of Table 1; SEQ ID NO: 10), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific Grb10 (tyr404) polyclonal antibodies as described in Immunization/Screening below.

### B. ITSN2 (Tyrosine 261)

An 12 amino acid phospho-peptide antigen, SMSGy*LSGFQAR (where y*=phosphotyrosine) that corresponds to the sequence encompassing the tyrosine 261 phosphorylation site in human ITSN2 adaptor/scaffold protein (see Row 37 of Table 1 (SEQ ID NO: 36)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific ITSN2 (tyr 261) polyclonal antibodies as described in Immunization/Screening below.

### C KI-67 (Tyrosine 340)

A 13 amino acid phospho-peptide antigen, AVGASFPLy*EPAK (where y*=phosphotyrosine) that corresponds to the sequence encompassing the tyrosine 340 phosphorylation site in human KI-67 cell cycle regulation protein (see Row 50 of Table 1 (SEQ ID NO: 49), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific KI-67 (tyr340) antibodies as described in Immunization/Screening below.

### Immunization/Screening.

A synthetic phospho-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto a non-phosphorylated synthetic peptide antigen-resin Knotes column to pull out antibodies that bind the non-phosphorylated form of the phosphorylation site. The flow through fraction is collected and applied onto a phospho-synthetic peptide antigen-resin column to isolate antibodies that bind the phosphorylated form of the site. After washing the column extensively, the bound antibodies (i.e. antibodies that bind a phosphorylated peptide described in A-C above, but do not bind the non-phosphorylated form of the peptide) are eluted and kept in antibody storage buffer.

The isolated antibody is then tested for phospho-specificity using Western blot assay using an appropriate cell line that expresses (or overexpresses) target phospho-protein (i.e. phosphorylated Grb10, ITSN2.or KI-67), for example, K562, CTV-1 and KG1-A cells, respectively. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated phospho-specific antibody is used at dilution 1:1000. Phosphorylation-site specificity of the antibody will be shown by binding of only the phosphorylated form of the target protein. Isolated phospho-specific polyclonal antibody does not (substantially) recognize the target protein when not phosphorylated at the appropriate phosphorylation site in the non-stimulated cells (e.g. KI-67 is not bound when not phosphorylated at tyrosine 340).

In order to confirm the specificity of the isolated antibody, different cell lysates containing various phosphorylated signal transduction proteins other than the target protein are prepared. The Western blot assay is performed again using these cell lysates. The phospho-specific polyclonal antibody isolated as described above is used (1:1000 dilution) to test reactivity with the different phosphorylated non-target proteins on Western blot membrane. The phospho-specific antibody does not significantly cross-react with other phosphorylated signal transduction proteins, although occasionally slight binding with a highly homologous phosphorylation-site on another protein may be observed. In such case the antibody may be further purified using affinity chromatography, or the specific immunoreactivity cloned by rabbit hybridoma technology.

### Example 3

### Production of Phospho-specific Monoclonal Antibodies for the Detection of Target Signal Protein/Polypepetide Phosphorylation

Monoclonal antibodies that specifically bind a target signal protein/polypepetide only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, and harvesting spleen cells from such animals to produce fusion hybridomas, as further described below. Production of exemplary monoclonal antibodies is provided below.

### A. MAD2L1 (Tyrosine 199)

A 13 amino acid phospho-peptide antigen, VNSMVAy*KIPVND (where y*=phosphotyrosine) that corresponds to the sequence encompassing the tyrosine 199 phosphorylation site in human MAD2L1 cell cycle regulation protein (see Row 51 of Table 1 (SEQ ID NO: 50)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal MAD2L1 (tyr 199) antibodies as described in Immunization/Fusion/Screening below.

### B. HSC70 (Tyrosine 107)

An 10 amino acid phospho-peptide antigen, VQVEy*KGETK (where y*=phosphotyrosine) that corresponds to the sequence encompassing the tyrosine 107 phosphorylation site in human HSC70 chaperone (see Row 55 of Table 1 (SEQ ID NO: 54)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal HSC70 (tyr107) antibodies as described in Immunization/Fusion/Screening below.

### C. GCP3 (Tyrosine 256)

A 15 amino acid phospho-peptide antigen, DILy*FQGIDGK (where y*=phosphotyrosine) that corresponds to the sequence encompassing the tyrosine 256 phosphorylation site in human GCP3 cytoskeletal protein (see Row 80 of Table 1 (SEQ ID NO: 79), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal GCP3 (tyr256) antibodies as described in Immunization/Fusion/Screening below.

### Immunization/Fusion/Screening.

A synthetic phospho-peptide antigen as described in A-C above is coupled to KLH, and BAL,B/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the phospho-peptide and non-phospho-peptide forms of the antigen and by Western blot analysis (as described in Example 1 above). Colonies found to be positive by ELISA to the phospho-peptide while negative to the non-phospho-peptide are further characterized by Western blot analysis. Colonies found to be positive by Western blot analysis are subcloned by limited dilution. Mouse ascites are produced from a single clone obtained from subcloning, and tested for phospho-specificity (against the MAD2L 1, HSC70 or GCP3 phospho-peptide antigen, as the case may be) on ELISA. Clones identified as positive on Western blot analysis using cell culture supernatant as having phospho-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating phospho-specificity against the phosphorylated target (e.g. GCP3 phosphorylated at tyrosine 256).

### Example 4

### Production and Use of AQUA Peptides for the Quantification of Target Signal Protein/Polypepetide Phosphorylation

Heavy-isotope labeled peptides (AQUA peptides (internal standards)) for the detection and quantification of a target signal protein/polypepetide only when phosphorylated at the respective phosphorylation site disclosed herein (see Table 1/FIG. 2) are produced according to the standard AQUA methodology (see Gygi et al., Gerber et al., supra.) methods by first constructing a synthetic peptide standard corresponding to the phosphorylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

### A. GAPDH (Tyrosine 314)

An AQUA peptide comprising the sequence, LISWy*DNEFGYSNR (y*=phosphotyrosine; sequence incorporating ¹⁴C/¹⁵N-labeled leucine (indicated by bold L), which corresponds to the tyrosine 314 phosphorylation site in human GAPDH enzyme protein (see Row 99 in Table 1 (SEQ ID NO: 98)), is constructed according to standard synthesis techniques using, e.g., a Rainin/Protein Technologies, Inc., Symphony peptide synthesizer (see Merrifield, supra.) as further described below in Synthesis & MS/MS Signature. The GAPDH (tyr 314) AQUA peptide is then spiked into a biological sample to quantify the amount of phosphorylated GAPDH (tyr 314) in the sample, as further described below in Analysis & Quantification.

### B. H-Ras-1 (Tyrosine 157)

An AQUA peptide comprising the sequence QGVEDAFy*TLVR (y*=phosphotyrosine; sequence incorporating ¹⁴C.¹⁵N-labeled leucine (indicated by bold L), which corresponds to the tyrosine 157 phosphorylation site in human H-Ras-1 G protein or regulator protein (see Row 157 in Table 1 (SEQ ID NO: 156)), is constructed according to standard synthesis techniques using, e.g., a Rainin/Protein Technologies, Inc., Symphony peptide synthesizer (see Merrifield, supra.) as further described below in Synthesis & MS/MS Signature. The H-Ras-1 (tyr157) AQUA peptide is then spiked into a biological sample to quantify the amount of phosphorylated H-Ras-1 (tyr157) in the sample, as further described below in Analysis & Quantification.

### C G-alpha-s (Tyrosine 311)

An AQUA peptide comprising the sequence SKIEDy*FPEFAR (y*=phosphotyrosine; sequence incorporating ²⁴C/¹⁵N-labeled phenylalanine (indicated by bold F), which corresponds to the tyrosine 311 phosphorylation site in human G-alpha-s G protein or regulator protein (see Row 144 in Table 1 (SEQ ID NO: 143)), is constructed according to standard synthesis techniques using, e.g., a Rainin/Protein Technologies, Inc., Symphony peptide synthesizer (see Merrifield, supra.) as further described below in Synthesis & MS/MS Signature. The G-alpha-s (tyr311) AQUA peptide is then spiked into a biological sample to quantify the amount of phosphorylated G-alpha-s (tyr311) in the sample, as further described below in Analysis & Quantification.

### D. IL2RG (Tyrosine 325)

An AQUA peptide comprising the sequence, GLAESLQPDy*SER (y*=phosphotyrosine; sequence incorporating ¹⁴C/¹⁵N-labeled proline (indicated by bold P), which corresponds to the tyrosine 325 phosphorylation site in human IL2RG receptor/channel/transporter/cell surface protein (see Row 248 in Table 1 (SEQ ID NO: 247)), is constructed according to standard synthesis techniques using, e.g., a Rainin/Protein Technologies, Inc., Symphony peptide synthesizer (see Merrifield, supra.) as further described below in Synthesis & MS/MS Signature. The IL2RG (tyr325) AQUA peptide is then spiked into a biological sample to quantify the amount of phosphorylated IL2RG (tyr325) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino) methylene]-hexafluorophosphate

(1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a phosphorylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LTQ ion trap or TSQ Quantum triple quadrupole). On the LTQ, parent ions are isolated at 1.6 m/z width, the ion injection time being limited to 110 ms per microscan, with one microscans per peptide, and with an AGC setting of 1×10⁵; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. An isolated phosphorylation site-specific antibody that specifically binds a human signaling protein selected from Column A of Table 1, Rows 55, 106, 228, 157 and 240 only when phosphorylated at the tyrosine listed in corresponding Column D of Table 1, comprised within the phosphorylatable peptide sequence listed in corresponding Column E of Table 1 (SEQ ID NOs: 54, 105, 227, 156 and 239), wherein said antibody does not bind said signaling protein when not phosphorylated at said tyrosine.

47. An isolated phosphorylation site-specific antibody that specifically binds a human signaling protein selected from Column A of Table 1, Rows 55, 106, 228, 157 and 240 only when not phosphorylated at the tyrosine listed in corresponding Column D of Table 1, comprised within the phosphorylatable peptide sequence listed in corresponding Column E of Table 1 (SEQ ID NOs: 54, 105, 227, 156 and 239), wherein said antibody does not bind said signaling protein when phosphorylated at said tyrosine.

48. A method selected from the group consisting of:
(a) a method for detecting a human signaling protein selected from Column A of Table 1, Rows 55, 106, 228, 157 and 240 wherein said human signaling protein is phosphorylated at the tyrosine listed in corresponding Column D of Table 1, comprised within the phosphorylatable peptide sequence listed in corresponding Column E of Table 1 (SEQ ID NOs: 54, 105, 227, 156 and 239), comprising the step of adding an isolated phosphorylation-specific antibody according to claim 46, to a sample comprising said human signaling protein under conditions that permit the binding of said antibody to said human signaling protein, and detecting bound antibody;
(b) a method for quantifying the amount of a human signaling protein listed in Column A of Table 1, Rows 55, 106, 228, 157 and 240 that is phosphorylated at the corresponding tyrosine listed in Column D of Table 1, comprised within the phosphorylatable peptide sequence listed in corresponding Column E of Table 1 (SEQ ID NOs: 54, 105, 227, 156 and 239), in a sample using a heavy-isotope labeled peptide (AQUA TM peptide), said labeled peptide comprising a phosphorylated tyrosine at said corresponding lysine listed Column D of Table 1, comprised within the phosphorylatable peptide sequence listed in corresponding Column E of Table 1 as an internal standard; and
(c) a method comprising step (a) followed by step (b).

49. The method of claim 48, wherein said isolated phosphorylation-specific antibody is capable of specifically binding HSC70 only when phosphorylated at Y107, comprised within the phosphorylatable peptide sequence listed in Column E, Row 55, of Table 1 (SEQ ID NO: 54), wherein said antibody does not bind said protein when not phosphorylated at said tyrosine.

50. The method of claim 48, wherein said isolated phosphorylation-specific antibody is capable of specifically binding HSC70 only when not phosphorylated at Y107, comprised within the phosphorylatable peptide sequence listed in Column E, Row 55, of Table 1 (SEQ ID NO: 54), wherein said antibody does not bind said protein when phosphorylated at said tyrosine.

51. The method of claim 48, wherein said isolated phosphorylation-specific antibody is capable of specifically binding GLUD1 only when phosphorylated at Y451, comprised within the phosphorylatable peptide sequence listed in Column E, Row 106, of Table 1 (SEQ ID NO: 105), wherein said antibody does not bind said protein when not phosphorylated at said tyrosine.

52. The method of claim 48, wherein said isolated phosphorylation-specific antibody is capable of specifically binding GLUD1 only when not phosphorylated at Y451, comprised within the phosphorylatable peptide sequence listed in Column E, Row 106, of Table 1 (SEQ ID NO: 105), wherein said antibody does not bind said protein when phosphorylated at said tyrosine.

53. The method of claim 48, wherein said isolated phosphorylation-specific antibody is capable of specifically binding Lyn only when phosphorylated at Y306, comprised within the phosphorylatable peptide sequence listed in Column E, Row 228, of Table 1 (SEQ ID NO: 227), wherein said antibody does not bind said protein when not phosphorylated at said tyrosine.

54. The method of claim 48, wherein said isolated phosphorylation-specific antibody is capable of specifically binding Lyn only when not phosphorylated at Y306, comprised within the phosphorylatable peptide sequence listed in Column E, Row 228, of Table 1 (SEQ ID NO: 227), wherein said antibody does not bind said protein when phosphorylated at said tyrosine.

55. The method of claim 48, wherein said isolated phosphorylation-specific antibody is capable of specifically binding H-Ras-1 only when phosphorylated at Y157, comprised within the phosphorylatable peptide sequence listed in Column E, Row 157, of Table 1 (SEQ ID NO: 156), wherein said antibody does not bind said protein when not phosphorylated at said tyrosine.

56. The method of claim 48, wherein said isolated phosphorylation-specific antibody is capable of specifically binding H-Ras-1 only when not phosphorylated at Y157, comprised within the phosphorylatable peptide sequence listed in Column E, Row 157, of Table 1 (SEQ ID NO: 156), wherein said antibody does not bind said protein when phosphorylated at said tyrosine.

57. The method of claim 48, wherein said isolated phosphorylation-specific antibody is capable of specifically binding Hbb-b1 only when phosphorylated at Y36, comprised within the phosphorylatable peptide sequence listed in Column E, Row 240, of Table 1 (SEQ ID NO: 239), wherein said antibody does not bind said protein when not phosphorylated at said tyrosine.

58. The method of claim 48, wherein said isolated phosphorylation-specific antibody is capable of specifically binding Hbb-b1 only when not phosphorylated at Y36, comprised within the phosphorylatable peptide sequence listed in Column E, Row 240, of Table 1 (SEQ ID NO: 239), wherein said antibody does not bind said protein when phosphorylated at said tyrosine.
